(19) United States
(12) Patent Application Publication
(10) Pub. No.: US 2025/0109173 A1
(43) Pub. Date: Apr. 3, 2025

(54) RECOMBINANT POLYPEPTIDES CONTAINING AT LEAST ONE IMMUNOGENIC FRAGMENT AND ANTIBODY FC REGION AND USES THEREOF

(71) Applicant: BOOST BIOPHARMA, INC., Amherst, MA (US)

(72) Inventors: Fritz Schomburg, Madison, WI (US); David Rancour, Madison, WI (US)

(73) Assignee: BOOST BIOPHARMA, INC., Amherst, MA (US)

(21) Appl. No.: 18/725,518

(22) PCT Filed: Dec. 30, 2022
(86) PCT No.: PCT/US2022/082653

§ 371 Date: Jun. 28, 2024

Related U.S. Application Data
(60) Provisional application No. 63/295,776, filed on Dec. 31, 2021.

(51) Int. Cl. C07K 14/005; A61K 39/00

(52) U.S. Cl. CPC C07K 14/005; A61K 39/00; A61K 2039/55505; C07K 2319/30; C12N 2770/20022; C12N 2770/20034

(57) ABSTRACT

Provided is a recombinant polypeptide containing at least one immunogenic fragment of Severe Acute Respiratory Syndrome Coronavirus 2 (SARS-COV-2) spike glycoprotein, and pharmaceutical compositions containing the same.

### CROSS-REFERENCE TO PRIOR APPLICATION

This application claims benefit to U.S. Provisional Patent Application No. 63/295,776, filed Dec. 31, 2021, which is hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,404,167 Byte XML file named “SEQL_766399,” created on Dec. 13, 2022.

### BACKGROUND OF THE INVENTION

The rapid evolution of new SARS-COV-2 variants containing mutations that alter the amino acid sequence of the Spike protein resulting in altered function, and altered resistance to native immune defenses and to immune defenses elicited by currently marketed vaccines has led to a need for alternative platforms that allow for incorporation of new variant mutations in a robust vaccine.

### BRIEF SUMMARY OF THE INVENTION

The present invention relates to recombinant polypeptides that include at least one immunogenic fragment of Severe Acute Respiratory Syndrome Coronavirus 2 (SARS-COV-2) spike glycoprotein and an antibody Fc region. In some embodiments, the recombinant polypeptide includes more than one immunogenic fragment, e.g., two, three, four, five, or more immunogenic fragments. In some embodiments, the recombinant polypeptide includes one or more immunogenic fragments of Severe Acute Respiratory Syndrome Coronavirus (SARS-COV) and/or Middle Eastern Respiratory Syndrome Coronavirus (MERS-COV) and an antibody Fc region, optionally in combination with one or more SARS-COV-2 immunogenic fragments.

The present invention further relates to pharmaceutical compositions, such as vaccines, that include the recombinant polypeptide. In some embodiments, the pharmaceutical composition includes an adjuvant.

The present invention also relates to a method for preventing, inhibiting, reducing, eliminating, protecting, and/or delaying the onset of an infection or an infectious clinical condition caused by a beta coronavirus in a subject, wherein the method includes administering to the subject at least one recombinant polypeptide of the invention or a pharmaceutical composition including the same.

The present invention further relates to a method for inducing an immune response against a coronavirus in a subject, wherein the method includes administering to the subject at least one recombinant polypeptide of the invention or a pharmaceutical composition including the same.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph depicting Spike S1 protein IgG response in Rhesus macaques at certain time points (in weeks) after initial injection with an exemplary construct.

FIG. 2A is an image showing CHO cell expression of CTD_short_a-Fc (LS2330) after 5 days. Protein was affinity purified using Protein A agarose and analyzed by reducing SDS-PAGE and detected by Coomassie R-250 staining. M-Molecular weight markers. As can be seen in each of FIGS. 2A-2D, the constructs were resistant to proteolytic degradation during expression thus facilitating higher yields of intact, soluble protein

FIG. 2B is an image showing CHO cell expression of CTD_long_a-Fc (LS3472) after 5 days. Protein was affinity purified using Protein A agarose and analyzed by reducing SDS-PAGE and detected by Coomassie R-250 staining. Protein loads per lane are indicated.

FIG. 2C is an image showing CHO cell expression of CTD-deletion series after 5 days. Protein was affinity purified using Protein A agarose from equal volumes of transfected culture. Eluted protein was loaded based on volume, separated by reducing SDS-PAGE, and detected by Coomassie R-250 staining. Load volumes were twice as large for samples (left to right) CTD_vs_a-Fc to RBD_e-Fc compared to the samples on the left of the gel (CTD_long_b-Fc to CTD_short_h-Fc).

FIG. 2D is an image showing CHO cell expression of CTD_short_i-Fc (LS2371) after 5 days. Protein was affinity purified using Protein A agarose and analyzed by reducing SDS-PAGE and detected by Coomassie R-250 staining.

FIG. 3 is an image showing CHO cell expression of (CTD_short_d)₂-Fc (clone 1 through 4 correspond to strains LS2397 through 2400; SEQ ID NO: 161) and (CTD_short_i)₂-Fc (clone 1 through 4 correspond to strains LS2401 through 2404; SEQ ID NO: 163) after 4 or 7 days as indicated. Protein was affinity purified using Protein A agarose from equal volumes of transfected culture. Eluted protein was loaded based on volume, separated by reducing SDS-PAGE, and detected by Coomassie R-250 staining. M-Molecular weight markers.

FIG. 4 is an image showing CHO cell expression of mutant (CTD_short_i)₂-Fc constructs wherein amino acid mutations corresponding to newly identified SARS-COV-2 variants have been added to both CTD domains (D1 and D2) or second domain (D2) only of the CTD dimer. The mutants tested include (a) D1 and D2 mutations for hybrid P.1 and CAL.20C variants; K417T, L452R, E484K, N501 Y (Strain 2435), (b) D2 mutations for 501. V2 variant/B.1.351, K417N, E484K, N501Y (Strains 2421), and (c) D2 mutations for hybrid P.1 and CAL.20C variants; K417T, L452R, E484K, N501Y (Strain 2423). Protein was affinity purified using Protein A agarose from equal volumes of 4-day transfected culture. Eluted protein was loaded based on volume, separated by reducing SDS-PAGE, and detected by Coomassie R-250 staining. M-Molecular weight markers. Protein loads were 3.48 (strain 2435), 4.52 (strain 2421), and 3.14 μg (strain 2423).

FIGS. 5A, 5B, and 5C are graphs showing the results of neutralization assays using serum samples from animals P0101 and P0102 immunized with SEQ ID NO: 73 (LS2330 [CTD_short_a-Fc], which were the subject of analysis discussed in Example 1. Infectivity of serum neutralized pseudotypes virus using 295T/ACE2 target cells was quantified by measuring NanoLuc luciferase activity (RLU) and graphed on the y-axis. Reciprocal serum dilution is shown on the x-axis.

FIG. 6 is a set of SDS-PAGE gel photographs depicting expression of certain immunogenic fragment constructs in CHO-S cells or in ExpiCHO cells. “M1” and “M2” denote markers. Full-length protein is seen as the primary band at about 55-60 kDa. A dimer of the protein (an in vitro result of high concentration of purified protein) is found at about 120 kDa. Degraded protein can be seen at about 38 kDa with respect to CTD-short_g grown in CHO-S cells.

### DETAILED DESCRIPTION OF THE INVENTION

Recombinant polypeptides of the invention can include any suitable immunogenic fragment or fragments of the Severe Acute Respiratory Syndrome Coronavirus 2 (SARS-COV-2) spike glycoprotein and any suitable antibody Fc region. In some embodiments, an immunogenic fragment comprises, consist of, or consist essentially of, the N-terminal domain of the S1 subunit, the C-terminal domain of the S1 subunit, or both. In certain embodiments, an immunogenic fragment can include the complete SARS-COV-2 spike glycoprotein. Recombinant polypeptides of the invention include at least one immunogenic fragment, and can contain two, three, four, five, six, seven, eight, nine, ten, eleven, twelve, thirteen, fourteen, fifteen, or more such immunogenic fragments.

In some embodiments, one or more of the immunogenic fragments are identical to a wild-type SARS-COV-2 spike glycoprotein, or any portion thereof. Wild-type spike glycoproteins include those of any SARS-COV-2 strain that has been isolated from a subject. Examples include Wuhan-Hu-1, VOC 202012/01/B.1.1.7 (Alpha or UK), VOC-202102/02 (B.1.1.7 with E484K) (UK), 501.V2/B.1.351 (South Africa), B.1.429/CAL.20C (California), B.1.525, and Lineage P.1 (Gamma or Brazil), B.1.427 (Epsilon), B.1.429 (Epsilon), B.1.617.1, B.1.617.2 (Delta), B.1.526 (Iota), B.1.617.3, B.1, A.2.5, C.36.3, B.1.1.318, B.1.351, B.1.621, B.1.525, P.1.1, P.2 (Zeta), B.1.623, R.1, B.1.1.7, B.1.351, B.1.351.3, A.30 (EPI_ISL_1347942), B.1.1.529 (Omicron), BA.2.75, BA.2.75.2, BA.4, BA.4.6, BA.5, BA.5.2.6, BF.7, BF.11, BN.1, and BQ.1. In some embodiments, one or more of the immunogenic fragments are identical to a wild-type SARS-COV or MERS spike glycoprotein. Wild-type SARS-COV and MERS spike glycoproteins include those of any SARS-COV or MERS strain that has been isolated from a subject. Examples of such strains include SARS coronavirus Tor2 (GenBank accession number NC_004718.3) and MERS coronavirus (GenBank accession number NC_019843.3).

In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to a wild-type SARS-COV-2 spike glycoprotein, or any portion thereof. In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to a wild-type SARS-COV or MERS spike glycoprotein, or any portion thereof.

In some embodiments, one or more of the immunogenic fragments are identical to a wild-type SARS-COV-2 spike glycoprotein, or any portion thereof, except for at one or more of the following positions of the amino acid sequence: L5, A67, H69, V70, D80, T95, G142, Y144, E154, F157, D253, R346, G339, S371, S373, S375, K417, N440, G446, Y449, L452, F456, S477, T478, E484, F486, N487, Q493, Q496, Q498, T500, N501, G502, Y505, D614, Q677, P681, A701, T791, T859, F888, D950, and Q1071, wherein the positions of the listed amino acid residues correspond to the wild-type amino acid sequence QHD43416 (ncbi.nlm.nih.gov/protein/QHD43416). The mutations at these positions can be any suitable mutation, including conservative and non-conservative amino acid mutations. For instance, a conservative substitution can replace one aliphatic amino acid (i.e., Glycine, Alanine, Valine, Leucine, Methionine or Isoleucine) for another, one polar, uncharged R group amino acid (i.e., Serine, Cysteine, Threonine, Proline, Asparagine, or Methionine) for another, one positively charged R group amino acid (i.e., Histidine, Lysine, or Arginine) for another, one negatively charged R group amino acid (i.e., Aspartate or Glutamate) for another, and one non-polar, aromatic R group amino acid (i.e., Phenylalanine, Tyrosine, or Tryptophan) for another. In some embodiments, one or more of the immunogenic fragments are identical to a wild-type SARS-CoV-2 spike glycoprotein, or any portion thereof, except for one or more of the following amino acid substitutions and deletions: L5F, A67V, 69del, 70del, D80G, T95I, G142D, 144del, E154K, F157S, D253G, R346K, G339D, S371L, S373P, S375F, N440K, G446S, L452R, S477N, E484K, E484Q, E484A, K417N, K417T, T478K, Q493R, Q496S, Q498R, N501Y, Y505H, D614G, Q677H, T791I, P681H, P681R, A701V, F888L, T859N, D950H, D950N, and Q1071H, wherein the listed amino acid substitutions and deletions are relative to the wild-type amino acid sequence QHD43416 (ncbi.nlm.nih.gov/protein/QHD43416). In one embodiment, the one or more amino acid substitutions and/or deletions is L452R. In another embodiment, the one or more amino acid substitutions and/or deletions is E484K. In a further embodiment, the one or more protein substitutions and/or deletions are K417N, E484K, and N501Y. In yet another embodiment, the one or more substitutions are and/or deletions are K417T, E484K, and N501Y. In another embodiment, the one or more amino acid substitutions and/or deletions are N501Y, 69del, 70del, and P681H. In another embodiment, the one or more amino acid substitutions and/or deletions are K417T, L452R, E484K, and N501Y. In another embodiment, the one or more amino acid substitutions and/or deletions are L452R and T478K. In another embodiment, the one or more amino acid substitutions and/or deletions are K417N, L452R, and T478K. In another embodiment, the one or more amino acid substitutions and/or deletions are R346K, E484K, and N501Y. In another embodiment, the one or more amino acid substitutions and/or deletions are R346K, T478R, and E484K. In another embodiment, the one or more amino acid substitutions and/or deletions are R346K, K417N, L452R, 478K, E484K, and N501Y. In another embodiment, the one or more amino acid substitutions and/or deletions are R346K, K417N, L452R, T478R, E484K, and N501Y. In another embodiment, the one or more amino acid substitutions and/or deletions are G339D, S371L, S373P, S375F, K417N, N440K, G446S, S477N, T478K, E484A, Q493K, G496S, Q498R, N501Y, and Y505H. In another embodiment, the one or more amino acid substitutions and/or deletions are G339D, S371L, S373P, S375F, K417N, N440K, G446S, S477N, T478K, E484A, Q493R, G496S, Q498R, N501Y, and Y505H. In another embodiment, the one or more amino acid substitutions and/or deletions are G339D, R346K, S371L, S373P, S375F, K417N, N440K, G446S, L452R, S477N, T478K, E484K, Q493R, G496S, Q498R, N501Y, and Y505H. In another embodiment, the one or more amino acid substitutions and/or deletions are G339D, R346K, S371L, S373P, S375F, K417N, N440K, G446S, L452R, S477N, T478K, E484A, Q493R, G496S, Q498R, N501Y, and Y505H. In another embodiment, the one or more amino acid substitutions and/or deletions are G339D, R346K, S371L, S373P, S375F, K417N, N440K, G446S, L452R, S477N, T478R, E484A, Q493R, G496S, Q498R, N501Y, and Y505H. In another embodiment, the one or more amino acid substitutions and/or deletions are G339D, R346K, S371L, S373P, S375F, K417N, N440K, G446S, L452R, S477N, T478R, E484K, Q493R, G496S, Q498R, N501Y, and Y505H.

The subject can be mammalian, including human, non-human primate, horse, pig, cattle, cat, dog, sheep, mink, rodent, hamster, or bat. Subjects can further include western lowland gorilla, northern white-cheeked gibbon, Sumatran orangutan, crab-eating macaque, drill, proboscis monkey, bonobo, chimpanzee, ugandan red colobus, red-shanked douc, golden snub-nosed monkey, green monkey, patas monkey, rhesus macaque, olive baboon, gelade, sooty mangabey, southern pig-tailed macaque, angola colobus, coquerel's sifaka, Gambian pouched rat, Chinese hamster, common gund, beluga whale, blue-eyed black lemur, indri, narwhal, narrow-ridged finless porpoise, harbour porpoise, minke whale, Antarctic minke whale, gray whale, spalax, white-tailed deer, reindeer, southern tamandua, Stephens's kangaroo rat, Père David's deer, transcaucasian mole vole, long-finned pilot whale, Pacific white-sided dolphin, baiji, giant anteater, muskrat, killer whale, common bottlenose dolphin, aye-aye, fat-tailed dwarf lemur, thirteen-lined ground squirrel, yellow-bellied marmot, alpine marmot, golden hamster, sperm whale, daurian ground squirrel, gobi jerboa, barbry sheep, pronghorn, Nancy ma's night monkey, hirola, American bison, zebu, wild yak, cattle, water buffalo, white-eared titi, common marmoset, wild goat, goat, Panamanian white-faced capuchin, cat, Masai giraffe, Nilgiri tahr, Candian lynx, coquerel's giant mouse lemur, Siberian musk deer, sunda clouded leopard, clouded leopard, okapi, sheep, jaguar, leopard, Siberian tiger, Tibetan antelope, little pocket mouse, deer mouse, white-faced saki, cougar, black-capped squirrel monkey, tufted capuchin, arctic ground squirrel, bos indicus x bos Taurus, cheetah, mantled howler, Geoffroy's spider monkey, Damaraland mole-rat, naked mole-rat, hippopotamus, snowshoe hare, Dama gazelle, European rabbit, scimitar oryx, emperor tamarin, and alpaca.

In some embodiments, one or more of the immunogenic fragments contain one or more mutations, such that the one or more immunogenic fragments are not identical to a wild-type SARS-COV-2 spike glycoprotein, or any portion thereof. The one or more mutations can be any suitable mutation and/or deletion. For example, an immunogenic fragment can contain sequences from two, three, four, five, six, seven, eight, nine, ten, or more strains, such that the resulting fragment is no longer identical to any of its parent strains. In this way, a single immunogenic fragment can present epitopes from multiple wild-type SARS-COV-2 spike glycoproteins, or any portion thereof. This can lead to a more robust immune response and increased immune protection from a range of SARS-COV-2 strains in a subject when a recombinant polypeptide of the invention containing one or more such immunogenic fragments, or a pharmaceutical composition containing the same, is administered to the subject.

In some embodiments, the nucleic acid sequence encoding an immunogenic fragment includes one, two, three, four, five, six, seven, eight, nine, ten, twenty, thirty, forty, or more point mutations and/or deletions in comparison to the nucleic acid sequence encoding the corresponding fragment of a wild-type or mutant glycoprotein. In some embodiments, the amino acid sequence of an immunogenic fragment includes one, two, three, four, five, six, seven, eight, nine, ten, twenty, thirty, forty, or more substitutions and/or deletions in comparison to the amino acid sequence encoding corresponding fragment of a wild-type or mutant glycoprotein.

In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 1 (CTD_long_a), SEQ ID NO: 3 (CTD_long_a_D614G), SEQ ID NO: 5 (CTD_long_a-Dimer), SEQ ID NO: 7 (CTD_long_b), SEQ ID NO: 9 (CTD_long_c), SEQ ID NO: 11 (CTD_long_d), SEQ ID NO: 13 (CTD_long_e), SEQ ID NO: 15 (CTD_long_f), SEQ ID NO: 17 (CTD_long_g), SEQ ID NO: 19 (CTD_long_h), SEQ ID NO: 21 (CTD_long_i), SEQ ID NO: 23 (CTD_short_a), SEQ ID NO: 25 (CTD_short_b), SEQ ID NO: 27 (CTD_short_c), SEQ ID NO: 29 (CTD_short_d), SEQ ID NO: 31 (CTD_short_e), SEQ ID NO: 33 (CTD_short_f), SEQ ID NO: 35 (CTD_short_g), SEQ ID NO: 37 (CTD_short_h), SEQ ID NO: 39 (CTD_short_i), SEQ ID NO: 41 (CTD_vs_a), SEQ ID NO: 43 (CTD_vs_b), SEQ ID NO: 45 (CTD_vs_c), SEQ ID NO: 47 (CTD_vs_d), SEQ ID NO: 49 (CTD_vs_e), SEQ ID NO: 51 (RBD_a), SEQ ID NO: 53 (RBD_b), SEQ ID NO: 55 (RBD_c), SEQ ID NO: 57 (RBD_d), SEQ ID NO: 59 (RBD_e), SEQ ID NO: 61 (NTD_long_a), SEQ ID NO: 63 (NTD_short_a), SEQ ID NO: 171 (RBD-tight), SEQ ID NO: 173 ((RBD-tight)₂), SEQ ID NO: 175 (RBD-extended), SEQ ID NO: 177 ((RBD-extended)₂), SEQ ID NO: 179 (RBD), SEQ ID NO: 181 ((RBD)₂), SEQ ID NO: 183 ((CTD_short_d)₂), SEQ ID NO: 185 ((CTD_short_i)₂), SEQ ID NO: 187 ((CTD_short_i)₂-mod. 1), SEQ ID NO: 189 ((CTD_short_i)₂-mod. 2), SEQ ID NO: 191 ((CTD_short_i)₂-mod. 3), SEQ ID NO: 199 (SARS-2003, SARS_short_h), SEQ ID NO: 201 (SARS-2003, SARS_short_i), SEQ ID NO: 203 (MERS_Lytic_a), SEQ ID NO: 205 (MERS_Lytic_b), SEQ ID NO: 207 (MERS_Lytic_c), SEQ ID NO: 209 (MERS_Lytic_d), SEQ ID NO: 211 (MERS_Lytic_e), SEQ ID NO: 213 (MERS_Lytic_f), SEQ ID NO: 215 (MERS_Lytic_g), and SEQ ID NOs: 705-716. In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 185 ((CTD_short_i)₂), SEQ ID NO: 187 ((CTD_short_i)₂-mod. 1), SEQ ID NO: 189 ((CTD_short_i)₂-mod. 2), and SEQ ID NO: 191 ((CTD_short_i)₂-mod. 3). In further embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to the amino acid sequence of SEQ ID NO: 185 ((CTD_short_i)₂). In further embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 183 ((CTD_short_d)₂), SEQ ID NO: 39 (CTD_short_i), SEQ ID NO: 29 (CTD_short_d), SEQ ID NO: 31 (CTD_short_e), SEQ ID NO: 37 (CTD_short_h), and SEQ ID NO: 23 (CTD_short_a). In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to the amino acid sequence represented by SEQ ID NO: 39. In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to the amino acid sequence represented by SEQ ID NO: 185. In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to the amino acid sequence represented by SEQ ID NO: 190. In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NOs: 705-716.

In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 245 (CTD_short_h) 2), SEQ ID NO: 249 (LS2435 Single domain_(GAMMA-Plus)), SEQ ID NO: 253 (BETA_CTD_short_i), SEQ ID NO: 257 (GAMMA_CTD_short_i), SEQ ID NO: 261 (GAMMA_CTD_short_i), SEQ ID NO: 265 (DELTA-Plus_CTD_short_i), SEQ ID NO: 269 (MU_CTD_short_i), SEQ ID NO: 273 (A.30_CTD_short_i), SEQ ID NO: 277 (DELTA-Plus+MU_CTD_short_i), SEQ ID NO: 281 (DELTA-Plus+A.30_CTD_short_i), SEQ ID NO: 285 (BETA_CTD_short_i)₂), SEQ ID NO: 289 ((GAMMA_CTD_short_i)₂), SEQ ID NO: 293 ((DELTA_CTD_short_i)₂), SEQ ID NO: 297 ((DELTA-Plus_CTD_short_i)₂), SEQ ID NO: 301 ((MU_CTD_short_i)₂), SEQ ID NO: 305 ((A.30_CTD_short_i)₂), SEQ ID NO: 309 ((DELTA-Plus+MU_CTD_short_i)₂), SEQ ID NO: 313 ((DELTA-Plus+A.30_CTD_short_i)₂), SEQ ID NO: 317 (Omicron_Boost_CTD_short_i), SEQ ID NO: 321 ((Omicron_Boost_CTD_short_i)₂), SEQ ID NO: 325 (Omicron_CTD_short_i), SEQ ID NO: 329 ((Omicron_CTD_short_i)₂), SEQ ID NO: 333 (OΔ+μ_1_CTD_short_i), SEQ ID NO: 337 ((Δ+μ_1_CTD_short_i)₂), SEQ ID NO: 341 (Δ+μ_2_CTD_short_i), SEQ ID NO: 345 ((OΔ+μ_2_CTD_short_i)₂), SEQ ID NO: 349 (OΔ+μ_3_CTD_short_i), SEQ ID NO: 353 ((Δ+μ_3_CTD_short_i)₂), SEQ ID NO: 357 (Δ+μ_3_CTD_short_i), SEQ ID NO: 361 ((OΔ+μ_4_CTD_short_i)₂), SEQ ID NO: 365 (CTD-gCd01), SEQ ID NO: 366 (CTD-gCd02), SEQ ID NO: 367 (CTD-gCd03), SEQ ID NO: 368 (CTD-gCd04), SEQ ID NO: 369 (CTD-gCd05), SEQ ID NO: 370 (CTD-gCd06), SEQ ID NO: 371 (CTD-gCd07), SEQ ID NO: 372 (CTD-gCd08), SEQ ID NO: 373 (CTD-gCd09), SEQ ID NO: 374 (CTD-gCd10), SEQ ID NO: 375 (CTD-gCd11), SEQ ID NO: 376 (CTD-gCd12), SEQ ID NO: 377 (CTD-gCd13), SEQ ID NO: 378 (CTD-gCd14), SEQ ID NO: 379 (CTD-gCd15), SEQ ID NO: 380 (CTD-gCd16), SEQ ID NO: 381 (CTD-gCd17), SEQ ID NO: 382 (CTD-gCd18), SEQ ID NO: 383 (CTD-gCd19), SEQ ID NO: 384 (CTD-gCd20), SEQ ID NO: 385 (CTD-gCd21), SEQ ID NO: 386 (CTD-gCd22), SEQ ID NO: 387 (CTD-gCd23), SEQ ID NO: 388 (CTD-gCd24), SEQ ID NO: 389 (CTD-gCd25), SEQ ID NO: 390 (CTD-gCd26), SEQ ID NO: 391 (CTD-gCd27), SEQ ID NO: 392 (CTD-gCd28), SEQ ID NO: 393 (CTD-gCd29), SEQ ID NO: 394 (CTD-gCd30), SEQ ID NO: 395 (CTD-gCd31), SEQ ID NO: 396 (CTD-gCd32), SEQ ID NO: 397 (CTD-gCd33), SEQ ID NO: 398 (CTD-gCd34), SEQ ID NO: 399 (CTD-gCd35), SEQ ID NO: 400 (CTD-gCd36), SEQ ID NO: 401 (CTD-gCd37), SEQ ID NO: 402 (CTD-gCd38), SEQ ID NO: 403 (CTD-gN01), SEQ ID NO: 404 (CTD-gN02), SEQ ID NO: 405 (CTD-gN03), SEQ ID NO: 406 (CTD-gN04), SEQ ID NO: 407 (CTD-gN05), SEQ ID NO: 408 (CTD-gN06), SEQ ID NO: 409 (CTD-gN07), SEQ ID NO: 410 (CTD-gN08), SEQ ID NO: 411 (CTD-gN09), SEQ ID NO: 412 (CTD-gN10), SEQ ID NO: 413 (CTD-gN11), SEQ ID NO: 414 (CTD-gN12), SEQ ID NO: 415 (CTD-gN13) SEQ ID NO: 416 (CTD-gN14), SEQ ID NO: 417 (CTD-gN15), SEQ ID NO: 418 (CTD-gN16), SEQ ID NO: 419 (CTD-gN17), SEQ ID NO: 420 (CTD-gN18), SEQ ID NO: 421 (CTD-gN19), SEQ ID NO: 422 (CTD-gN20), SEQ ID NO: 423 (CTD-gN21), SEQ ID NO: 424 (CTD-gN22), SEQ ID NO: 425 (CTD-gN23), SEQ ID NO: 426 (CTD-gN24), SEQ ID NO: 427 (CTD-gN25), SEQ ID NO: 428 (CTD-gN26), SEQ ID NO: 429 (CTD-gN27), SEQ ID NO: 430 (CTD-gN28), SEQ ID NO: 431 (CTD-gN29), SEQ ID NO: 432 (CTD-gN30), SEQ ID NO: 433 (CTD-gN31), SEQ ID NO: 434 (CTD-gN32), SEQ ID NO: 435 (CTD-gN33), SEQ ID NO: 436 (CTD-gN34), SEQ ID NO: 437 (CTD-gN35), SEQ ID NO: 438 (CTD-gN36), SEQ ID NO: 439 (CTD-gN37), SEQ ID NO: 440 (CTD-gN38), SEQ ID NO: 441 (CTD-gN39), SEQ ID NO: 442 (CTD-gN40), SEQ ID NO: 443 (CTD-gN41), SEQ ID NO: 444 (CTD-gN42), SEQ ID NO: 445 (CTD-gN43), SEQ ID NO: 446 (CTD-gN44), SEQ ID NO: 447 (CTD-gN45), SEQ ID NO: 448 (CTD-gN46), SEQ ID NO: 449 (CTD-gN47), and SEQ ID NOs: 705-716.

In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 277 (DELTA-Plus+MU_CTD_short_i), SEQ ID NO: 281 (DELTA-Plus+A.30_CTD_short_i), SEQ ID NO: 309 ((DELTA-Plus+MU_CTD_short_i)₂), SEQ ID NO: 313 ((DELTA-Plus+A.30_CTD_short_i)₂), SEQ ID NO: 333 (Δ+μ_1_CTD_short_i), SEQ ID NO: 337 ((Δ+μ_1_CTD_short_i)₂, and SEQ ID NOs: 705-716. In some embodiments, an immunogenic fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NOs: 705-716.

In some embodiments, the recombinant polypeptide comprises at least one immunogenic fragment that comprises, consists of, or consists essentially of an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 171, 173, 175, 177, 179, 181, 183, 185, 187, 189, 191, 199, 201, 203, 205, 207, 209, 211, 213, 215, and 705-716. In some embodiments, an immunogenic fragment comprises, consists of, or consists essentially of an amino acid sequence selected from the group consisting of SEQ ID NO: 185 ((CTD_short_i)₂), SEQ ID NO: 187 ((CTD_short_i)₂-mod. 1), SEQ ID NO: 189 ((CTD_short_i)₂-mod. 2), and SEQ ID NO: 191 ((CTD_short_i)₂-mod. 3). In an embodiment, an immunogenic fragment comprises, consists of, or consists essentially of the amino acid sequence represented by SEQ ID NO: 185. In an embodiment, an immunogenic fragment comprises, consists of, or consists essentially of the amino acid sequence represented by SEQ ID NO: 191. In further embodiments an immunogenic fragment comprises, consists of, or consists essentially of the amino acid sequence of SEQ ID NO: 185 ((CTD_short_i)₂). In further embodiments, an immunogenic fragment comprises, consists of, or consists essentially of an amino acid sequence selected from the group consisting of SEQ ID NO: 183 ((CTD_short_d)₂), SEQ ID NO: 39 (CTD_short_i), SEQ ID NO: 29 (CTD_short_d), SEQ ID NO: 31 (CTD_short_e), SEQ ID NO: 37 (CTD_short_h), and SEQ ID NO: 23 (CTD_short_a). In further embodiments, an immunogenic fragment comprises, consists of, or consists essentially of an amino acid sequence selected from the group consisting of SEQ ID NOs: 705-716.

In some embodiments, the recombinant polypeptide comprises, consists of, or consists essentially of at least one immunogenic fragment that comprises, consists of, or consists essentially of an amino acid sequence selected from the group consisting of SEQ ID NOS: 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, and 365-449. In some embodiments, the recombinant polypeptide comprises, consists of, or consists essentially of at least one immunogenic fragment that comprises, consists of, or consists essentially of an amino acid sequence selected from the group consisting of SEQ ID NOs: 277, 281, 309, 313, 333, and 337.

In some embodiments, the recombinant polypeptide comprises at least one immunogenic fragment that is encoded by a nucleotide sequence that comprises, consists of, or consists essentially of a nucleotide sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 172, 174, 176, 178, 180, 182, 184, 186, 188, 190, 192, 200, 202, 204, 206, 208, 210, 212, 214, 216, and 717-728. In some embodiments, the recombinant polypeptide comprises, consists of, or consists essentially of at least one immunogenic fragment that is encoded by a nucleotide sequence that comprises, consists of, or consists essentially of a nucleotide sequence selected from the group consisting of SEQ ID NOs: 246, 250, 254, 258, 262, 266, 270, 274, 278, 282, 286, 290, 294, 298, 302, 306, 310, 314, 318, 322, 326, 330, 334, 338, 342, 346, 350, 354, 358, 362, and 450-534. In some embodiments, the recombinant polypeptide comprises at least one immunogenic fragment that is encoded by a nucleotide sequence that comprises, consists of, or consists essentially of a nucleotide sequence selected from the group consisting of SEQ ID NOs: 278, 282, 310, 314, 334, and 338. In some embodiments, the recombinant polypeptide comprises at least one immunogenic fragment that is encoded by a nucleotide sequence that comprises, consists of, or consists essentially of a nucleotide sequence selected from the group consisting of SEQ ID NOs: 717-728.

In some embodiments, the recombinant polypeptide includes at least two immunogenic fragments. For example, the recombinant polypeptide can include two, three, four, five, six, seven, eight, nine, ten, or more immunogenic fragments. In some embodiments, the recombinant polypeptide includes at least three immunogenic fragments. In some embodiments, the recombinant polypeptide includes at least three immunogenic fragments, of which at least one is a SARS-COV-2 spike glycoprotein fragment as described herein, at least one is a SARS-CoV spike glycoprotein fragment as described herein, and at least one is a MERS-COV spike glycoprotein fragment as described herein. In some embodiments, the recombinant polypeptide includes at least three immunogenic fragments, of which at least one is a SARS-COV-2 spike glycoprotein fragment, wherein the SARS-COV-2 fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 1 (CTD_long_a), SEQ ID NO: 3 (CTD_long_a_D614G), SEQ ID NO: 5 (CTD_long_a-Dimer), SEQ ID NO: 7 (CTD_long_b), SEQ ID NO: 9 (CTD_long_c), SEQ ID NO: 11 (CTD_long_d), SEQ ID NO: 13 (CTD_long_e), SEQ ID NO: 15 (CTD_long_f), SEQ ID NO: 17 (CTD_long_g), SEQ ID NO: 19 (CTD_long_h), SEQ ID NO: 21 (CTD_long_i), SEQ ID NO: 23 (CTD_short_a), SEQ ID NO: 25 (CTD_short_b), SEQ ID NO: 27 (CTD_short_c), SEQ ID NO: 29 (CTD_short_d), SEQ ID NO: 31 (CTD_short_e), SEQ ID NO: 33 (CTD_short_f), SEQ ID NO: 35 (CTD_short_g), SEQ ID NO: 37 (CTD_short_h), SEQ ID NO: 39 (CTD_short_i), SEQ ID NO: 41 (CTD_vs_a), SEQ ID NO: 43 (CTD_vs_b), SEQ ID NO: 45 (CTD_vs_c), SEQ ID NO: 47 (CTD_vs_d), SEQ ID NO: 49 (CTD_vs_e), SEQ ID NO: 51 (RBD_a), SEQ ID NO: 53 (RBD_b), SEQ ID NO: 55 (RBD_c), SEQ ID NO: 57 (RBD_d), SEQ ID NO: 59 (RBD_e), SEQ ID NO: 61 (NTD_long_a), SEQ ID NO: 63 (NTD_short_a), SEQ ID NO: 171 (RBD-tight), SEQ ID NO: 173 ((RBD-tight)₂), SEQ ID NO: 175 (RBD-extended), SEQ ID NO: 177 ((RBD-extended)₂), SEQ ID NO: 179 (RBD), SEQ ID NO: 181 ((RBD)₂), SEQ ID NO: 183 ((CTD_short_d)₂), SEQ ID NO: 185 ((CTD_short_i)₂), SEQ ID NO: 187 ((CTD_short_i)₂-mod. 1), SEQ ID NO: 189 ((CTD_short_i)₂-mod. 2), SEQ ID NO: 191 ((CTD_short_i)₂-mod. 3), and SEQ ID NOs: 705-716; at least one is a SARS-COV spike glycoprotein fragment, wherein the SARS-COV fragment comprises, consists of, or consists essentially of an amino acid sequence with at least 90%, preferably at least 91%, 92%, 93%, 94%, 95%, 96%, 97%, 98%, or 99%, sequence identity to an amino acid sequence represented by SEQ ID NO: 199 (SARS-2003, SARS_short_h), or SEQ ID NO: 201 (SARS-2003, SARS_short_i); and at least one is a MERS-COV spike glycoprotein fragment, wherein the MERS-COV fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 203 (MERS_Lytic_a), SEQ ID NO: 205 (MERS_Lytic_b), SEQ ID NO: 207 (MERS_Lytic_c), SEQ ID NO: 209 (MERS_Lytic_d), SEQ ID NO: 211 (MERS_Lytic_e), SEQ ID NO: 213 (MERS_Lytic_f), and SEQ ID NO: 215 (MERS_Lytic_g).

In some embodiments, the recombinant polypeptide comprises, consists of, or consists essentially of at least three immunogenic fragments, of which at least one is a SARS-CoV-2 spike glycoprotein fragment, wherein the SARS-COV-2 fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 245 (CTD_short_h) 2), SEQ ID NO: 249 (LS2435 Single domain_(GAMMA-Plus)), SEQ ID NO: 253 (BETA_CTD_short_i), SEQ ID NO: 257 (GAMMA_CTD_short_i), SEQ ID NO: 261 (GAMMA_CTD_short_i), SEQ ID NO: 265 (DELTA-Plus_CTD_short_i), SEQ ID NO: 269 (MU_CTD_short_i), SEQ ID NO: 273 (A.30_CTD_short_i), SEQ ID NO: 277 (DELTA-Plus+MU_CTD_short_i), SEQ ID NO: 281 (DELTA-Plus+A.30_CTD_short_i), SEQ ID NO: 285 (BETA_CTD_short_i)₂), SEQ ID NO: 289 ((GAMMA_CTD_short_i)₂), SEQ ID NO: 293 ((DELTA_CTD_short_i)₂), SEQ ID NO: 297 ((DELTA-Plus_CTD_short_i)₂), SEQ ID NO: 301 ((MU_CTD_short_i)₂), SEQ ID NO: 305 ((A.30_CTD_short_i)₂), SEQ ID NO: 309 ((DELTA-Plus+MU_CTD_short_i)₂), SEQ ID NO: 313 ((DELTA-Plus+A.30_CTD_short_i)₂), SEQ ID NO: 317 (Omicron_Boost_CTD_short_i), SEQ ID NO: 321 ((Omicron_Boost_CTD_short_i)₂), SEQ ID NO: 325 (Omicron_CTD_short_i), SEQ ID NO: 329 ((Omicron_CTD_short_i)₂), SEQ ID NO: 333 (OΔ+μ_1_CTD_short_i), SEQ ID NO: 337 ((OΔ+μ_1_CTD_short_i)₂), SEQ ID NO: 341 (OΔ+μ_2_CTD_short_i), SEQ ID NO: 345 ((OΔ+μ_2_CTD_short_i)₂), SEQ ID NO: 349 (OΔ+μ_3_CTD_short_i), SEQ ID NO: 353 ((Δ+μ_3_CTD_short_i)₂), SEQ ID NO: 357 (Δ+μ_4_CTD_short_i), SEQ ID NO: 361 ((OΔ+μ_4_CTD_short_i)₂), SEQ ID NO: 365 (CTD-gCd01), SEQ ID NO: 366 (CTD-gCd02), SEQ ID NO: 367 (CTD-gCd03), SEQ ID NO: 368 (CTD-gCd04), SEQ ID NO: 369 (CTD-gCd05), SEQ ID NO: 370 (CTD-gCd06), SEQ ID NO: 371 (CTD-gCd07), SEQ ID NO: 372 (CTD-gCd08), SEQ ID NO: 373 (CTD-gCd09), SEQ ID NO: 374 (CTD-gCd10), SEQ ID NO: 375 (CTD-gCd11), SEQ ID NO: 376 (CTD-gCd12), SEQ ID NO: 377 (CTD-gCd13), SEQ ID NO: 378 (CTD-gCd14), SEQ ID NO: 379 (CTD-gCd15), SEQ ID NO: 380 (CTD-gCd16), SEQ ID NO: 381 (CTD-gCd17), SEQ ID NO: 382 (CTD-gCd18), SEQ ID NO: 383 (CTD-gCd19), SEQ ID NO: 384 (CTD-gCd20), SEQ ID NO: 385 (CTD-gCd21), SEQ ID NO: 386 (CTD-gCd22), SEQ ID NO: 387 (CTD-gCd23), SEQ ID NO: 388 (CTD-gCd24), SEQ ID NO: 389 (CTD-gCd25), SEQ ID NO: 390 (CTD-gCd26), SEQ ID NO: 391 (CTD-gCd27), SEQ ID NO: 392 (CTD-gCd28), SEQ ID NO: 393 (CTD-gCd29), SEQ ID NO: 394 (CTD-gCd30), SEQ ID NO: 395 (CTD-gCd31), SEQ ID NO: 396 (CTD-gCd32), SEQ ID NO: 397 (CTD-gCd33), SEQ ID NO: 398 (CTD-gCd34), SEQ ID NO: 399 (CTD-gCd35), SEQ ID NO: 400 (CTD-gCd36), SEQ ID NO: 401 (CTD-gCd37), SEQ ID NO: 402 (CTD-gCd38), SEQ ID NO: 403 (CTD-gN01), SEQ ID NO: 404 (CTD-gN02), SEQ ID NO: 405 (CTD-gN03), SEQ ID NO: 406 (CTD-gN04), SEQ ID NO: 407 (CTD-gN05), SEQ ID NO: 408 (CTD-gN06), SEQ ID NO: 409 (CTD-gN07), SEQ ID NO: 410 (CTD-gN08), SEQ ID NO: 411 (CTD-gN09), SEQ ID NO: 412 (CTD-gN10), SEQ ID NO: 413 (CTD-gN11), SEQ ID NO: 414 (CTD-gN12), SEQ ID NO: 415 (CTD-gN13) SEQ ID NO: 416 (CTD-gN14), SEQ ID NO: 417 (CTD-gN15), SEQ ID NO: 418 (CTD-gN16), SEQ ID NO: 419 (CTD-gN17), SEQ ID NO: 420 (CTD-gN18), SEQ ID NO: 421 (CTD-gN19), SEQ ID NO: 422 (CTD-gN20), SEQ ID NO: 423 (CTD-gN21), SEQ ID NO: 424 (CTD-gN22), SEQ ID NO: 425 (CTD-gN23), SEQ ID NO: 426 (CTD-gN24), SEQ ID NO: 427 (CTD-gN25), SEQ ID NO: 428 (CTD-gN26), SEQ ID NO: 429 (CTD-gN27), SEQ ID NO: 430 (CTD-gN28), SEQ ID NO: 431 (CTD-gN29), SEQ ID NO: 432 (CTD-gN30), SEQ ID NO: 433 (CTD-gN31), SEQ ID NO: 434 (CTD-gN32), SEQ ID NO: 435 (CTD-gN33), SEQ ID NO: 436 (CTD-gN34), SEQ ID NO: 437 (CTD-gN35), SEQ ID NO: 438 (CTD-gN36), SEQ ID NO: 439 (CTD-gN37), SEQ ID NO: 440 (CTD-gN38), SEQ ID NO: 441 (CTD-gN39), SEQ ID NO: 442 (CTD-gN40), SEQ ID NO: 443 (CTD-gN41), SEQ ID NO: 444 (CTD-gN42), SEQ ID NO: 445 (CTD-gN43), SEQ ID NO: 446 (CTD-gN44), SEQ ID NO: 447 (CTD-gN45), SEQ ID NO: 448 (CTD-gN46), SEQ ID NO: 449 (CTD-gN47), and SEQ ID NOs: 705-716; at least one is a SARS-COV spike glycoprotein fragment, wherein the SARS-COV fragment comprises, consists of, or consists essentially of an amino acid sequence with at least 90%, preferably at least 91%, 92%, 93%, 94%, 95%, 96%, 97%, 98%, or 99%, sequence identity to an amino acid sequence represented by SEQ ID NO: 199 (SARS-2003, SARS_short_h), or SEQ ID NO: 201 (SARS-2003, SARS_short_i); and at least one is a MERS-COV spike glycoprotein fragment, wherein the MERS-COV fragment comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 203 (MERS_Lytic_a), SEQ ID NO: 205 (MERS_Lytic_b), SEQ ID NO: 207 (MERS_Lytic_c), SEQ ID NO: 209 (MERS_Lytic_d), SEQ ID NO: 211 (MERS_Lytic_e), SEQ ID NO: 213 (MERS_Lytic_f), and SEQ ID NO: 215 (MERS_Lytic_g), wherein the wherein the SARS-COV-2 fragment preferably comprises, consists of, or consists essentially of an amino acid sequence with at least 90%, preferably at least 91%, 92%, 93%, 94%, 95%, 96%, 97%, 98%, or 99%, sequence identity to an amino acid sequence selected from the group consisting of SEQ ID NOs: 277, 281, 309, 313, 333, and 337.

In some embodiments, the recombinant polypeptide includes at least two immunogenic fragments, wherein each of the at least two immunogenic fragments comprises, consists of, or consists essentially of, an amino acid sequence with at least 90%, preferably at least 91%, 92%, 93%, 94%, 95%, 96%, 97%, 98%, or 99%, sequence identity to an amino acid sequence independently selected from the group consisting of SEQ ID NO: 1 (CTD_long_a), SEQ ID NO: 3 (CTD_long_a_D614G), SEQ ID NO: 5 (CTD_long_a-Dimer), SEQ ID NO: 7 (CTD_long_b), SEQ ID NO: 9 (CTD_long_c), SEQ ID NO: 11 (CTD_long_d), SEQ ID NO: 13 (CTD_long_e), SEQ ID NO: 15 (CTD_long_f), SEQ ID NO: 17 (CTD_long_g), SEQ ID NO: 19 (CTD_long_h), SEQ ID NO: 21 (CTD_long_i), SEQ ID NO: 23 (CTD_short_a), SEQ ID NO: 25 (CTD_short_b), SEQ ID NO: 27 (CTD_short_c), SEQ ID NO: 29 (CTD_short_d), SEQ ID NO: 31 (CTD_short_e), SEQ ID NO: 33 (CTD_short_f), SEQ ID NO: 35 (CTD_short_g), SEQ ID NO: 37 (CTD_short_h), SEQ ID NO: 39 (CTD_short_i), SEQ ID NO: 41 (CTD_vs_a), SEQ ID NO: 43 (CTD_vs_b), SEQ ID NO: 45 (CTD_vs_c), SEQ ID NO: 47 (CTD_vs_d), SEQ ID NO: 49 (CTD_vs_e), SEQ ID NO: 51 (RBD_a), SEQ ID NO: 53 (RBD_b), SEQ ID NO: 55 (RBD_c), SEQ ID NO: 57 (RBD_d), SEQ ID NO: 59 (RBD_e), SEQ ID NO: 61 (NTD_long_a), SEQ ID NO: 63 (NTD_short_a), SEQ ID NO: 171 (RBD-tight), SEQ ID NO: 173 ((RBD-tight)₂), SEQ ID NO: 175 (RBD-extended), SEQ ID NO: 177 ((RBD-extended)₂), SEQ ID NO: 179 (RBD), SEQ ID NO: 181 ((RBD)₂), SEQ ID NO: 183 ((CTD_short_d)₂), SEQ ID NO: 185 ((CTD_short_i)₂), SEQ ID NO: 187 ((CTD_short_i)₂-mod. 1), SEQ ID NO: 189 ((CTD_short_i)₂-mod. 2), SEQ ID NO: 191 ((CTD_short_i)₂-mod. 3) and SEQ ID NOs: 705-716. In some embodiments, each of the at least two immunogenic fragments comprises, consists of, or consists essentially of an amino acid sequence with at least 90%, preferably at least 91%, 92%, 93%, 94%, 95%, 96%, 97%, 98%, or 99%, sequence identity to an amino acid sequence independently selected from the group consisting of SEQ ID NO: 183 ((CTD_short_d)₂), SEQ ID NO: 39 (CTD_short_i), SEQ ID NO: 29 (CTD_short_d), SEQ ID NO: 31 (CTD_short_e), SEQ ID NO: 37 (CTD_short_h), and SEQ ID NO: 23 (CTD_short_a). In some embodiments, each of the at least two immunogenic fragments comprises, consists of, or consists essentially of an amino acid sequence with at least 90%, preferably at least 91%, 92%, 93%, 94%, 95%, 96%, 97%, 98%, or 99%, sequence identity to the amino acid sequence of SEQ ID NO: 39 (CTD_short_i). In further embodiments, each of the at least two immunogenic fragments comprises, consists of, or consists essentially of an amino acid sequence independently selected from the group consisting of SEQ ID NO: 183 ((CTD_short_d)₂), SEQ ID NO: 39 (CTD_short_i), SEQ ID NO: 29 (CTD_short_d), SEQ ID NO: 31 (CTD_short_e), SEQ ID NO: 37 (CTD_short_h), and SEQ ID NO: 23 (CTD_short_a). In further embodiments, each of the at least two immunogenic fragments comprises, consists of, or consists essentially of the amino acid sequence of SEQ ID NO: 39 (CTD_short_i). In further embodiments, each of the at least two immunogenic fragments comprises, consists of, or consists essentially of an amino acid sequence independently selected from the group consisting of SEQ ID NOs: 705-716.

In some embodiments, the recombinant polypeptide includes at least two immunogenic fragments, wherein each of the at least two immunogenic fragments comprises, consists of, or consists essentially of, an amino acid sequence with at least 90%, preferably at least 91%, 92%, 93%, 94%, 95%, 96%, 97%, 98%, or 99%, sequence identity to an amino acid sequence independently selected from the group consisting of SEQ ID NO: 245 (CTD_short_h) 2), SEQ ID NO: 249 (LS2435 Single domain_(GAMMA-Plus)), SEQ ID NO: 253 (BETA_CTD_short_i), SEQ ID NO: 257 (GAMMA_CTD_short_i), SEQ ID NO: 261 (GAMMA_CTD_short_i), SEQ ID NO: 265 (DELTA-Plus_CTD_short_i), SEQ ID NO: 269 (MU_CTD_short_i), SEQ ID NO: 273 (A.30_CTD_short_i), SEQ ID NO: 277 (DELTA-Plus+MU_CTD_short_i), SEQ ID NO: 281 (DELTA-Plus+A.30_CTD_short_i), SEQ ID NO: 285 (BETA_CTD_short_i)₂), SEQ ID NO: 289 ((GAMMA_CTD_short_i)₂), SEQ ID NO: 293 ((DELTA_CTD_short_i)₂), SEQ ID NO: 297 ((DELTA-Plus_CTD_short_i)₂), SEQ ID NO: 301 ((MU_CTD_short_i)₂), SEQ ID NO: 305 ((A.30_CTD_short_i)₂), SEQ ID NO: 309 ((DELTA-Plus+MU_CTD_short_i)₂), SEQ ID NO: 313 ((DELTA-Plus+A.30_CTD_short_i)₂), SEQ ID NO: 317 (Omicron_Boost_CTD_short_i), SEQ ID NO: 321 ((Omicron_Boost_CTD_short_i)₂), SEQ ID NO: 325 (Omicron_CTD_short_i), SEQ ID NO: 329 ((Omicron_CTD_short_i)₂), SEQ ID NO: 333 (OΔ+μ_1_CTD_short_i), SEQ ID NO: 337 ((OΔ+μ_1_CTD_short_i)₂), SEQ ID NO: 341 (Δ+μ_2_CTD_short_i), SEQ ID NO: 345 ((Δ+μ_3_CTD_short_i)₂), SEQ ID NO: 349 (OΔ+μ_3_CTD_short_i), SEQ ID NO: 353 ((OΔ+μ_3_CTD_short_i)₂), SEQ ID NO: 357 (OΔ+μ_4_CTD_short_i), SEQ ID NO: 361 ((OΔ+μ_4_CTD_short_i)₂), SEQ ID NO: 365 (CTD-gCd01), SEQ ID NO: 366 (CTD-gCd02), SEQ ID NO: 367 (CTD-gCd03), SEQ ID NO: 368 (CTD-gCd04), SEQ ID NO: 369 (CTD-gCd05), SEQ ID NO: 370 (CTD-gCd06), SEQ ID NO: 371 (CTD-gCd07), SEQ ID NO: 372 (CTD-gCd08), SEQ ID NO: 373 (CTD-gCd09), SEQ ID NO: 374 (CTD-gCd10), SEQ ID NO: 375 (CTD-gCd11), SEQ ID NO: 376 (CTD-gCd12), SEQ ID NO: 377 (CTD-gCd13), SEQ ID NO: 378 (CTD-gCd14), SEQ ID NO: 379 (CTD-gCd15), SEQ ID NO: 380 (CTD-gCd16), SEQ ID NO: 381 (CTD-gCd17), SEQ ID NO: 382 (CTD-gCd18), SEQ ID NO: 383 (CTD-gCd19), SEQ ID NO: 384 (CTD-gCd20), SEQ ID NO: 385 (CTD-gCd21), SEQ ID NO: 386 (CTD-gCd22), SEQ ID NO: 387 (CTD-gCd23), SEQ ID NO: 388 (CTD-gCd24), SEQ ID NO: 389 (CTD-gCd25), SEQ ID NO: 390 (CTD-gCd26), SEQ ID NO: 391 (CTD-gCd27), SEQ ID NO: 392 (CTD-gCd28), SEQ ID NO: 393 (CTD-gCd29), SEQ ID NO: 394 (CTD-gCd30), SEQ ID NO: 395 (CTD-gCd31), SEQ ID NO: 396 (CTD-gCd32), SEQ ID NO: 397 (CTD-gCd33), SEQ ID NO: 398 (CTD-gCd34), SEQ ID NO: 399 (CTD-gCd35), SEQ ID NO: 400 (CTD-gCd36), SEQ ID NO: 401 (CTD-gCd37), SEQ ID NO: 402 (CTD-gCd38), SEQ ID NO: 403 (CTD-gN01), SEQ ID NO: 404 (CTD-gN02), SEQ ID NO: 405 (CTD-gN03), SEQ ID NO: 406 (CTD-gN04), SEQ ID NO: 407 (CTD-gN05), SEQ ID NO: 408 (CTD-gN06), SEQ ID NO: 409 (CTD-gN07), SEQ ID NO: 410 (CTD-gN08), SEQ ID NO: 411 (CTD-gN09), SEQ ID NO: 412 (CTD-gN10), SEQ ID NO: 413 (CTD-gN11), SEQ ID NO: 414 (CTD-gN12), SEQ ID NO: 415 (CTD-gN13) SEQ ID NO: 416 (CTD-gN14), SEQ ID NO: 417 (CTD-gN15), SEQ ID NO: 418 (CTD-gN16), SEQ ID NO: 419 (CTD-gN17), SEQ ID NO: 420 (CTD-gN18), SEQ ID NO: 421 (CTD-gN19), SEQ ID NO: 422 (CTD-gN20), SEQ ID NO: 423 (CTD-gN21), SEQ ID NO: 424 (CTD-gN22), SEQ ID NO: 425 (CTD-gN23), SEQ ID NO: 426 (CTD-gN24), SEQ ID NO: 427 (CTD-gN25), SEQ ID NO: 428 (CTD-gN26), SEQ ID NO: 429 (CTD-gN27), SEQ ID NO: 430 (CTD-gN28), SEQ ID NO: 431 (CTD-gN29), SEQ ID NO: 432 (CTD-gN30), SEQ ID NO: 433 (CTD-gN31), SEQ ID NO: 434 (CTD-gN32), SEQ ID NO: 435 (CTD-gN33), SEQ ID NO: 436 (CTD-gN34), SEQ ID NO: 437 (CTD-gN35), SEQ ID NO: 438 (CTD-gN36), SEQ ID NO: 439 (CTD-gN37), SEQ ID NO: 440 (CTD-gN38), SEQ ID NO: 441 (CTD-gN39), SEQ ID NO: 442 (CTD-gN40), SEQ ID NO: 443 (CTD-gN41), SEQ ID NO: 444 (CTD-gN42), SEQ ID NO: 445 (CTD-gN43), SEQ ID NO: 446 (CTD-gN44), SEQ ID NO: 447 (CTD-gN45), SEQ ID NO: 448 (CTD-gN46), SEQ ID NO: 449 (CTD-gN47), and SEQ ID NOs: 705-716.

In some embodiments, the recombinant polypeptide includes at least two immunogenic fragments, wherein each of the at least two immunogenic fragments comprises, consists of, or consists essentially of, an amino acid sequence with at least 90%, preferably at least 91%, 92%, 93%, 94%, 95%, 96%, 97%, 98%, or 99%, sequence identity to an amino acid sequence independently selected from the group consisting of SEQ ID NOs: 277, 281, 309, 313, 333, and 337.

Nucleotide or amino acid sequence “identity,” as referenced herein, can be determined by comparing a nucleotide or amino acid sequence of interest to a reference nucleotide or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min (TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244:332-350 (1994).

In some embodiments, the recombinant polypeptide includes a plurality of identical immunogenic fragments. For example, a recombinant polypeptide can include two, three, four, five, six, seven, eight, nine, ten, or more immunogenic fragments, wherein each fragment comprises, consists of, or consists essentially of the same amino acid sequence. In some embodiments, the recombinant polypeptide includes two, three, four or five identical immunogenic fragments. In yet further embodiments, the recombinant polypeptide includes two or three identical immunogenic fragments.

In some embodiments, the recombinant polypeptide includes a plurality of non-identical immunogenic fragments. For example, a recombinant polypeptide can include two, three, four, five, six, seven, eight, nine, ten, or more immunogenic fragments, wherein each fragment comprises, consists of, or consists essentially of a different amino acid sequence from each other fragment, i.e., each fragment is a different fragment. In some embodiments, the recombinant polypeptide includes two, three, four or five different immunogenic fragments. In yet further embodiments, the recombinant polypeptide includes two or three different immunogenic fragments.

In some embodiments, the recombinant polypeptide includes a plurality of immunogenic fragments, in which some of the fragments are identical, but not all. For example, a recombinant polypeptide can include two, three, four, five, six, seven, eight, nine, ten, or more immunogenic fragments, wherein each fragment comprises, consists of, or consists essentially of the same amino acid sequence, while also including one, two, three, four, five, six, seven, eight, nine, ten, or more immunogenic fragments, wherein each fragment comprises, consists of, or consists essentially of a different amino acid sequence from each other fragment. In some embodiments, the recombinant polypeptide includes a total of two, three, four or five immunogenic fragments. In yet further embodiments, the recombinant polypeptide includes a total of two or three immunogenic fragments.

Within the recombinant polypeptide, the at least one immunogenic fragment can be arranged in any suitable serial orientation with respect to the Fc region. In some embodiments, the at least one immunogenic fragment is connected to the N-terminus of the Fc region. This orientation can be depicted as [immunogenic fragment]_{X}—[N-terminus—Fc region—C-terminus], wherein X is an integer 1-10 representing the number of immunogenic fragments within the recombinant polypeptide. In some embodiments, the at least one immunogenic fragment is connected to the C-terminus of the Fc region. This orientation can be depicted as [N-terminus—Fc region—C-terminus]—[immunogenic fragment]_{X}, wherein X is an integer 1-10 representing the number of immunogenic fragments within the recombinant polypeptide. In some embodiments, wherein the recombinant polypeptide includes at least two immunogenic fragments, at least one immunogenic fragment is connected to the N-terminus of the Fc region, and at least one immunogenic fragment is connected to the C-terminus of the Fc region. This orientation can be depicted as [immunogenic fragment]x—[N-terminus—Fc region—C-terminus]—[immunogenic fragment]_{Y}, wherein X and Y are independently an integer 1-10 representing the number of immunogenic fragments connected to each side of the Fc region. In some embodiments, wherein the recombinant polypeptide includes two immunogenic fragments, both immunogenic fragments are connected to the N-terminus of the Fc region. This orientation can be depicted as [immunogenic fragment]—[immunogenic fragment]—[N-terminus-Fc region—C-terminus]. In other embodiments, wherein the recombinant polypeptide includes two immunogenic fragments, both immunogenic fragments are connected to the C-terminus of the Fc region. This orientation can be depicted as [N-terminus—Fc region—C-terminus]—[immunogenic fragment]—[immunogenic fragment]. In some embodiments, wherein the recombinant polypeptide includes three immunogenic fragments, each immunogenic fragment is connected to the N-terminus of the Fc region. This orientation can be depicted as [immunogenic fragment]—[immunogenic fragment]—[immunogenic fragment]—[N-terminus—Fc region—C-terminus]. In other embodiments, wherein the recombinant polypeptide includes three immunogenic fragments, each immunogenic fragment is connected to the C-terminus of the Fc region. This orientation can be depicted as [N-terminus—Fc region—C-terminus]—[immunogenic fragment]—[immunogenic fragment]—[immunogenic fragment].

Embodiments of the recombinant polypeptide that include a plurality of immunogenic fragments provide for a flexible expression platform with robust expression of full-length protein, modality to modify individual or multiple domains within one or more of the plurality of immunogenic fragments to reflect the most recent virus variant sequence(s), allow for single step affinity purification, and provide high-level, long-term immune response as tested in Rhesus macaques.

In some embodiments, wherein the recombinant polypeptide includes a plurality of immunogenic fragments, the immunogenic fragments are connected to each other via a linker. The linker can be any suitable linker. Suitable linkers include a polypeptide comprising, consisting of, or consisting essentially of, an amino acid sequence of 1-35 residues, wherein each residue is independently serine, glycine, or aspartic acid, and further wherein the amino acid sequence contains zero or one aspartic acid residues. Other suitable linkers include a polypeptide comprising an amino acid sequence selected from the group consisting of SEQ ID NO: 65 (Fc1), SEQ ID NO: 67 (Fc1-TEV), SEQ ID NO: 69 (Fc1-Rv3C), SEQ ID NO: 193 (Short), SEQ ID NO: 195 (Medium), and SEQ ID NO: 197 (Long). When a recombinant polypeptide includes two or more such linkers, the amino acid sequence of the linkers can be identical or different. In some embodiments, wherein the recombinant polypeptide includes a plurality of immunogenic fragments, the immunogenic fragments are connected directly to each other without an intervening linker.

In some embodiments, the one or more immunogenic fragments are connected to the antibody Fc region via a linker. The linker can be any suitable linker. Suitable linkers include a polypeptide comprising an amino acid sequence of 1-35 residues, wherein each residue is independently serine, glycine, or aspartic acid, and further wherein the amino acid sequence contains zero or one aspartic acid residues. Other suitable linkers include a polypeptide comprising an amino acid sequence selected from the group consisting of SEQ ID NO: 65 (Fc1), SEQ ID NO: 67 (Fc1-TEV), SEQ ID NO: 69 (Fc1-Rv3C), SEQ ID NO: 193 (Short), SEQ ID NO: 195 (Medium), and SEQ ID NO: 197 (Long). When a recombinant polypeptide includes two or more such linkers, the amino acid sequence of the linkers can be identical or different from each other. In some embodiments, wherein the recombinant polypeptide includes at least one immunogenic fragment connected to the N-terminus of the Fc region, and/or at least one immunogenic fragment is connected to the C-terminus of the Fc region, the immunogenic fragment(s) nearest to the Fc region are connected directly to the Fc region without an intervening linker.

The antibody Fc region included in the recombinant polypeptide can be any suitable antibody Fc region. Suitable antibody Fc regions include wild-type human or other animal Imunoglobulin Fc regions such as IgG, IgA, IgD, IgE, IgM, and their respective subclases, for example, human IgG1 Fc regions and Fc regions derived therefrom, as well as IgA Fc regions and Fc regions derived therefrom. Other suitable antibody Fc regions include mutant Fc regions that enhance or diminish Fc-receptor binding affinity to speed up or slow down uptake, respectively. In some embodiments, the antibody Fc region comprises the amino acid sequence of SEQ ID NO: 71.

In some embodiments, the recombinant polypeptide comprises, consists of, or consists essentially of, an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 73 (LS2330 [CTD_short_a-Fc]), SEQ ID NO: 75 (LS3472, LS3473, LS3474 [CTD_long_a-Fc]), SEQ ID NO: 77 (LS3477 [CTD_short_a-TEV-Fc]), SEQ ID NO: 79 (LS3485 [CTD_long_a TEV-Fc]), SEQ ID NO: 81 (LS3489 [CTD_short_a_Rv3c-Fc]), SEQ ID NO: 83 (LS3497 [CTD_long_a-Rv3c-Fc], SEQ ID NO: 85 (LS2316, LS2317, LS2318, LS2319 [CTD_long_a-His8]), SEQ ID NO: 87 (LS3479 [NTD_short_a-TEV-Fc]), SEQ ID NO: 89 (LS3475 [NTD_long_a-TEV-Fc]), SEQ ID NO: 91 (LS3491 [NTD_short_a-Rv3c-Fc]), SEQ ID NO: 93 (LS3487 [NTD_long_a-Rv3C-Fc]), SEQ ID NO: 95 (LS2326 [NTD_long_a-Fc]), SEQ ID NO: 97 (LS2354 [CTD_long_a_D614G-Fc]), SEQ ID NO: 99 (LS2355 [CTD_long_a-Dimer-Fc]), SEQ ID NO:101 (LS2356 [CTD_long_b-Fc]), SEQ ID NO: 103 (LS2357 [CTD_long_c-Fc]), SEQ ID NO: 105 (LS2358 [CTD_long_d-Fc]), SEQ ID NO: 107 (LS2359 [CTD_long_e-Fc]), SEQ ID NO: 109 (LS2360 [CTD_long_f-Fc]), SEQ ID NO: 111 (LS2361 [CTD_long_g-Fc]), SEQ ID NO: 113 (LS2362 [CTD_long_h-Fc]), SEQ ID NO: 115 (LS2363 [CTD_long_i-Fc]), SEQ ID NO: 117 (LS2364 [CTD_short_b-Fc]), SEQ ID NO: 119 (LS2365 [CTD_short_c-Fc]), SEQ ID NO: 121 (LS2366 [CTD_short_d-Fc]), SEQ ID NO: 123 (LS2367 [CTD_short_e-Fc]), SEQ ID NO: 125 (LS2368 [CTD_short_f-Fc]), SEQ ID NO: 127 (LS2369 [CTD_short_g-Fc]), SEQ ID NO: 129 (LS2370 [CTD_short_h-Fc]), SEQ ID NO: 131 (LS2371 [CTD_short_i-Fc]), SEQ ID NO: 133 (LS2372 [CTD_vs_a-Fc]), SEQ ID NO: 135 (LS2373 [CTD_vs_b-Fc]), SEQ ID NO: 137 (LS2374 [CTD_vs_c-Fc]), SEQ ID NO: 139 (LS2375 [CTD_vs_d-Fc]), SEQ ID NO: 141 (LS2376 [CTD_vs_e-Fc]), SEQ ID NO: 143 (LS2377 [RBD_a-Fc]), SEQ ID NO: 145 (LS2378 [RBD_b-Fc]), SEQ ID NO: 147 (LS2379 [RBD_c-Fc]), SEQ ID NO: 149 (LS2380 [RBD_d-Fc]), SEQ ID NO: 151 (LS2381 [RBD_e-Fc]), SEQ ID NO: 153 (LS2382 [NTD_short_a-Fc]), SEQ ID NO: 155 (LS2393 [(RBD-tight)₂-Fc]), SEQ ID NO: 157 (LS2394 [(RBD-extended)₂-Fc]), SEQ ID NO: 159 (LS2395 [(RBD)₂-Fc]), SEQ ID NO: 161 (LS2397-2400 [(CTD_short_d)₂-Fc]), SEQ ID NO: 163 (LS2401-2404 [(CTD_short_i)₂-Fc]), SEQ ID NO: 165 (LS2421 and LS2422 [(CTD_short_i)₂-Fc)-mod. 1], SEQ ID NO: 167 (LS2423 [(CTD_short_i)₂-Fc)-mod. 2], SEQ ID NO: 169 (LS2435 [(CTD_short_i)₂-Fc)-mod. 3], SEQ ID NO: 217, SEQ ID NO: 219, SEQ ID NO: 221 SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 237, SEQ ID NO: 239, SEQ ID NO: 241, SEQ ID NO: 243, and SEQ ID NOs: 705-716. In some embodiments, the recombinant polypeptide comprises, consists of, or consists essentially of, an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 163 (LS2401-2404 [(CTD_short_i)₂-Fc]), SEQ ID NO: 165 (LS2421 and LS2422 [(CTD_short_i)₂-Fc)-mod. 1], SEQ ID NO: 167 (LS2423 [(CTD_short_i)₂-Fc)-mod. 2], and SEQ ID NO: 169 (LS2435 [(CTD_short_i)₂-Fc)-mod. 3]. In some embodiments, the recombinant polypeptide comprises, consists of, or consists essentially of, an amino acid sequence with at least 90%, preferably at least 91%, 92%, 93%, 94%, 95%, 96%, 97%, 98%, or 99%, sequence identity to the amino acid sequence of SEQ ID NO: 163 (LS2401-2404 [(CTD_short_i)₂-Fc]). In some embodiments, the recombinant polypeptide comprises, consists of, or consists essentially of, an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 161 (LS2397-2400 [(CTD_short_d)₂-Fc]), SEQ ID NO: 121 (LS2366 [CTD_short_d-Fc]), SEQ ID NO: 123 (LS2367 [CTD_short_e-Fc]), SEQ ID NO: 129 (LS2370 [CTD_short_h-Fc]), SEQ ID NO: 131 (LS2371 [CTD_short_i-Fc]), and SEQ ID NO: 73 (LS2330 [CTD_short_a-Fc]). In another embodiment, the recombinant polypeptide comprises, consists of, or consists essentially of, an amino acid sequence with at least 90%, preferably at least 91%, 92%, 93%, 94%, 95%, 96%, 97%, 98%, or 99%, sequence identity to the amino acid sequence represented by SEQ ID NO: 163. In another embodiment, the recombinant polypeptide comprises, consists of, or consists essentially of, an amino acid sequence with at least 90%, preferably at least 91%, 92%, 93%, 94%, 95%, 96%, 97%, 98%, or 99%, sequence identity to the amino acid sequence represented by SEQ ID NO: 169.

In some embodiments, the recombinant polypeptide comprises, consists of, or consists essentially of, an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 247 (CTD_short_h) 2-Fc), SEQ ID NO: 251 (LS2435 Single domain_(GAMMA-Plus)-Fc), SEQ ID NO: 255 (BETA_CTD_short_i-Fc), SEQ ID NO: 259 (GAMMA_CTD_short_i-Fc), SEQ ID NO: 263 (GAMMA_CTD_short_i-Fc), SEQ ID NO: 267 (DELTA-Plus_CTD_short_i-Fc), SEQ ID NO: 271 (MU_CTD_short_i-Fc), SEQ ID NO: 275 (A.30_CTD_short_i-Fc), SEQ ID NO: 279 (DELTA-Plus+MU_CTD_short_i-Fc), SEQ ID NO: 283 (DELTA-Plus+A.30_CTD_short_i-Fc), SEQ ID NO: 287 (BETA_CTD_short_i)₂-Fc), SEQ ID NO: 291 ((GAMMA_CTD_short_i)₂-Fc), SEQ ID NO: 295 ((DELTA_CTD_short_i)₂-Fc), SEQ ID NO: 299 ((DELTA-Plus_CTD_short_i)₂-Fc), SEQ ID NO: 303 ((MU_CTD_short_i)₂-Fc), SEQ ID NO: 307 ((A.30_CTD_short_i)₂-Fc), SEQ ID NO: 311 ((DELTA-Plus+MU_CTD_short_i)₂-Fc), SEQ ID NO: 315 ((DELTA-Plus+A.30_CTD_short_i)₂-Fc), SEQ ID NO: 319 (Omicron_Boost_CTD_short_i-Fc), SEQ ID NO: 323 ((Omicron_Boost_CTD_short_i)₂-Fc), SEQ ID NO: 327 (Omicron_CTD_short_i-Fc), SEQ ID NO: 331 ((Omicron_CTD_short_i)₂-Fc), SEQ ID NO: 335 (OΔ+μ_1_CTD_short_i-Fc), SEQ ID NO: 339 ((OΔ+μ_1_CTD_short_i)₂-Fc), SEQ ID NO: 343 (OΔ+μ_2_CTD_short_i-Fc), SEQ ID NO: 347 ((OΔ+μ_2_CTD_short_i)₂-Fc), SEQ ID NO: 351 (OΔ+μ_3_CTD_short_i-Fc), SEQ ID NO: 355 ((Δ+μ_3_CTD_short_i)₂-Fc), SEQ ID NO: 359 (OΔ+μ_4_CTD_short_i-Fc), SEQ ID NO: 363 ((OΔ+μ_4_CTD_short_i)₂-Fc), SEQ ID NO: 535 (CTD-gCd01-Fc), SEQ ID NO: 536 (CTD-gCd02-Fc), SEQ ID NO: 537 (CTD-gCd03-Fc), SEQ ID NO: 538 (CTD-gCd04-Fc), SEQ ID NO: 539 (CTD-gCd05-Fc), SEQ ID NO: 540 (CTD-gCd06-Fc), SEQ ID NO: 541 (CTD-gCd07-Fc), SEQ ID NO: 542 (CTD-gCd08-Fc), SEQ ID NO: 543 (CTD-gCd09-Fc), SEQ ID NO: 544 (CTD-gCd10-Fc), SEQ ID NO: 545 (CTD-gCd11-Fc), SEQ ID NO: 546 (CTD-gCd12-Fc), SEQ ID NO: 547 (CTD-gCd13-Fc), SEQ ID NO: 548 (CTD-gCd14-Fc), SEQ ID NO: 549 (CTD-gCd15-Fc), SEQ ID NO: 550 (CTD-gCd16-Fc), SEQ ID NO: 551 (CTD-gCd17-Fc), SEQ ID NO: 552 (CTD-gCd18-Fc), SEQ ID NO: 553 (CTD-gCd19-Fc), SEQ ID NO: 554 (CTD-gCd20-Fc), SEQ ID NO: 555 (CTD-gCd21-Fc), SEQ ID NO: 556 (CTD-gCd22-Fc), SEQ ID NO: 557 (CTD-gCd23-Fc), SEQ ID NO: 558 (CTD-gCd24-Fc), SEQ ID NO: 559 (CTD-gCd25-Fc), SEQ ID NO: 560 (CTD-gCd26-Fc), SEQ ID NO: 561 (CTD-gCd27-Fc), SEQ ID NO: 562 (CTD-gCd28-Fc), SEQ ID NO: 563 (CTD-gCd29-Fc), SEQ ID NO: 564 (CTD-gCd30-Fc), SEQ ID NO: 565 (CTD-gCd31-Fc), SEQ ID NO: 566 (CTD-gCd32-Fc), SEQ ID NO: 567 (CTD-gCd33-Fc), SEQ ID NO: 568 (CTD-gCd34-Fc), SEQ ID NO: 569 (CTD-gCd35-Fc), SEQ ID NO: 570 (CTD-gCd36-Fc), SEQ ID NO: 571 (CTD-gCd37-Fc), SEQ ID NO: 572 (CTD-gCd38-Fc), SEQ ID NO: 573 (CTD-gN01-Fc), SEQ ID NO: 574 (CTD-gN02), SEQ ID NO: 575 (CTD-gN03-Fc), SEQ ID NO: 576 (CTD-gN04-Fc), SEQ ID NO: 577 (CTD-gN05-Fc), SEQ ID NO: 578 (CTD-gN06-Fc), SEQ ID NO: 579 (CTD-gN07-Fc), SEQ ID NO: 580 (CTD-gN08-Fc), SEQ ID NO: 581 (CTD-gN09-Fc), SEQ ID NO: 582 (CTD-gN10-Fc), SEQ ID NO: 583 (CTD-gN11-Fc), SEQ ID NO: 584 (CTD-gN12-Fc), SEQ ID NO: 585 (CTD-gN13-Fc) SEQ ID NO: 586 (CTD-gN14-Fc), SEQ ID NO: 587 (CTD-gN15-Fc), SEQ ID NO: 588 (CTD-gN16-Fc), SEQ ID NO: 589 (CTD-gN17-Fc), SEQ ID NO: 590 (CTD-gN18-Fc), SEQ ID NO: 591 (CTD-gN19-Fc), SEQ ID NO: 592 (CTD-gN20-Fc), SEQ ID NO: 593 (CTD-gN21-Fc), SEQ ID NO: 594 (CTD-gN22-Fc), SEQ ID NO: 595 (CTD-gN23-Fc), SEQ ID NO: 596 (CTD-gN24-Fc), SEQ ID NO: 597 (CTD-gN25-Fc), SEQ ID NO: 598 (CTD-gN26-Fc), SEQ ID NO: 599 (CTD-gN27-Fc), SEQ ID NO: 600 (CTD-gN28-Fc), SEQ ID NO: 601 (CTD-gN29-Fc), SEQ ID NO: 602 (CTD-gN30-Fc), SEQ ID NO: 603 (CTD-gN31-Fc), SEQ ID NO: 604 (CTD-gN32-Fc), SEQ ID NO: 605 (CTD-gN33-Fc), SEQ ID NO: 606 (CTD-gN34-Fc), SEQ ID NO: 607 (CTD-gN35-Fc), SEQ ID NO: 608 (CTD-gN36-Fc), SEQ ID NO: 609 (CTD-gN37-Fc), SEQ ID NO: 610 (CTD-gN38-Fc), SEQ ID NO: 611 (CTD-gN39-Fc), SEQ ID NO: 612 (CTD-gN40-Fc), SEQ ID NO: 613 (CTD-gN41-Fc), SEQ ID NO: 614 (CTD-gN42-Fc), SEQ ID NO: 615 (CTD-gN43-Fc), SEQ ID NO: 616 (CTD-gN44-Fc), SEQ ID NO: 617 (CTD-gN45-Fc), SEQ ID NO: 618 (CTD-gN46-Fc), SEQ ID NO: 619 (CTD-gN47), and SEQ ID NOs: 705-716.

In some embodiments, the recombinant polypeptide comprises, consists of, or consists essentially of, an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 279 (DELTA-Plus+MU_CTD_short_i-Fc), SEQ ID NO: 283 (DELTA-Plus+A.30_CTD_short_i-Fc), SEQ ID NO: 311 ((DELTA-Plus+MU_CTD_short_i)₂-Fc), SEQ ID NO: 315 ((DELTA-Plus+A.30_CTD_short_i)₂-Fc), SEQ ID NO: 335 (OΔ+μ_1_CTD_short_i-Fc), and SEQ ID NO: 339 ((OΔ+μ_1_CTD_short_i)₂-Fc).

In some embodiments, the recombinant polypeptide comprises, consists of, or consists essentially of, an amino acid sequence selected from the group consisting of SEQ ID NOS: 73, 75, 77, 79, 81, 83, 85, 87, 89, 91, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 217, 219, 221, 223, 225, 227, 229, 231, 233, 235, 237, 239, 241, 243, and 705-716. In some embodiments, the recombinant polypeptide comprises, consists of, or consists essentially of, an amino acid sequence selected from the group consisting of SEQ ID NO: 163 (LS2401-2404 [(CTD_short_i)₂-Fc]), SEQ ID NO: 165 (LS2421 and LS2422 [(CTD_short_i)₂-Fc), SEQ ID NO: 167 (LS2423 [(CTD_short_i)₂-Fc), and SEQ ID NO: 169 (LS2435 [(CTD_short_i)₂-Fc). In some embodiments, the recombinant polypeptide comprises, consists of, or consists essentially of, the amino acid sequence of SEQ ID NO: 163 (LS2401-2404 [(CTD_short_i)₂-Fc]). In some embodiments, the recombinant polypeptide comprises, consists of, or consists essentially of, an amino acid sequence selected from the group consisting of SEQ ID NO: 161 (LS2397-2400 [(CTD_short_d)₂-Fc]), SEQ ID NO: 121 (LS2366 [CTD_short_d-Fc]), SEQ ID NO: 123 (LS2367 [CTD_short_e-Fc]), SEQ ID NO: 129 (LS2370 [CTD_short_h-Fc]), SEQ ID NO: 131 (LS2371 [CTD_short_i-Fc]), and SEQ ID NO: 73 (LS2330 [CTD_short_a-Fc]). In another embodiment, the recombinant polypeptide comprises, consists of, or consists essentially of, the amino acid sequence represented by SEQ ID NO: 163. In another embodiment, the recombinant polypeptide comprises, consists of, or consists essentially of, the amino acid sequence represented by SEQ ID NO: 169. In some embodiments, the recombinant polypeptide comprises, consists of, or consists essentially of, an amino acid sequence selected from the group consisting of SEQ ID NOs: 705-716.

In some embodiments, the recombinant polypeptide comprises, consists of, or consists essentially of, an amino acid sequence selected from the group consisting of SEQ ID NOS: 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 535-619, and 705-716. In some embodiments, the recombinant polypeptide comprises, consists of, or consists essentially of, an amino acid sequence selected from the group consisting of SEQ ID NOs: 279, 283, 311, 315, 335, and 339.

In some embodiments, the recombinant polypeptide is encoded by a recombinant polynucleotide. In certain embodiments, the polynucleotide comprises, consists of, or consists essentially of a nucleic acid sequence with at least 90%, preferably at least 91%, 92%, 93%, 94%, 95%, 96%, 97%, 98%, or 99%, sequence identity to a nucleic acid sequence selected from the group consisting of SEQ ID NO: 74 (LS2330 [CTD_short_a-Fc]), SEQ ID NO: 76 (LS3472, LS3473, LS3474 [CTD_long_a-Fc]), SEQ ID NO: 78 (LS3477 [CTD_short_a-TEV-Fc]), SEQ ID NO: 80 (LS3485 [CTD_long_a TEV-Fc]), SEQ ID NO: 82 (LS3489 [CTD_short_a_Rv3c-Fc]), SEQ ID NO: 84 (LS3497 [CTD_long_a-Rv3c-Fc], SEQ ID NO: 86 (LS2316, LS2317, LS2318, LS2319 [CTD_long_a-His8]), SEQ ID NO: 88 (LS3479 [NTD_short_a-TEV-Fc]), SEQ ID NO: 90 (LS3475 [NTD_long_a-TEV-Fc]), SEQ ID NO: 92 (LS3491 [NTD_short_a-Rv3c-Fc]), SEQ ID NO: 94 (LS3487 [NTD_long_a-Rv3C-Fc]), SEQ ID NO: 96 (LS2326 [NTD_long_a-Fc]), SEQ ID NO: 98 (LS2354 [CTD_long_a_D614G-Fc]), SEQ ID NO: 100 (LS2355 [CTD_long_a-Dimer-Fc]), SEQ ID NO: 102 (LS2356 [CTD_long_b-Fc]), SEQ ID NO: 104 (LS2357 [CTD_long_c-Fc]), SEQ ID NO: 106 (LS2358 [CTD_long_d-Fc]), SEQ ID NO: 108 (LS2359 [CTD_long_e-Fc]), SEQ ID NO: 110 (LS2360 [CTD_long_f-Fc]), SEQ ID NO: 112 (LS2361 [CTD_long_g-Fc]), SEQ ID NO: 114 (LS2362 [CTD_long_h-Fc]), SEQ ID NO: 116 (LS2363 [CTD_long_i-Fc]), SEQ ID NO: 118 (LS2364 [CTD_short_b-Fc]), SEQ ID NO: 120 (LS2365 [CTD_short_c-Fc]), SEQ ID NO: 122 (LS2366 [CTD_short_d-Fc]), SEQ ID NO: 124 (LS2367 [CTD_short_e-Fc]), SEQ ID NO: 126 (LS2368 [CTD_short_f-Fc]), SEQ ID NO: 128 (LS2369 [CTD_short_g-Fc]), SEQ ID NO: 130 (LS2370 [CTD_short_h-Fc]), SEQ ID NO: 132 (LS2371 [CTD_short_i-Fc]), SEQ ID NO: 134 (LS2372 [CTD_vs_a-Fc]), SEQ ID NO: 136 (LS2373 [CTD_vs_b-Fc]), SEQ ID NO: 138 (LS2374 [CTD_vs_c-Fc]), SEQ ID NO: 140 (LS2375 [CTD_vs_d-Fc]), SEQ ID NO: 142 (LS2376 [CTD_vs_e-Fc]), SEQ ID NO: 144 (LS2377 [RBD_a-Fc]), SEQ ID NO: 146 (LS2378 [RBD_b-Fc]), SEQ ID NO: 148 (LS2379 [RBD_c-Fc]), SEQ ID NO: 150 (LS2380 [RBD_d-Fc]), SEQ ID NO: 152 (LS2381 [RBD_e-Fc]), SEQ ID NO: 154 (LS2382 [NTD_short_a-Fc]), SEQ ID NO: 156 (LS2393 [(RBD-tight)₂-Fc]), SEQ ID NO: 158 (LS2394 [(RBD-extended)₂-Fc]), SEQ ID NO: 160 (LS2395 [(RBD)₂-Fc]), SEQ ID NO: 162 (LS2397-2400 [(CTD_short_d)₂-Fc]), SEQ ID NO: 164 (LS2401-2404 [(CTD_short_i)₂-Fc]), SEQ ID NO: 166 (LS2421 and LS2422 [(CTD_short_i)₂-Fc), SEQ ID NO: 168 (LS2423 [(CTD_short_i)₂-Fc), SEQ ID NO: 170 (LS2435 [(CTD_short_i)₂-Fc), SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, and SEQ ID NOs: 717-728.

In some embodiments, the polynucleotide comprises, consists of, or consists essentially of a nucleic acid sequence with at least 90%, preferably at least 91%, 92%, 93%, 94%, 95%, 96%, 97%, 98%, or 99%, sequence identity to a nucleic acid sequence selected from the group consisting of SEQ ID NO: 164 (LS2401-2404 [(CTD_short_i)₂-Fc]), SEQ ID NO: 166 (LS2421 and LS2422 [(CTD_short_i)₂-Fc)-mod.1], SEQ ID NO: 168 (LS2423 [(CTD_short_i)₂-Fc)-mod. 2]), and SEQ ID NO: 170 (LS2435 [(CTD_short_i)₂-Fc-mod. 3). In some embodiments, the polynucleotide comprises, consists of, or consists essentially of, an nucleic acid sequence with at least 90%, preferably at least 91%, 92%, 93%, 94%, 95%, 96%, 97%, 98%, or 99%, sequence identity to the nucleic acid sequence of SEQ ID NO: 164 (LS2401-2404 [(CTD_short_i)₂-Fc]). In some embodiments, the polynucleotide comprises, consists of, or consists essentially of, an nucleic acid sequence with at least 90%, preferably at least 91%, 92%, 93%, 94%, 95%, 96%, 97%, 98%, or 99%, sequence identity to an nucleic acid sequence selected from the group consisting of SEQ ID NO: 162 (LS2397-2400 [(CTD_short_d)₂-Fc]), SEQ ID NO: 122 (LS2366 [CTD_short_d-Fc]), SEQ ID NO: 124 (LS2367 [CTD_short_e-Fc]), SEQ ID NO: 130 (LS2370 [CTD_short_h-Fc]), SEQ ID NO: 132 (LS2371 [CTD_short_i-Fc]), and SEQ ID NO: 74 (LS2330 [CTD_short_a-Fc]). In an embodiment, the polynucleotide comprises, consists of, or consists essentially of a nucleic acid sequence with at least 90%, preferably at least 91%, 92%, 93%, 94%, 95%, 96%, 97%, 98%, or 99%, sequence identity to the nucleic acid sequence represented by SEQ ID NO: 164. In an embodiment, the polynucleotide comprises, consists of, or consists essentially of a nucleic acid sequence with at least 90%, preferably at least 91%, 92%, 93%, 94%, 95%, 96%, 97%, 98%, or 99%, sequence identity to the nucleic acid sequence represented by SEQ ID NO: 170. In some embodiments, the polynucleotide comprises, consists of, or consists essentially of, an nucleic acid sequence with at least 90%, preferably at least 91%, 92%, 93%, 94%, 95%, 96%, 97%, 98%, or 99%, sequence identity to an nucleic acid sequence selected from the group consisting of SEQ ID NOs: 717-728.

In some embodiments, the recombinant polypeptide is encoded by a recombinant polynucleotide. In certain embodiments, the polynucleotide comprises, consists of, or consists essentially of a nucleic acid sequence with at least 90%, preferably at least 91%, 92%, 93%, 94%, 95%, 96%, 97%, 98%, or 99%, sequence identity to a nucleic acid sequence selected from the group consisting of SEQ ID NO: 248 (CTD_short_h) 2-Fc), SEQ ID NO: 252 (LS2435 Single domain_(GAMMA-Plus)-Fc), SEQ ID NO: 256 (BETA_CTD_short_i-Fc), SEQ ID NO: 260 (GAMMA_CTD_short_i-Fc), SEQ ID NO: 264 (GAMMA_CTD_short_i-Fc), SEQ ID NO: 268 (DELTA-Plus_CTD_short_i-Fc), SEQ ID NO: 272 (MU_CTD_short_i-Fc), SEQ ID NO: 276 (A.30_CTD_short_i-Fc), SEQ ID NO: 280 (DELTA-Plus+MU_CTD_short_i-Fc), SEQ ID NO: 284 (DELTA-Plus+A.30_CTD_short_i-Fc), SEQ ID NO: 288 (BETA_CTD_short_i)₂-Fc), SEQ ID NO: 292 ((GAMMA_CTD_short_i)₂-Fc), SEQ ID NO: 296 ((DELTA_CTD_short_i)₂-Fc), SEQ ID NO: 300 ((DELTA-Plus_CTD_short_i)₂-Fc), SEQ ID NO: 304 ((MU_CTD_short_i)₂-Fc), SEQ ID NO: 308 ((A.30_CTD_short_i)₂-Fc), SEQ ID NO: 312 ((DELTA-Plus+MU_CTD_short_i)₂-Fc), SEQ ID NO: 316 ((DELTA-Plus+A.30_CTD_short_i)₂-Fc), SEQ ID NO: 320 (Omicron_Boost_CTD_short_i-Fc), SEQ ID NO: 324 ((Omicron_Boost_CTD short_i)₂-Fc), SEQ ID NO: 328 (Omicron_CTD_short_i-Fc), SEQ ID NO: 332 ((Omicron_CTD short_i)₂-Fc), SEQ ID NO: 336 (OΔ+μ_1_CTD_short_i-Fc), SEQ ID NO: 340 ((Δ+μ_1_CTD_short_i)₂-Fc), SEQ ID NO: 344 (Δ+μ_2_CTD_short_i-Fc), SEQ ID NO: 348 ((OΔ+μ_2_CTD_short_i)₂-Fc), SEQ ID NO: 352 (OΔ+μ_3_CTD_short_i-Fc), SEQ ID NO: 356 ((Δ+μ_3_CTD_short_i)₂-Fc), SEQ ID NO: 360 (Δ+μ_4_CTD_short_i-Fc), SEQ ID NO: 364 ((OΔ+μ_4_CTD_short_i)₂-Fc), SEQ ID NO: 620 (CTD-gCd01-Fc), SEQ ID NO: 621 (CTD-gCd02-Fc), SEQ ID NO: 622 (CTD-gCd03-Fc), SEQ ID NO: 623 (CTD-gCd04-Fc), SEQ ID NO: 624 (CTD-gCd05-Fc), SEQ ID NO: 625 (CTD-gCd06-Fc), SEQ ID NO: 626 (CTD-gCd07-Fc), SEQ ID NO: 627 (CTD-gCd08-Fc), SEQ ID NO: 628 (CTD-gCd09-Fc), SEQ ID NO: 629 (CTD-gCd10-Fc), SEQ ID NO: 630 (CTD-gCd11-Fc), SEQ ID NO: 631 (CTD-gCd12-Fc), SEQ ID NO: 632 (CTD-gCd13-Fc), SEQ ID NO: 633 (CTD-gCd14-Fc), SEQ ID NO: 634 (CTD-gCd15-Fc), SEQ ID NO: 635 (CTD-gCd16-Fc), SEQ ID NO: 636 (CTD-gCd17-Fc), SEQ ID NO: 637 (CTD-gCd18-Fc), SEQ ID NO: 638 (CTD-gCd19-Fc), SEQ ID NO: 639 (CTD-gCd20-Fc), SEQ ID NO: 640 (CTD-gCd21-Fc), SEQ ID NO: 641 (CTD-gCd22-Fc), SEQ ID NO: 642 (CTD-gCd23-Fc), SEQ ID NO: 643 (CTD-gCd24-Fc), SEQ ID NO: 644 (CTD-gCd25-Fc), SEQ ID NO: 645 (CTD-gCd26-Fc), SEQ ID NO: 646 (CTD-gCd27-Fc), SEQ ID NO: 647 (CTD-gCd28-Fc), SEQ ID NO: 648 (CTD-gCd29-Fc), SEQ ID NO: 649 (CTD-gCd30-Fc), SEQ ID NO: 650 (CTD-gCd31-Fc), SEQ ID NO: 651 (CTD-gCd32-Fc), SEQ ID NO: 652 (CTD-gCd33-Fc), SEQ ID NO: 653 (CTD-gCd34-Fc), SEQ ID NO: 654 (CTD-gCd35-Fc), SEQ ID NO: 655 (CTD-gCd36-Fc), SEQ ID NO: 656 (CTD-gCd37-Fc), SEQ ID NO: 657 (CTD-gCd38-Fc), SEQ ID NO: 658 (CTD-gN01-Fc), SEQ ID NO: 659 (CTD-gN02), SEQ ID NO: 660 (CTD-gN03-Fc), SEQ ID NO: 661 (CTD-gN04-Fc), SEQ ID NO: 662 (CTD-gN05-Fc), SEQ ID NO: 663 (CTD-gN06-Fc), SEQ ID NO: 664 (CTD-gN07-Fc), SEQ ID NO: 665 (CTD-gN08-Fc), SEQ ID NO: 666 (CTD-gN09-Fc), SEQ ID NO: 667 (CTD-gN10-Fc), SEQ ID NO: 668 (CTD-gN11-Fc), SEQ ID NO: 669 (CTD-gN12-Fc), SEQ ID NO: 670 (CTD-gN13-Fc) SEQ ID NO: 671 (CTD-gN14-Fc), SEQ ID NO: 672 (CTD-gN15-Fc), SEQ ID NO: 673 (CTD-gN16-Fc), SEQ ID NO: 674 (CTD-gN17-Fc), SEQ ID NO: 675 (CTD-gN18-Fc), SEQ ID NO: 676 (CTD-gN19-Fc), SEQ ID NO: 677 (CTD-gN20-Fc), SEQ ID NO: 678 (CTD-gN21-Fc), SEQ ID NO: 679 (CTD-gN22-Fc), SEQ ID NO: 680 (CTD-gN23-Fc), SEQ ID NO: 681 (CTD-gN24-Fc), SEQ ID NO: 682 (CTD-gN25-Fc), SEQ ID NO: 683 (CTD-gN26-Fc), SEQ ID NO: 684 (CTD-gN27-Fc), SEQ ID NO: 685 (CTD-gN28-Fc), SEQ ID NO: 686 (CTD-gN29-Fc), SEQ ID NO: 687 (CTD-gN30-Fc), SEQ ID NO: 688 (CTD-gN31-Fc), SEQ ID NO: 689 (CTD-gN32-Fc), SEQ ID NO: 690 (CTD-gN33-Fc), SEQ ID NO: 691 (CTD-gN34-Fc), SEQ ID NO: 692 (CTD-gN35-Fc), SEQ ID NO: 693 (CTD-gN36-Fc), SEQ ID NO: 694 (CTD-gN37-Fc), SEQ ID NO: 695 (CTD-gN38-Fc), SEQ ID NO: 696 (CTD-gN39-Fc), SEQ ID NO: 697 (CTD-gN40-Fc), SEQ ID NO: 698 (CTD-gN41-Fc), SEQ ID NO: 699 (CTD-gN42-Fc), SEQ ID NO: 700 (CTD-gN43-Fc), SEQ ID NO: 701 (CTD-gN44-Fc), SEQ ID NO: 702 (CTD-gN45-Fc), SEQ ID NO: 703 (CTD-gN46-Fc), and SEQ ID NO: 704 (CTD-gN47).

In certain embodiments, the polynucleotide comprises, consists of, or consists essentially of a nucleic acid sequence with at least 90%, preferably at least 91%, 92%, 93%, 94%, 95%, 96%, 97%, 98%, or 99%, sequence identity to a nucleic acid sequence selected from the group consisting of SEQ ID NO: 280, 284, 312, 316, 336, and 340.

In some embodiments, the polynucleotide comprises, consists of, or consists essentially of a nucleic acid sequence selected from the group consisting of SEQ ID NO: 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 218, 220, 222, 224, 226, 228, 230, 232, 234, 236, 238, 240, 242, 244, and 717-728. In some embodiments, the polynucleotide comprises, consists of, or consists essentially of a nucleic acid sequence selected from the group consisting of SEQ ID NO: 164 (LS2401-2404 [(CTD_short_i)₂-Fc]), SEQ ID NO: 166 (LS2421 and LS2422 [(CTD_short_i)₂-Fc)-mod. 1], SEQ ID NO: 168 (LS2423 [(CTD_short_i)₂-Fc)-mod.2], and SEQ ID NO: 170 (LS2435 [(CTD_short_i)₂-Fc)-mod. 3]. In some embodiments, the polynucleotide comprises, consists of, or consists essentially of, the nucleic acid sequence of SEQ ID NO: 164 (LS2401-2404 [(CTD_short_i)₂-Fc]). In some embodiments, the polynucleotide comprises, consists of, or consists essentially of, an nucleic acid sequence selected from the group consisting of SEQ ID NO: 162 (LS2397-2400 [(CTD_short_d)₂-Fc]), SEQ ID NO: 122 (LS2366 [CTD_short_d-Fc]), SEQ ID NO: 124 (LS2367 [CTD_short_e-Fc]), SEQ ID NO: 130 (LS2370 [CTD_short_h-Fc]), SEQ ID NO: 132 (LS2371 [CTD_short_i-Fc]), and SEQ ID NO: 74 (LS2330 [CTD_short_a-Fc]). In an embodiment, the polynucleotide comprises, consists of, or consists essentially of a nucleic acid sequence represented by SEQ ID NO: 164. In an embodiment, the polynucleotide comprises, consists of, or consists essentially of a nucleic acid sequence represented by SEQ ID NO: 170. In some embodiments, the polynucleotide comprises, consists of, or consists essentially of, a nucleic acid sequence selected from the group consisting of SEQ ID NOs: 717-728.

In some embodiments, the polynucleotide comprises, consists of, or consists essentially of a nucleic acid sequence selected from the group consisting of SEQ ID NOs: 248, 252, 256, 260, 264, 268, 272, 276, 280, 284, 288, 292, 296, 300, 304, 308, 312, 316, 320, 324, 328, 332, 336, 340, 344, 348, 352, 356, 360, 364 620-704, and 717-728. In some embodiments, the polynucleotide comprises, consists of, or consists essentially of a nucleic acid sequence selected from the group consisting of SEQ ID NOs: 280, 284, 312, 316, 336, and 340.

In some embodiments, the nucleotide sequence of the polynucleotide is codon optimized and/or codon pair optimized.

Another embodiment is a recombinant vector that comprises, consists of, or consists essentially of, a polynucleotide that encodes the recombinant polypeptide described herein. The recombinant vector can be any suitable vector. Examples of suitable recombinant vectors include but are not limited to a pcDNA3.1, a pSV, a pCMV, a pBApo-CMV, or a pBApo-EF1alpha expression vector.

Yet another embodiment is an isolated cell that includes the recombinant polypeptide described herein or a recombinant polynucleotide that contains a nucleic acid sequence that encodes the recombinant polypeptide.

Another embodiment is a pharmaceutical composition that contains the recombinant polypeptide described herein and at least one pharmaceutically acceptable carrier. The at least one pharmaceutically acceptable carrier can be any suitable carrier. Examples of suitable carriers include water and any suitable buffer. Suitable buffers include HEPES-buffered saline and phosphate-buffered saline. In certain embodiments, the pharmaceutical composition further contains at least one adjuvant. The at least one adjuvant can be any suitable adjuvant. Examples of suitable adjuvants include alum adjuvants, emulsion adjuvants, and pattern recognition receptor agonist adjuvants. Further examples include AS03, MF59, Squalene Emulsion, Alum, aluminum hydroxide gels, calcium phosphate hydroxide, paraffin oil, cytokines (IL-1, IL-2, IL-12), killed bacterial products such as Bordetella and Mycobacterium bacteria, bacterial toxoids, squalene and DL-α-tocopherol emulsions, squalene-oil-in-water emulsion, aluminum phosphate gels, saponins, cyclic dinucleotides, and TLR agonists, preferably TLR1, TLR2, TLR4, TLR5, TLR7, TLR8, TLR9 etc., and combinations thereof. In certain embodiments, the adjuvant is AS03. In certain other embodiments, the adjuvant is Alum. In yet further embodiments, the pharmaceutical composition does not contain an adjuvant.

The pharmaceutical composition can contain any therapeutically effective amount of the recombinant polypeptide described herein. A therapeutically effective amount is an amount sufficient to induce an immune response against the target virus or viruses, for instance, SARS-CoV-2, SARS-COV, and/or MERS-COV, preferably SARS-COV-2. Typically, a dosage is therapeutically effective if it prevents, inhibits, reduces, eliminates, protects against, and/or delays the onset of an infection or an infectious clinical condition caused by a beta coronavirus in a subject. Infectious clinical conditions include, for example, fever or chills, cough, shortness of breath or difficult breathing, fatigue, muscle or body aches, headache, loss of taste or smell, sore throat, congestion, runny nose, nausea, vomiting, and diarrhea. In some embodiments, a single dose of the pharmaceutical composition contains 10 nanograms to 1 milligram, 0.1-250 micrograms, 10-100 micrograms, or 12.5-50 micrograms of the recombinant polypeptide. In some embodiments, a single dose of the pharmaceutical composition contains 0.01-1, 0.1-1, 0.5-5, 1-20, or 5-15, 1-50, or 10-50 micrograms of the recombinant polypeptide. In some embodiments, wherein the pharmaceutical composition does not contain an adjuvant, a single dose of the pharmaceutical composition could contain. 01-1, 0.1-1, 0.5-5, 1-20, or 5-15, 1-50, or 10-50 micrograms of the recombinant polypeptide. However, a single dose of the pharmaceutical composition could also contain increased amounts of the recombinant polypeptide, such as 100-1000, 100-250, or 250-500 micrograms of recombinant polypeptide.

A further embodiment is a method for preventing, inhibiting, reducing, eliminating, protecting against, or delaying the onset of an infection or an infectious clinical condition caused by a beta coronavirus in a subject comprising administering to the subject the recombinant polypeptide described herein, the polypeptide encoded by the recombinant polynucleotide described herein, or a dose of the pharmaceutical composition described herein. Examples of beta coronaviruses include SARS-COV, MERS-COV, and SARS-COV-2.

Another embodiment is a method for inducing an immune response against a beta coronavirus in a subject comprising administering to the subject the recombinant polypeptide described herein, the polypeptide encoded by the recombinant polynucleotide described herein, or a dose of the pharmaceutical composition described herein. Examples of beta coronaviruses include SARS-COV, MERS-COV, and SARS-COV-2.

In the methods described herein, administration can be achieved by any suitable administration method. Suitable administration methods include oral, parenteral, subcutaneous, intravenous, intramuscular, intrapulmonary, intranasal, intraarterial, intrathecal, and intraperitoneal administration.

In the methods described herein, the subject can be any suitable animal that is capable of being infected by a beta coronavirus, such as SARS-COV, MERS-COV, and SARS-COV-2. The subject can be a human, non-human primate, horse, pig, cattle, cat, dog, sheep, mink, rodent, hamster, or bat, preferably human.

### Examples of Non-Limiting Aspects of the Disclosure

Aspects, including embodiments, of the invention described herein may be beneficial alone or in combination, with one or more other aspects or embodiments. Without limiting the foregoing description, certain non-limiting aspects of the disclosure numbered (1)-(36) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A recombinant polypeptide comprising at least one immunogenic fragment of Severe Acute Respiratory Syndrome Coronavirus 2 (SARS-COV-2) spike glycoprotein and an antibody Fc region.

(2) The recombinant polypeptide of aspect 1, wherein the at least one fragment of the SARS-COV-2 spike glycoprotein comprises the N-terminal domain of the S1 subunit, the C-terminal domain of the S1 subunit, or both.

(3) The recombinant polypeptide of aspect 1 or 2, wherein the at least one fragment of the SARS-COV-2 spike glycoprotein comprises an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 245 (CTD_short_h) 2), SEQ ID NO: 249 (LS2435 Single domain_(GAMMA-Plus)), SEQ ID NO: 253 (BETA_CTD_short_i), SEQ ID NO: 257 (GAMMA_CTD_short_i), SEQ ID NO: 261 (GAMMA_CTD_short_i), SEQ ID NO: 265 (DELTA-Plus_CTD_short_i), SEQ ID NO: 269 (MU_CTD_short_i), SEQ ID NO: 273 (A.30_CTD_short_i), SEQ ID NO: 277 (DELTA-Plus+MU_CTD_short_i), SEQ ID NO: 281 (DELTA-Plus+A.30_CTD_short_i), SEQ ID NO: 285 (BETA_CTD_short_i)₂), SEQ ID NO: 289 ((GAMMA_CTD_short_i)₂), SEQ ID NO: 293 ((DELTA_CTD_short_i)₂), SEQ ID NO: 297 ((DELTA-Plus_CTD_short_i)₂), SEQ ID NO: 301 ((MU_CTD_short_i)₂), SEQ ID NO: 305 ((A.30_CTD_short_i)₂), SEQ ID NO: 309 ((DELTA-Plus+MU_CTD_short_i)₂), SEQ ID NO: 313 ((DELTA-Plus+A.30_CTD_short_i)₂), SEQ ID NO: 317 (Omicron_Boost_CTD_short_i), SEQ ID NO: 321 ((Omicron_Boost_CTD_short_i)₂), SEQ ID NO: 325 (Omicron_CTD_short_i), SEQ ID NO: 329 ((Omicron_CTD_short_i)₂), SEQ ID NO: 333 (Δ+μ_1_CTD_short_i), SEQ ID NO: 337 ((Δ+μ_2_CTD_short_i)₂), SEQ ID NO: 341 (OΔ+μ_2_CTD_short_i), SEQ ID NO: 345 ((OΔ+μ_2_CTD_short_i)₂), SEQ ID NO: 349 (OΔ+μ_3_CTD_short_i), SEQ ID NO: 353 ((OΔ+μ_3_CTD_short_i)₂), SEQ ID NO: 357 (OΔ+μ_4_CTD_short_i), SEQ ID NO: 361 ((OΔ+μ_4_CTD_short_i)₂), SEQ ID NO: 365 (CTD-gCd01), SEQ ID NO: 366 (CTD-gCd02), SEQ ID NO: 367 (CTD-gCd03), SEQ ID NO: 368 (CTD-gCd04), SEQ ID NO: 369 (CTD-gCd05), SEQ ID NO: 370 (CTD-gCd06), SEQ ID NO: 371 (CTD-gCd07), SEQ ID NO: 372 (CTD-gCd08), SEQ ID NO: 373 (CTD-gCd09), SEQ ID NO: 374 (CTD-gCd10), SEQ ID NO: 375 (CTD-gCd11), SEQ ID NO: 376 (CTD-gCd12), SEQ ID NO: 377 (CTD-gCd13), SEQ ID NO: 378 (CTD-gCd14), SEQ ID NO: 379 (CTD-gCd15), SEQ ID NO: 380 (CTD-gCd16), SEQ ID NO: 381 (CTD-gCd17), SEQ ID NO: 382 (CTD-gCd18), SEQ ID NO: 383 (CTD-gCd19), SEQ ID NO: 384 (CTD-gCd20), SEQ ID NO: 385 (CTD-gCd21), SEQ ID NO: 386 (CTD-gCd22), SEQ ID NO: 387 (CTD-gCd23), SEQ ID NO: 388 (CTD-gCd24), SEQ ID NO: 389 (CTD-gCd25), SEQ ID NO: 390 (CTD-gCd26), SEQ ID NO: 391 (CTD-gCd27), SEQ ID NO: 392 (CTD-gCd28), SEQ ID NO: 393 (CTD-gCd29), SEQ ID NO: 394 (CTD-gCd30), SEQ ID NO: 395 (CTD-gCd31), SEQ ID NO: 396 (CTD-gCd32), SEQ ID NO: 397 (CTD-gCd33), SEQ ID NO: 398 (CTD-gCd34), SEQ ID NO: 399 (CTD-gCd35), SEQ ID NO: 400 (CTD-gCd36), SEQ ID NO: 401 (CTD-gCd37), SEQ ID NO: 402 (CTD-gCd38), SEQ ID NO: 403 (CTD-gN01), SEQ ID NO: 404 (CTD-gN02), SEQ ID NO: 405 (CTD-gN03), SEQ ID NO: 406 (CTD-gN04), SEQ ID NO: 407 (CTD-gN05), SEQ ID NO: 408 (CTD-gN06), SEQ ID NO: 409 (CTD-gN07), SEQ ID NO: 410 (CTD-gN08), SEQ ID NO: 411 (CTD-gN09), SEQ ID NO: 412 (CTD-gN10), SEQ ID NO: 413 (CTD-gN11), SEQ ID NO: 414 (CTD-gN12), SEQ ID NO: 415 (CTD-gN13) SEQ ID NO: 416 (CTD-gN14), SEQ ID NO: 417 (CTD-gN15), SEQ ID NO: 418 (CTD-gN16), SEQ ID NO: 419 (CTD-gN17), SEQ ID NO: 420 (CTD-gN18), SEQ ID NO: 421 (CTD-gN19), SEQ ID NO: 422 (CTD-gN20), SEQ ID NO: 423 (CTD-gN21), SEQ ID NO: 424 (CTD-gN22), SEQ ID NO: 425 (CTD-gN23), SEQ ID NO: 426 (CTD-gN24), SEQ ID NO: 427 (CTD-gN25), SEQ ID NO: 428 (CTD-gN26), SEQ ID NO: 429 (CTD-gN27), SEQ ID NO: 430 (CTD-gN28), SEQ ID NO: 431 (CTD-gN29), SEQ ID NO: 432 (CTD-gN30), SEQ ID NO: 433 (CTD-gN31), SEQ ID NO: 434 (CTD-gN32), SEQ ID NO: 435 (CTD-gN33), SEQ ID NO: 436 (CTD-gN34), SEQ ID NO: 437 (CTD-gN35), SEQ ID NO: 438 (CTD-gN36), SEQ ID NO: 439 (CTD-gN37), SEQ ID NO: 440 (CTD-gN38), SEQ ID NO: 441 (CTD-gN39), SEQ ID NO: 442 (CTD-gN40), SEQ ID NO: 443 (CTD-gN41), SEQ ID NO: 444 (CTD-gN42), SEQ ID NO: 445 (CTD-gN43), SEQ ID NO: 446 (CTD-gN44), SEQ ID NO: 447 (CTD-gN45), SEQ ID NO: 448 (CTD-gN46), SEQ ID NO: 449 (CTD-gN47), and SEQ ID NOs: 705-716.

(4) The recombinant polypeptide of any one of aspects 1-3, wherein the polypeptide comprises an amino acid sequence selected from the group consisting of SEQ ID NOS: 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365-449, and 705-716.

(5) The recombinant polypeptide of any of one aspects 1-4, wherein the polypeptide comprises at least two immunogenic fragments.

(6) The recombinant polypeptide of aspect 5, wherein each of the at least two immunogenic fragments comprises an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 245 (CTD_short_h) 2), SEQ ID NO: 249 (LS2435 Single domain_(GAMMA-Plus)), SEQ ID NO: 253 (BETA_CTD_short_i), SEQ ID NO: 257 (GAMMA_CTD_short_i), SEQ ID NO: 261 (GAMMA_CTD_short_i), SEQ ID NO: 265 (DELTA-Plus_CTD_short_i), SEQ ID NO: 269 (MU_CTD_short_i), SEQ ID NO: 273 (A.30_CTD_short_i), SEQ ID NO: 277 (DELTA-Plus+MU_CTD_short_i), SEQ ID NO: 281 (DELTA-Plus+A.30_CTD_short_i), SEQ ID NO: 285 (BETA_CTD_short_i)₂), SEQ ID NO: 289 ((GAMMA_CTD_short_i)₂), SEQ ID NO: 293 ((DELTA_CTD_short_i)₂), SEQ ID NO: 297 ((DELTA-Plus_CTD_short_i)₂), SEQ ID NO: 301 ((MU_CTD_short_i)₂), SEQ ID NO: 305 ((A.30_CTD_short_i)₂), SEQ ID NO: 309 ((DELTA-Plus+MU_CTD_short_i)₂), SEQ ID NO: 313 ((DELTA-Plus+A.30_CTD_short_i)₂), SEQ ID NO: 317 (Omicron_Boost_CTD_short_i), SEQ ID NO: 321 ((Omicron_Boost_CTD_short_i)₂), SEQ ID NO: 325 (Omicron_CTD_short_i), SEQ ID NO: 329 ((Omicron_CTD_short_i)₂), SEQ ID NO: 333 (OΔ+μ_1_CTD_short_i), SEQ ID NO: 337 ((OΔ+μ_1_CTD_short_i)₂), SEQ ID NO: 341 (OΔ+μ_2_CTD_short_i), SEQ ID NO: 345 ((OΔ+μ_2_CTD_short_i)₂), SEQ ID NO: 349 (OΔ+μ_3_CTD_short_i), SEQ ID NO: 353 ((Δ+μ_3_CTD_short_i)₂), SEQ ID NO: 357 (Δ+μ_4_CTD_short_i), SEQ ID NO: 361 ((OΔ+μ_4_CTD_short_i)₂), SEQ ID NO: 365 (CTD-gCd01), SEQ ID NO: 366 (CTD-gCd02), SEQ ID NO: 367 (CTD-gCd03), SEQ ID NO: 368 (CTD-gCd04), SEQ ID NO: 369 (CTD-gCd05), SEQ ID NO: 370 (CTD-gCd06), SEQ ID NO: 371 (CTD-gCd07), SEQ ID NO: 372 (CTD-gCd08), SEQ ID NO: 373 (CTD-gCd09), SEQ ID NO: 374 (CTD-gCd10), SEQ ID NO: 375 (CTD-gCd11), SEQ ID NO: 376 (CTD-gCd12), SEQ ID NO: 377 (CTD-gCd13), SEQ ID NO: 378 (CTD-gCd14), SEQ ID NO: 379 (CTD-gCd15), SEQ ID NO: 380 (CTD-gCd16), SEQ ID NO: 381 (CTD-gCd17), SEQ ID NO: 382 (CTD-gCd18), SEQ ID NO: 383 (CTD-gCd19), SEQ ID NO: 384 (CTD-gCd20), SEQ ID NO: 385 (CTD-gCd21), SEQ ID NO: 386 (CTD-gCd22), SEQ ID NO: 387 (CTD-gCd23), SEQ ID NO: 388 (CTD-gCd24), SEQ ID NO: 389 (CTD-gCd25), SEQ ID NO: 390 (CTD-gCd26), SEQ ID NO: 391 (CTD-gCd27), SEQ ID NO: 392 (CTD-gCd28), SEQ ID NO: 393 (CTD-gCd29), SEQ ID NO: 394 (CTD-gCd30), SEQ ID NO: 395 (CTD-gCd31), SEQ ID NO: 396 (CTD-gCd32), SEQ ID NO: 397 (CTD-gCd33), SEQ ID NO: 398 (CTD-gCd34), SEQ ID NO: 399 (CTD-gCd35), SEQ ID NO: 400 (CTD-gCd36), SEQ ID NO: 401 (CTD-gCd37), SEQ ID NO: 402 (CTD-gCd38), SEQ ID NO: 403 (CTD-gN01), SEQ ID NO: 404 (CTD-gN02), SEQ ID NO: 405 (CTD-gN03), SEQ ID NO: 406 (CTD-gN04), SEQ ID NO: 407 (CTD-gN05), SEQ ID NO: 408 (CTD-gN06), SEQ ID NO: 409 (CTD-gN07), SEQ ID NO: 410 (CTD-gN08), SEQ ID NO: 411 (CTD-gN09), SEQ ID NO: 412 (CTD-gN10), SEQ ID NO: 413 (CTD-gN11), SEQ ID NO: 414 (CTD-gN12), SEQ ID NO: 415 (CTD-gN13) SEQ ID NO: 416 (CTD-gN14), SEQ ID NO: 417 (CTD-gN15), SEQ ID NO: 418 (CTD-gN16), SEQ ID NO: 419 (CTD-gN17), SEQ ID NO: 420 (CTD-gN18), SEQ ID NO: 421 (CTD-gN19), SEQ ID NO: 422 (CTD-gN20), SEQ ID NO: 423 (CTD-gN21), SEQ ID NO: 424 (CTD-gN22), SEQ ID NO: 425 (CTD-gN23), SEQ ID NO: 426 (CTD-gN24), SEQ ID NO: 427 (CTD-gN25), SEQ ID NO: 428 (CTD-gN26), SEQ ID NO: 429 (CTD-gN27), SEQ ID NO: 430 (CTD-gN28), SEQ ID NO: 431 (CTD-gN29), SEQ ID NO: 432 (CTD-gN30), SEQ ID NO: 433 (CTD-gN31), SEQ ID NO: 434 (CTD-gN32), SEQ ID NO: 435 (CTD-gN33), SEQ ID NO: 436 (CTD-gN34), SEQ ID NO: 437 (CTD-gN35), SEQ ID NO: 438 (CTD-gN36), SEQ ID NO: 439 (CTD-gN37), SEQ ID NO: 440 (CTD-gN38), SEQ ID NO: 441 (CTD-gN39), SEQ ID NO: 442 (CTD-gN40), SEQ ID NO: 443 (CTD-gN41), SEQ ID NO: 444 (CTD-gN42), SEQ ID NO: 445 (CTD-gN43), SEQ ID NO: 446 (CTD-gN44), SEQ ID NO: 447 (CTD-gN45), SEQ ID NO: 448 (CTD-gN46), SEQ ID NO: 449 (CTD-gN47), and SEQ ID NOs: 705-716.

(7) The recombinant polypeptide of aspect 5, wherein each of the at least two immunogenic fragments comprises an amino acid sequence selected from the group consisting of SEQ ID NOS: 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365-449, and 705-716.

(8) The recombinant polypeptide of any one of aspects 5-7, wherein each immunogenic fragment of the at least two immunogenic fragments comprises the same amino acid sequence.

(9) The recombinant polypeptide of any one of aspects 5-8, wherein each immunogenic fragment of the at least two immunogenic fragments comprises a different amino acid sequence from the other immunogenic fragments.

(10) The recombinant polypeptide of any one of aspects 1-9, wherein the polypeptide comprises two, three, four, or five immunogenic fragments.

(11) The recombinant polypeptide of any one of aspects 5-10, wherein the at least two immunogenic fragments are connected to each other via a linker.

(12) The recombinant polypeptide of aspect 11, wherein the linker is a polypeptide comprising an amino acid sequence of 1-35 residues, wherein each residue is independently serine or glycine.

(13) The recombinant polypeptide of any one of aspects 1-12, wherein the at least one immunogenic fragment of the SARS-COV-2 spike glycoprotein is connected to the antibody Fc region via a linker.

(14) The recombinant polypeptide of aspect 13, wherein the linker comprises an amino acid sequence selected from the group consisting of SEQ ID NO: 65 (Fc1), SEQ ID NO: 67 (Fc1-TEV), SEQ ID NO: 69 (Fc1-Rv3C), SEQ ID NO: 193 (Short), SEQ ID NO: 195 (Medium), and SEQ ID NO: 197 (Long).

(15) The recombinant polypeptide of any one of aspects 1-14, wherein the antibody Fc region is from a human IgG1 antibody or derived therefrom.

(16) The recombinant polypeptide of aspect 15, wherein the antibody Fc region comprises the amino acid sequence of SEQ ID NO: 71.

(17) The recombinant polypeptide of any one of aspects 1-16, wherein the polypeptide comprises an amino acid sequence with at least 90%, preferably at least 91%, 92%, 93%, 94%, 95%, 96%, 97%, 98%, or 99%, sequence identity to an amino acid sequence selected from the group consisting of SEQ ID NO: 247 (CTD_short_h) 2-Fc), SEQ ID NO: 251 (LS2435 Single domain_(GAMMA-Plus)-Fc), SEQ ID NO: 255 (BETA_CTD_short_i-Fc), SEQ ID NO: 259 (GAMMA_CTD_short_i-Fc), SEQ ID NO: 263 (GAMMA_CTD_short_i-Fc), SEQ ID NO: 267 (DELTA-Plus_CTD_short_i-Fc), SEQ ID NO: 271 (MU_CTD_short_i-Fc), SEQ ID NO: 275 (A.30_CTD_short_i-Fc), SEQ ID NO: 279 (DELTA-Plus+MU_CTD_short_i-Fc), SEQ ID NO: 283 (DELTA-Plus+A.30_CTD_short_i-Fc), SEQ ID NO: 287 (BETA_CTD_short_i)₂-Fc), SEQ ID NO: 291 ((GAMMA_CTD_short_i)₂-Fc), SEQ ID NO: 295 ((DELTA_CTD_short_i)₂-Fc), SEQ ID NO: 299 ((DELTA-Plus_CTD_short_i)₂-Fc), SEQ ID NO: 303 ((MU_CTD_short_i)₂-Fc), SEQ ID NO: 307 ((A.30_CTD_short_i)₂-Fc), SEQ ID NO: 311 ((DELTA-Plus+MU_CTD_short_i)₂-Fc), SEQ ID NO: 315 ((DELTA-Plus+A.30_CTD_short_i)₂-Fc), SEQ ID NO: 319 (Omicron_Boost_CTD_short_i-Fc), SEQ ID NO: 323 ((Omicron_Boost_CTD_short_i)₂-Fc), SEQ ID NO: 327 (Omicron_CTD_short_i-Fc), SEQ ID NO: 331 ((Omicron_CTD_short_i)₂-Fc), SEQ ID NO: 335 (OΔ+μ_1_CTD_short_i-Fc), SEQ ID NO: 339 ((OΔ+μ_1_CTD_short_i)₂-Fc), SEQ ID NO: 343 (OΔ+μ_2_CTD_short_i-Fc), SEQ ID NO: 347 ((OΔ+μ_2_CTD_short_i)₂-Fc), SEQ ID NO: 351 (OΔ+μ_3_CTD_short_i-Fc), SEQ ID NO: 355 ((OΔ+μ_3_CTD_short_i)₂-Fc), SEQ ID NO: 359 (OΔ+μ_4_CTD_short_i-Fc), SEQ ID NO: 363 ((OΔ+μ_4_CTD_short_i)₂-Fc),), SEQ ID NO: 535 (CTD-gCd01-Fc), SEQ ID NO: 536 (CTD-gCd02-Fc), SEQ ID NO: 537 (CTD-gCd03-Fc), SEQ ID NO: 538 (CTD-gCd04-Fc), SEQ ID NO: 539 (CTD-gCd05-Fc), SEQ ID NO: 540 (CTD-gCd06-Fc), SEQ ID NO: 541 (CTD-gCd07-Fc), SEQ ID NO: 542 (CTD-gCd08-Fc), SEQ ID NO: 543 (CTD-gCd09-Fc), SEQ ID NO: 544 (CTD-gCd10-Fc), SEQ ID NO: 545 (CTD-gCd11-Fc), SEQ ID NO: 546 (CTD-gCd12-Fc), SEQ ID NO: 547 (CTD-gCd13-Fc), SEQ ID NO: 548 (CTD-gCd14-Fc), SEQ ID NO: 549 (CTD-gCd15-Fc), SEQ ID NO: 550 (CTD-gCd16-Fc), SEQ ID NO: 551 (CTD-gCd17-Fc), SEQ ID NO: 552 (CTD-gCd18-Fc), SEQ ID NO: 553 (CTD-gCd19-Fc), SEQ ID NO: 554 (CTD-gCd20-Fc), SEQ ID NO: 555 (CTD-gCd21-Fc), SEQ ID NO: 556 (CTD-gCd22-Fc), SEQ ID NO: 557 (CTD-gCd23-Fc), SEQ ID NO: 558 (CTD-gCd24-Fc), SEQ ID NO: 559 (CTD-gCd25-Fc), SEQ ID NO: 560 (CTD-gCd26-Fc), SEQ ID NO: 561 (CTD-gCd27-Fc), SEQ ID NO: 562 (CTD-gCd28-Fc), SEQ ID NO: 563 (CTD-gCd29-Fc), SEQ ID NO: 564 (CTD-gCd30-Fc), SEQ ID NO: 565 (CTD-gCd31-Fc), SEQ ID NO: 566 (CTD-gCd32-Fc), SEQ ID NO: 567 (CTD-gCd33-Fc), SEQ ID NO: 568 (CTD-gCd34-Fc), SEQ ID NO: 569 (CTD-gCd35-Fc), SEQ ID NO: 570 (CTD-gCd36-Fc), SEQ ID NO: 571 (CTD-gCd37-Fc), SEQ ID NO: 572 (CTD-gCd38-Fc), SEQ ID NO: 573 (CTD-gN01-Fc), SEQ ID NO: 574 (CTD-gN02), SEQ ID NO: 575 (CTD-gN03-Fc), SEQ ID NO: 576 (CTD-gN04-Fc), SEQ ID NO: 577 (CTD-gN05-Fc), SEQ ID NO: 578 (CTD-gN06-Fc), SEQ ID NO: 579 (CTD-gN07-Fc), SEQ ID NO: 580 (CTD-gN08-Fc), SEQ ID NO: 581 (CTD-gN09-Fc), SEQ ID NO: 582 (CTD-gN10-Fc), SEQ ID NO: 583 (CTD-gN11-Fc), SEQ ID NO: 584 (CTD-gN12-Fc), SEQ ID NO: 585 (CTD-gN13-Fc) SEQ ID NO: 586 (CTD-gN14-Fc), SEQ ID NO: 587 (CTD-gN15-Fc), SEQ ID NO: 588 (CTD-gN16-Fc), SEQ ID NO: 589 (CTD-gN17-Fc), SEQ ID NO: 590 (CTD-gN18-Fc), SEQ ID NO: 591 (CTD-gN19-Fc), SEQ ID NO: 592 (CTD-gN20-Fc), SEQ ID NO: 593 (CTD-gN21-Fc), SEQ ID NO: 594 (CTD-gN22-Fc), SEQ ID NO: 595 (CTD-gN23-Fc), SEQ ID NO: 596 (CTD-gN24-Fc), SEQ ID NO: 597 (CTD-gN25-Fc), SEQ ID NO: 598 (CTD-gN26-Fc), SEQ ID NO: 599 (CTD-gN27-Fc), SEQ ID NO: 600 (CTD-gN28-Fc), SEQ ID NO: 601 (CTD-gN29-Fc), SEQ ID NO: 602 (CTD-gN30-Fc), SEQ ID NO: 603 (CTD-gN31-Fc), SEQ ID NO: 604 (CTD-gN32-Fc), SEQ ID NO: 605 (CTD-gN33-Fc), SEQ ID NO: 606 (CTD-gN34-Fc), SEQ ID NO: 607 (CTD-gN35-Fc), SEQ ID NO: 608 (CTD-gN36-Fc), SEQ ID NO: 609 (CTD-gN37-Fc), SEQ ID NO: 610 (CTD-gN38-Fc), SEQ ID NO: 611 (CTD-gN39-Fc), SEQ ID NO: 612 (CTD-gN40-Fc), SEQ ID NO: 613 (CTD-gN41-Fc), SEQ ID NO: 614 (CTD-gN42-Fc), SEQ ID NO: 615 (CTD-gN43-Fc), SEQ ID NO: 616 (CTD-gN44-Fc), SEQ ID NO: 617 (CTD-gN45-Fc), SEQ ID NO: 618 (CTD-gN46-Fc), SEQ ID NO: 619 (CTD-gN47), and SEQ ID NOs: 705-716.

(18) The recombinant polypeptide of aspect 17, wherein the polypeptide comprises an amino acid sequence selected from the group consisting of SEQ ID NOS: 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 535-619, and SEQ ID NOs: 705-716.

(19) A recombinant polynucleotide encoding the recombinant polypeptide of any one of aspects 1-18.

(20) The recombinant polynucleotide of aspect 19, wherein the polynucleotide comprises a nucleic acid sequence with at least 90%, preferably at least 91%, 92%, 93%, 94%, 95%, 96%, 97%, 98%, or 99%, sequence identity to a nucleic acid sequence selected from the group consisting of SEQ ID NO: 248 (CTD_short_h) 2-Fc), SEQ ID NO: 252 (LS2435 Single domain_(GAMMA-Plus)-Fc), SEQ ID NO: 256 (BETA_CTD_short_i-Fc), SEQ ID NO: 260 (GAMMA_CTD_short_i-Fc), SEQ ID NO: 264 (GAMMA_CTD_short_i-Fc), SEQ ID NO: 268 (DELTA-Plus_CTD_short_i-Fc), SEQ ID NO: 272 (MU_CTD_short_i-Fc), SEQ ID NO: 276 (A.30_CTD_short_i-Fc), SEQ ID NO: 280 (DELTA-Plus+MU_CTD_short_i-Fc), SEQ ID NO: 284 (DELTA-Plus+A.30_CTD_short_i-Fc), SEQ ID NO: 288 (BETA_CTD_short_i)₂-Fc), SEQ ID NO: 292 ((GAMMA_CTD_short_i)₂-Fc), SEQ ID NO: 296 ((DELTA_CTD_short_i)₂-Fc), SEQ ID NO: 300 ((DELTA-Plus_CTD_short_i)₂-Fc), SEQ ID NO: 304 ((MU_CTD_short_i)₂-Fc), SEQ ID NO: 308 ((A.30_CTD_short_i)₂-Fc), SEQ ID NO: 312 ((DELTA-Plus+MU_CTD_short_i)₂-Fc), SEQ ID NO: 316 ((DELTA-Plus+A.30_CTD_short_i)₂-Fc), SEQ ID NO: 320 (Omicron_Boost_CTD_short_i-Fc), SEQ ID NO: 324 ((Omicron_Boost_CTD short_i)₂-Fc), SEQ ID NO: 328 (Omicron_CTD_short_i-Fc), SEQ ID NO: 332 ((Omicron_CTD short_i)₂-Fc), SEQ ID NO: 336 (OΔ+μ_1_CTD_short_i-Fc), SEQ ID NO: 340 ((Δ+μ-1_CTD_short_i)₂-Fc), SEQ ID NO: 344 (Δ+μ_2_CTD_short_i-Fc), SEQ ID NO: 348 ((Δ+μ-2_CTD_short_i)₂-Fc), SEQ ID NO: 352 (Δ+μ_3_CTD_short_i-Fc), SEQ ID NO: 356 ((Δ+μ_3_CTD_short_i)₂-Fc), SEQ ID NO: 360 (Δ+μ_4_CTD_short_i-Fc), SEQ ID NO: 364 ((OΔ+μ_4_CTD_short_i)₂-Fc), SEQ ID NO: 620 (CTD-gCd01-Fc), SEQ ID NO: 621 (CTD-gCd02-Fc), SEQ ID NO: 622 (CTD-gCd03-Fc), SEQ ID NO: 623 (CTD-gCd04-Fc), SEQ ID NO: 624 (CTD-gCd05-Fc), SEQ ID NO: 625 (CTD-gCd06-Fc), SEQ ID NO: 626 (CTD-gCd07-Fc), SEQ ID NO: 627 (CTD-gCd08-Fc), SEQ ID NO: 628 (CTD-gCd09-Fc), SEQ ID NO: 629 (CTD-gCd10-Fc), SEQ ID NO: 630 (CTD-gCd11-Fc), SEQ ID NO: 631 (CTD-gCd12-Fc), SEQ ID NO: 632 (CTD-gCd13-Fc), SEQ ID NO: 633 (CTD-gCd14-Fc), SEQ ID NO: 634 (CTD-gCd15-Fc), SEQ ID NO: 635 (CTD-gCd16-Fc), SEQ ID NO: 636 (CTD-gCd17-Fc), SEQ ID NO: 637 (CTD-gCd18-Fc), SEQ ID NO: 638 (CTD-gCd19-Fc), SEQ ID NO: 639 (CTD-gCd20-Fc), SEQ ID NO: 640 (CTD-gCd21-Fc), SEQ ID NO: 641 (CTD-gCd22-Fc), SEQ ID NO: 642 (CTD-gCd23-Fc), SEQ ID NO: 643 (CTD-gCd24-Fc), SEQ ID NO: 644 (CTD-gCd25-Fc), SEQ ID NO: 645 (CTD-gCd26-Fc), SEQ ID NO: 646 (CTD-gCd27-Fc), SEQ ID NO: 647 (CTD-gCd28-Fc), SEQ ID NO: 648 (CTD-gCd29-Fc), SEQ ID NO: 649 (CTD-gCd30-Fc), SEQ ID NO: 650 (CTD-gCd31-Fc), SEQ ID NO: 651 (CTD-gCd32-Fc), SEQ ID NO: 652 (CTD-gCd33-Fc), SEQ ID NO: 653 (CTD-gCd34-Fc), SEQ ID NO: 654 (CTD-gCd35-Fc), SEQ ID NO: 655 (CTD-gCd36-Fc), SEQ ID NO: 656 (CTD-gCd37-Fc), SEQ ID NO: 657 (CTD-gCd38-Fc), SEQ ID NO: 658 (CTD-gN01-Fc), SEQ ID NO: 659 (CTD-gN02), SEQ ID NO: 660 (CTD-gN03-Fc), SEQ ID NO: 661 (CTD-gN04-Fc), SEQ ID NO: 662 (CTD-gN05-Fc), SEQ ID NO: 663 (CTD-gN06-Fc), SEQ ID NO: 664 (CTD-gN07-Fc), SEQ ID NO: 665 (CTD-gN08-Fc), SEQ ID NO: 666 (CTD-gN09-Fc), SEQ ID NO: 667 (CTD-gN10-Fc), SEQ ID NO: 668 (CTD-gN11-Fc), SEQ ID NO: 669 (CTD-gN12-Fc), SEQ ID NO: 670 (CTD-gN13-Fc) SEQ ID NO: 671 (CTD-gN14-Fc), SEQ ID NO: 672 (CTD-gN15-Fc), SEQ ID NO: 673 (CTD-gN16-Fc), SEQ ID NO: 674 (CTD-gN17-Fc), SEQ ID NO: 675 (CTD-gN18-Fc), SEQ ID NO: 676 (CTD-gN19-Fc), SEQ ID NO: 677 (CTD-gN20-Fc), SEQ ID NO: 678 (CTD-gN21-Fc), SEQ ID NO: 679 (CTD-gN22-Fc), SEQ ID NO: 680 (CTD-gN23-Fc), SEQ ID NO: 681 (CTD-gN24-Fc), SEQ ID NO: 682 (CTD-gN25-Fc), SEQ ID NO: 683 (CTD-gN26-Fc), SEQ ID NO: 684 (CTD-gN27-Fc), SEQ ID NO: 685 (CTD-gN28-Fc), SEQ ID NO: 686 (CTD-gN29-Fc), SEQ ID NO: 687 (CTD-gN30-Fc), SEQ ID NO: 688 (CTD-gN31-Fc), SEQ ID NO: 689 (CTD-gN32-Fc), SEQ ID NO: 690 (CTD-gN33-Fc), SEQ ID NO: 691 (CTD-gN34-Fc), SEQ ID NO: 692 (CTD-gN35-Fc), SEQ ID NO: 693 (CTD-gN36-Fc), SEQ ID NO: 694 (CTD-gN37-Fc), SEQ ID NO: 695 (CTD-gN38-Fc), SEQ ID NO: 696 (CTD-gN39-Fc), SEQ ID NO: 697 (CTD-gN40-Fc), SEQ ID NO: 698 (CTD-gN41-Fc), SEQ ID NO: 699 (CTD-gN42-Fc), SEQ ID NO: 700 (CTD-gN43-Fc), SEQ ID NO: 701 (CTD-gN44-Fc), SEQ ID NO: 702 (CTD-gN45-Fc), SEQ ID NO: 703 (CTD-gN46-Fc), SEQ ID NO: 704 (CTD-gN47), and SEQ ID NOs: 717-728.

(21) The recombinant polynucleotide of aspect 20, wherein the polynucleotide comprises a nucleic acid sequence selected from the group consisting of SEQ ID NO: 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 620-704, and 717-728.

(22) The recombinant polynucleotide of any one of aspects 19-21, wherein the nucleic acid sequence has been codon optimized.

(23) A pharmaceutical composition comprising the recombinant polypeptide of any one of aspects 1-18 or the polypeptide encoded by the recombinant polynucleotide of any one of aspects 19-22, and a pharmaceutically acceptable carrier.

(24) The pharmaceutical composition of aspect 23, wherein the pharmaceutical composition further comprises at least one adjuvant.

(25) The pharmaceutical composition of aspect 24, wherein the at least one adjuvant is selected from the group consisting of alum adjuvants, emulsion adjuvants, and pattern recognition receptor agonist adjuvants.

(26) The pharmaceutical composition of aspect 25, wherein the at least one adjuvant is MF59, Squalene Emulsion, Alum, aluminum hydroxide gels, calcium phosphate hydroxide, paraffin oil, cytokines (IL-1, IL-2, IL-12), killed bacterial products such as Bordetella and Mycobacterium bacteria, bacterial toxoids, squalene and DL-α-tocopherol emulsions, aluminum phosphate gels, saponins, cyclic dinucleotides, and TLR agonists, preferably TLR1, TLR2, TLR4, TLR5, TLR7, TLR8, TLR9 etc., and combinations thereof.

(27) The pharmaceutical composition of aspect 24, wherein the at least one adjuvant is a squalene-oil-in-water emulsion adjuvant.

(28) A vector comprising the recombinant polynucleotide of any one of aspects 19-22.

(29) An isolated cell comprising the recombinant polypeptide of any one of aspects 1-18, or the polypeptide encoded by the recombinant polynucleotide of any one of aspects 19-22.

(30) A method for preventing, inhibiting, reducing, eliminating, protecting, or delaying the onset of an infection or an infectious clinical condition caused by a beta coronavirus in a subject comprising administering to the subject the recombinant polypeptide of any one of aspects 1-18, the polypeptide encoded by the recombinant polynucleotide of any one of aspects 19-22, or the pharmaceutical composition of any one of aspects 23-27.

(31) A method for inducing an immune response against a beta coronavirus in a subject comprising administering to the subject the recombinant polypeptide of any one of aspects 1-18, the polypeptide encoded by the recombinant polynucleotide of any one of aspects 19-22, or the pharmaceutical composition of any one of aspects 23-27.

(32) The method of aspect 30 or 31, wherein the recombinant polypeptide of any one of aspects 1-18, the polypeptide encoded by the recombinant polynucleotide of any one of aspects 19-22, or the pharmaceutical composition of any one of aspects 23-27 is administered by oral, parenteral, subcutaneous, intravenous, intramuscular, intranasal, intrapulmonary, intraarterial, intrathecal, or interperitoneal administration.

(33) The method of any one of aspects 30-32, wherein the coronavirus is selected from the group consisting of SARS-COV-2, SARS-COV, and MERS-COV.

(34) The method of any one of aspects 30-33, wherein the subject is a mammal, preferably a human or non-human primate.

(35) The use of the recombinant polypeptide of any one of aspects 1-18, the polypeptide encoded by the recombinant polynucleotide of any one of aspects 19-22, or the pharmaceutical composition of any one of aspects 23-27 for the preparation of a medicament for the treatment or prevention of illness caused by SARS-COV-2.

(36) The recombinant polypeptide of any one of aspects 1-18, the polypeptide encoded by the recombinant polynucleotide of any one of aspects 19-22, or the pharmaceutical composition of any one of aspects 23-27 for use as a medicament.

### EXAMPLES

The following descriptions of cloning and protein expression apply to each example.

Cloning: Inserts encoding a certain fragment of SARS COV-2 virus Spike protein S1 were designed based prediction and previous Lytic Solutions expression data. Codon optimized cDNA was synthesized. The insert was cloned using either (a) restriction digests and DNA ligations or (b) NEB HiFi DNA assembly builder mix into a pcDNA3.1 vector containing appropriate secretion signal sequences, linkers, and tags for secreted fusion protein expression, as well as sequence encoding a human IgG1 Fc region. The resulting clone encoding a recombinant SARS COV-2 Spike protein fragment-Fc region fusion protein (“recombinant CoV-2 fusion protein”) was verified by either colony PCR and/or restriction digests. DNA sequencing was also used.

Protein Expression: Supercoiled plasmid of the verified clone was transiently transfected into CHO-S cells and expressed under the control of a constitutive promoter within cell culture conditions ranging from 30-37° C. and 3-10 days in CO₂ (8%) incubators with rotary shaking agitation at speeds of 150 RPM. Cells were removed by centrifugation and culture medium containing the recombinant CoV-2 fusion protein were passed over Protein A agarose to bind the Fc region-containing CoV-2 protein. Filtration could also have been used to remove cells. The column containing bound recombinant CoV-2 fusion protein was washed with phosphate buffered saline. The recombinant CoV-2 fusion protein bound to the Protein A column was eluted with low pH glycine followed by neutralization in pH8.0 Tris. The recombinant CoV-2 fusion protein was dialyzed to remove glycine/tris and placed into HEPES-buffered saline (10 mM HEPES. 150 mM NaCl, pH adjusted with NaOH to pH 7.2-7.5). No additional purification was employed in this case. However, additional purification by any chromatography method such as HIC or ion exchange can optionally be used.

### Example 1

SARS COV-2 antigens were selected from the genomic sequence (ncbi.nlm.nih.gov/nuccore/MN908947) to use in generating antibody and T-cell responses to the receptor binding domains of SARS COV-2 virus Spike protein. The selected domain encoding regions were codon optimized for CHO cell expression using IDTDNA codon optimization algorithms. Template DNA was synthesized at Twist Bio. A modified pcDNA3.1 vector was used for protein expression that included human IgG1 Fc for translational fusion generation, i.e., a fusion protein containing a SARS COV-2 virus Spike protein fragment and an antibody Fc region. The vector encoding the fusion protein was expressed in CHO cells, in which the fusion protein was secreted to the media, cells and cell debris were removed by centrifugation, the recombinant protein was captured with Protein A resin, and eluted with low pH glycine buffer. The resulting fusion protein was buffer exchanged by dialysis and mixed with adjuvant. The resulting vaccine composition was injected intramuscularly into Cynomolgus monkeys. The vaccine composition generated an unexpectedly strong immune response in Cynomolgus monkeys.

Vaccination: Following protein expression in accordance with the description above, the purified recombinant CoV-2 fusion protein (LS2330, CTD_short-a-Fc; SEQ ID NO: 73) was mixed with Titermax Gold adjuvant, a modified squalene in water emulsion adjuvant, according to manufacturer directions, and injected intramuscularly into the thigh of Cynomolgus monkeys. Injections were performed at day 0 and day 14. Dosages were 250 ug of antigen of CTD_short-a-Fc (Seq ID NO:73). Prior to the first injection on day 0, a baseline sample was collected from each test subject.

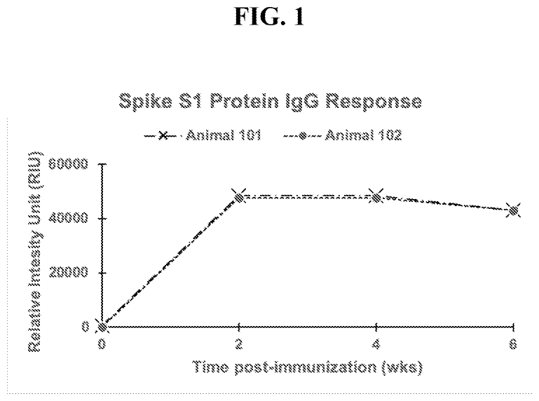

The immune response was monitored every two weeks following the injection on day 0, with the earliest sample taken on day 14. Accordingly, samples were collected on day 14 and day 28. The samples were analyzed, and the results are shown in FIG. 1. Venous blood was obtained from Cynomolgus monkeys pre immunization in EDTA-containing vacutubes (value shown as Day 0 serum sample in FIG. 1). Immediately after the pre-immunization blood sample was taken, monkeys were immunized with CTD_short-a-Fc (Seq ID NO:73). Serum was isolated by centrifugation of the non-coagulated blood. Serum was diluted 1:100 and analyzed on Intuitive Biosciences ELISA platform for anti-S1 spike binding antibodies. Additional time points were taken at 2 weeks post Day 0, 4 weeks post Day 0, and 6 weeks post Day 0. The single immune boost (CTD_short-a-Fc (Seq ID NO:73)) was performed immediately after the 2 week blood sample was taken. Samples were processed similar to Day 0 samples and immune response was recorded as Relative Intensity Units (RIU) on Intuitive Bioscience ELISA platform. Capture of anti-SARS COV-2 antibodies was performed by spotting SARS COV-2 S1 spike protein (Sino Biologicals) onto the wells of Intuitive Bioscience ELISA platform 96 well plates, adding diluted serum-full concentration serum and dilutions to 1:200 serum: buffer were used, but higher concentrations of serum surprisingly resulted in a signal too strong to read on the platform, thus requiring a 1:200 fold dilution for the readings shown in FIG. 1- and incubating for 10-120 minutes to allow binding of anti-CTD antibodies to the S1 spike protein attached to the plate wells. Serum was washed 3× form the wells, and followed by application of anti-cynomolgus detection antibody to the well, incubated for 10-120 minutes, then washed 3× from the well. Detection reagents were added and the signal was quantified compared to control spots of unrelated proteins.

FIG. 1 depicts an unexpectedly strong immune response in Cynomolgus monkeys to the tested construct, particularly in comparison to the immune response generated by other known SARS-COV-2 constructs. See, for instance, Graham et al., “Evaluation of the immunogenicity of prime-boost vaccination with the replication-deficient viral vectored COVID-19 vaccine candidate ChAOx1 nCOV-19,” bioRxiv preprint doi: doi.org/10.1101/2020.06.20.159715 (posted Jun. 20, 2020)

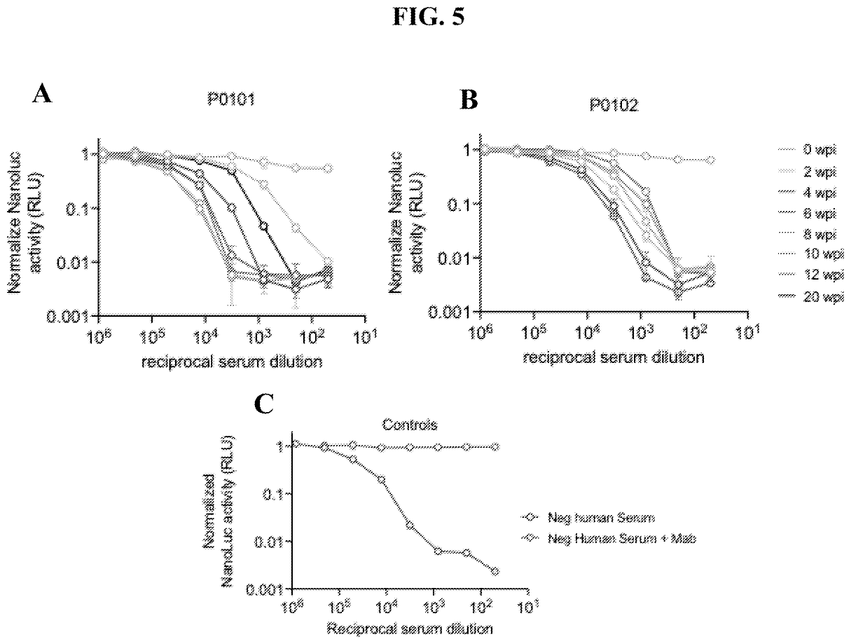

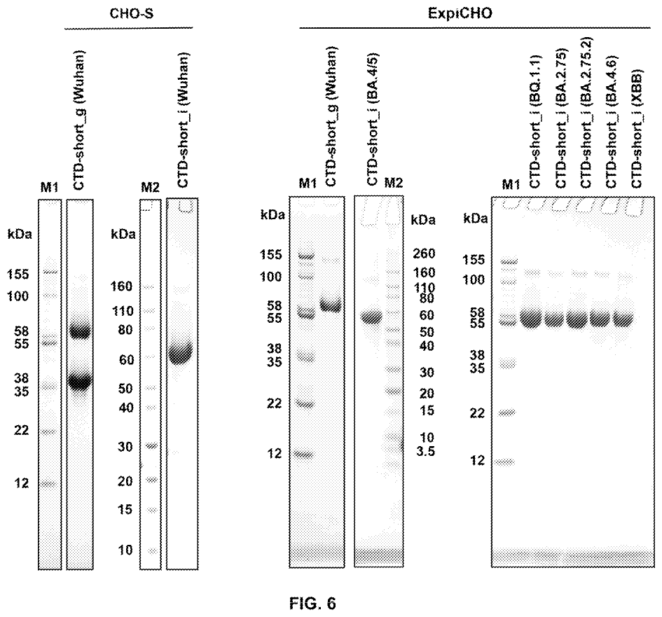

Neutralization Assays: FIGS. 5A-C depict the results of neutralization assays done on the tested Cynomolgus monkeys P0101 and P0102 (respectively depicted as “Animal 101” and “Animal 102” in FIG. 1). Regarding FIGS. 5A and 5B, “wpi” in the legend denotes weeks post injection/immunization. SARS-COV-2 pseudotyped particles were generated as previously described (see Schmidt, F., et al. Measuring SARS-COV-2 neutralizing antibody activity using pseudotyped and chimeric viruses. J Exp Med, v. 217, n. 11, 11 2020). Briefly, 293T cells were transfected with pHIV-1NLGagPol, pCCNG/nLuc and pSARS-COV-2-SΔ19. Particles were harvested 48 hours after transfection, filtered and stored at −80° C. Fourfold serially diluted serum from the immunized monkeys was incubated with SARS-COV-2 pseudotyped virus for 1 h at 37° C. The mixture was subsequently incubated with 293T/ACE2cl.22 cells (plated on Poly-D-Lysine-coated 96-well plates) with the final starting dilution of serum being 1:50. At 48 h later the cells were washed with PBS and lysed with Luciferase Cell Culture Lysis 5× reagent (Promega). Nanoluc Luciferase activity in lysates was measured using the Nano-Glo Luciferase Assay System (Promega) with the Modulus II Microplate Reader (Turner BioSystems). The raw nanoluc luciferase activity values (relative luminescence units) were normalized to those derived from cells infected with SARS-COV-2 pseudotyped virus in the absence of serum or a rabbit monoclonal antibody diluted in normal human serum at 0.105 mg/mL (40592-R001, Sinobiological, Wayne, PA). The half-maximal inhibitory concentration for serum (NT50) was determined using four-parameter nonlinear regression (GraphPad Prism).

Immunization of the two Cynomolgus macaques (IDs: P0101 and P0102) with SEQ ID NO: 73 (LS2330 [CTD_short_a-Fc] produced robust neutralizing antibody response. Neutralization assays were performed using a replication-defective single-cycle pseudotyped virus carrying SARS-COV-2 spikes and the NanoLuc luciferase reporter. This assay has been previously shown to accurately predict serum neutralizing activity against authentic SARS-COV-2 (see Schmidt, F., et al. referenced above). As a control for neutralization sensitivity, human serum obtained from a SARS-COV-2 negative individual was used alone or spiked with a monoclonal neutralizing antibody (FIG. 5C). Serum samples collected at the various timepoints from 0 to 20 weeks post-immunization were evaluated for neutralizing activity. Sera from animals immunized with SEQ ID NO: 73 (LS2330 [CTD_short_a-Fc] had readily detectable neutralization activity, as early as 2 weeks post-immunization that significantly increased until weeks 4 to 8 of the study. Indeed, neutralizing titers were exceptionally high at 4-8 weeks after immunization, in the range of 10,000 to 100,000 and were maintained in the 1000 to 10,000 range at 20 weeks after immunization.

### Example 2

Overview: SARS COV-2 antigens were selected from the SARS COV-2 genomic sequence (ncbi.nlm.nih.gov/nuccore/MN908947) to use in generating antibody and T-cell responses to the receptor binding domains of SARS COV-2 virus Spike protein. Preliminary data identified select individual regions of the SARS COV-2 virus Spike protein that would be amenable to high-level expression as Fc-fusions, that are resistant to proteolysis when expressed in CHO cells, and that generate strong immune response in Cynomolgus macaques. We further determined that in-series multimerization of select SARS COV-2 virus Spike protein domains can be used to create proteins that retain high-level expression without significant proteolysis sensitivity whilst doubling the theoretical antigenicity of the molecule to be used for immune stimulation. The design of the multiple domain molecules provides a scaffold for straight-forward modification to incorporate amino acid mutations identified in new and emerging variants of the SARS COV-2 virus Spike protein. The resulting vaccine composition of the Wuhan variant was injected intramuscularly into Rhesus macaques and elicit stronger immune responses using simple adjuvants at doses at mere fractions of that needed with single domain Fc-fusions.

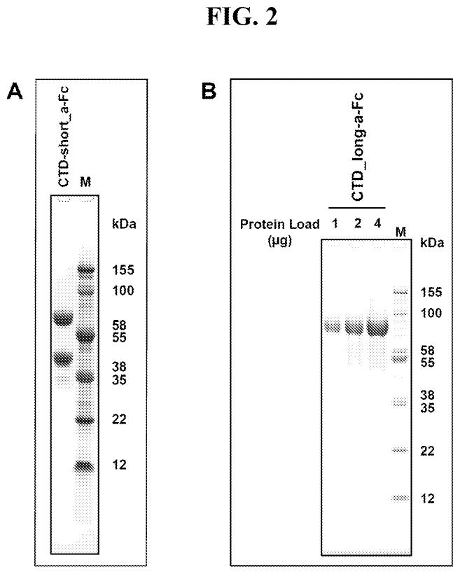

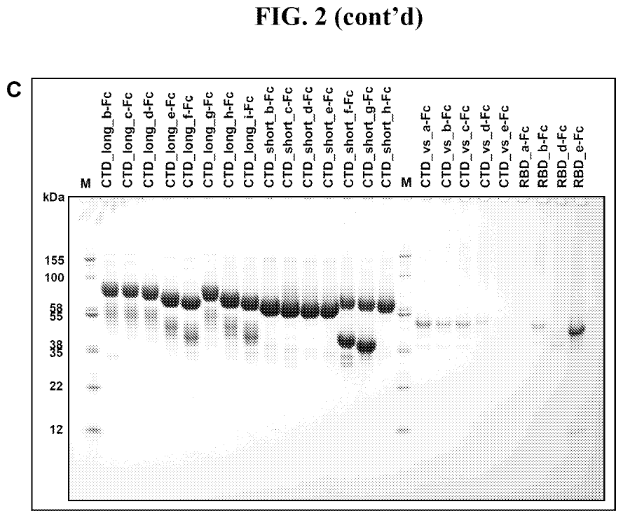

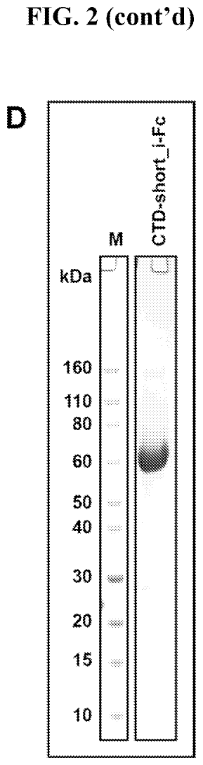

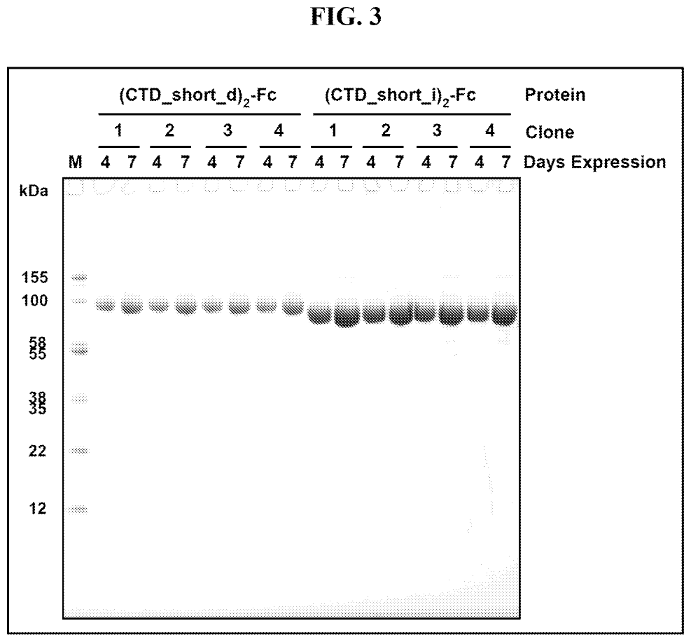

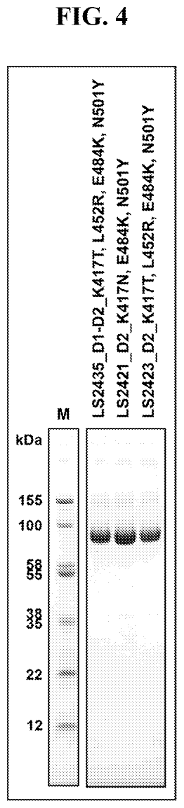

Following protein expression in accordance with the description above, resulting protein was analyzed by reducing SDS-PAGE to determine protein integrity for monomeric CTD-Fcs (FIGS. 2A-D), in-series dimer CTD-Fcs (FIG. 3) and in-series dimer mutant CTD-Fcs (FIG. 4). Protein yields were calculated using absorbance of 280 nm light and protein-specific extinction coefficients determined in silico according to their expected amino acid composition. Representative protein yields are given in Table 1.

TABLE 1

| | | Expression (mg |
|---|---|---|
| | | protein/L CHO |
| Strain(s) | Insert | culture (±SD)) |
| 3472 (SEQ ID NO: 75) | CTD_long_a (SEQ ID NO: 1) | 77.3 ⁽⁵⁾ |
| 2356 (SEQ ID NO: 101) | CTD_long_b (SEQ ID NO: 7) | 60.8 ⁽⁵⁾ |
| 2357 (SEQ ID NO: 103) | CTD_long_c (SEQ ID NO: 9) | 60.8 ⁽⁵⁾ |
| 2358 (SEQ ID NO: 105) | CTD_long_d (SEQ ID NO: 11) | 65.8 ⁽⁵⁾ |
| 2359 (SEQ ID NO: 107) | CTD_long_e (SEQ ID NO: 13) | 96.1 ⁽⁵⁾ |
| 2360 (SEQ ID NO: 109) | CTD_long_f (SEQ ID NO: 15) | 95.1 ⁽⁵⁾ |
| 2361 (SEQ ID NO: 111) | CTD_long_g (SEQ ID NO: 17) | 74.5 ⁽⁵⁾ |
| 2362 (SEQ ID NO: 113) | CTD_long_h (SEQ ID NO: 19) | 108.3 ⁽⁵⁾ |
| 2363 (SEQ ID NO: 115) | CTD_long_i (SEQ ID NO: 21) | 108.5 ⁽⁵⁾ |
| 2330 (SEQ ID NO: 73) | CTD_short_a (SEQ ID NO: 23) | >40 ⁽⁵⁾ |
| 2364 (SEQ ID NO: 117) | CTD_short_b (SEQ ID NO: 25) | 131.8 ⁽⁵⁾ |
| 2365 (SEQ ID NO: 119) | CTD_short_c (SEQ ID NO: 27) | 126.5 ⁽⁵⁾ |
| 2366 (SEQ ID NO: 121) | CTD_short_d (SEQ ID NO: 29) | 106.2 ⁽⁵⁾ |
| 2367 (SEQ ID NO: 123) | CTD_short_e (SEQ ID NO: 31) | 107.5 ⁽⁵⁾ |
| 2368 (SEQ ID NO: 125) | CTD_short_f (SEQ ID NO: 33) | 109.9 ⁽⁵⁾ |
| 2369 (SEQ ID NO: 127) | CTD_short_g (SEQ ID NO: 35) | 132.4 ⁽⁵⁾ |
| 2370 (SEQ ID NO: 129) | CTD_short_h (SEQ ID NO: 37) | 67.5 ⁽⁵⁾ |
| 2371 (SEQ ID NO: 131) | CTD_short_i (SEQ ID NO: 39) | 167.7 ⁽⁵⁾ |
| 2372 (SEQ ID NO: 133) | CTD_vs_a (SEQ ID NO: 41) | 2.3 ⁽⁵⁾ |
| 2373 (SEQ ID NO: 135) | CTD_vs_b (SEQ ID NO: 43) | 1.7 ⁽⁵⁾ |
| 2374 (SEQ ID NO: 137) | CTD_vs_c (SEQ ID NO: 45) | 0.9 ⁽⁵⁾ |
| 2375 (SEQ ID NO: 139) | CTD_vs_d (SEQ ID NO: 47) | ≤0.8 ⁽⁵⁾ |
| 2376 (SEQ ID NO: 141) | CTD_vs_e (SEQ ID NO: 49) | ≤0.8 ⁽⁵⁾ |
| 2377 (SEQ ID NO: 143) | RBD_a (SEQ ID NO: 51) | ≤0.8 ⁽⁵⁾ |
| 2378 (SEQ ID NO: 145) | RBD_b (SEQ ID NO: 53) | ≤0.8 ⁽⁵⁾ |
| 2380 (SEQ ID NO: 149) | RBD_d (SEQ ID NO: 55) | ≤0.8 ⁽⁵⁾ |
| 2381 (SEQ ID NO: 151) | RBD_e (SEQ ID NO: 57) | 14.5 ⁽⁵⁾ |
| 2393 (SEQ ID NO: 155) | (RBD-tight)₂ (SEQ ID NO: 173) | ≤0.8 ⁽⁷⁾ |
| 2394 (SEQ ID NO: 157) | (RBD-extended)₂ (SEQ ID NO: | 7.4 ⁽⁷⁾ |
| | 177) | |
| 2395 ((SEQ ID NO: 159) | (RBD)₂ (SEQ ID NO: 181) | ≤0.8 ⁽⁷⁾ |
| 2397-2400 (SEQ ID NO: 161) | (CTD_short_d)₂ (SEQ ID NO: 183) | 32.2 ⁽⁴⁾/53.0 (±4) ⁽⁷⁾ |
| 2401-2404 (SEQ ID NO: 163) | (CTD_short_i)₂ (SEQ ID NO: 185) | 88.3 ⁽⁴⁾/144.7 (±5.5) ⁽⁷⁾ |
| 2421 (SEQ ID NO: 165) | [(CTD_short_i)₂-Fc; D2 mutations | 79.5 ⁽⁴⁾ |
| | for 501.V2 variant, K417N, | |
| | E484K, N501Y] (SEQ ID NO: 187) | |
| 2423 (SEQ ID NO: 167) | [(CTD_short_i)₂-Fc; D2 mutations | 55.2 ⁽⁴⁾ |
| | for hybrid P.1 and CAL.20C | |
| | variants; K417T, L452R, E484K, | |
| | N501Y] (SEQ ID NO: 189) | |
| 2435 (SEQ ID NO: 169) | [(CTD_short_i)₂-Fc; D1 and D2 | 61.2 ⁽⁴⁾ |
| | mutations for hybrid P.1 and | |
| | CAL.20C variants; K417T, L452R, | |
| | E484K, N501Y] (SEQ ID NO: 191) | |

⁽⁴⁾ 4-day expression,
⁽⁵⁾ 5-day expression,
⁽⁷⁾ 7-day expression

Based on the desire to develop methods to increase the relative antigen content of a protein molecule, in-series concatemers of RBD-containing protein fragments translationally fused to an Fc. CTD_short_d and CTD_short_i (SEQ ID NOs: 29 and 39, respectively) were prepared and selected for in-series expression given their high-level, protease resistant expression as monomers. When conjoined with a serine-glycine linker and expressed in CHO cells, the resulting double-domain proteins expressed at similar levels as the monomer constructs. In addition, the in-series design retained the molecular resistance to proteolysis during expression and purification. Therefore, by combining intramolecular dimerization driven by Fc interactions with in-series domain expression, it was possible to go from a single SARS-CoV-2 antigen fragment per molecule to having 4 (and potentially more) per molecule. The higher antigen content in conjunction with robust expression levels and protein stability provides a suitable framework for an immunogen to be used for vaccine and boost applications.

As an example of this approach, double-domain Fc-fusion constructs (i.e., recombinant polypeptides containing two immunogenic fragments connected to an antibody Fc region) containing amino acid mutations within one or both of the domains were prepared. The mutations used were either from a single virus variant or a hybrid of two virus variants. Incorporation of the mutations into the double-domain-Fc wild-type molecule (strain 2401) depicted in FIG. 3 followed by expression in CHO cells resulted in intact, soluble protein with similar yields to the wild-type amino acid sequence. These results support the concept that this multi-domain Fc-fusion platform provides a robust scaffold for the incorporation and expression molecules that reflect new variant mutations. This provides a robust, timely and cost-effective system to adapt vaccine composition to meet the needs to mitigate evolving variants. A vaccine containing strain 2401 was subsequently tested in Rhesus macque monkeys.

Vaccination: Vaccination and boosters to nCOV-2 double dimer (Lytic Solutions Strain #2401, SEQ ID NO: 163, administered to all animals except for those indicated to be administered the nCOV-2 quadruple mutant vaccine) or nCOV-2 quadruple mutant vaccine (Lytic Solutions Strain #2435, SEQ ID NO: 169, referred to in Table 2C as “Variant COVID Vaccine” in Adjuvant & Dose column) were performed on Rhesus macaque monkeys by the following methodology. 50, 25, or 12.5 micrograms (these numbers are used to define dosage) of nCOV-2 protein was mixed with either AS03 (Invivogen catalog vac-as03-10) or alum (Invivogen catalog vac-alu-250) as a 1:1 volume mixture protein: adjuvant. For 50 ug doses 500 ul of each protein and adjuvant were used, for 25 ug and 12.5 ug 250 ul of each protein and adjuvant were used. Dosages were split and injected intramuscularly into left and right thighs at the time of initial vaccination and at booster vaccination. Initial vaccination day was designated day 0. Boosters were given 28 day post day 0 unless otherwise noted. Serum samples were taken prior to initial vaccination on day 0, day 14, day 28 prior to booster vaccination, and day 42 and day 56 were 2/4 weeks post booster injection. Therefore data from 14 and 28 (immunization plus 2 weeks and immunization plus 4 weeks, respectively) days are specific to a single dose of vaccine whereas samples from day 42 and day 56 are 2 doses of vaccine (booster plus 2 weeks, and booster plus 4 weeks respectively). Each animal received the same dosage of protein and adjuvant for the booster dose that they received in the primary dose.

Analysis of antibody titers were performed at Intuitive Biosciences, Madison WI on their proprietary ELISA system (918 Deming Way, Suite 100, Madison WI 53719 USA). Serum was serially diluted in CSA buffer (Intuitive product no. 7-1037) to dilutions of 1:100, 1:1000, 1:10,000, 1:100,000, and 1:1,000,000. Analysis was performed at and by Intuitive Biosciences. ELISA units are measured as density on their platform with a maximum signal of approximately 45,000-50,000 counts. Titer signals were determined from dilutions that yielded signals less than ⅓rd maximal signal. Fifteen animals were used to determine vaccine/booster efficacy. Animal names are codes generated for each animal at the primate facility at UW-Madison. All animals were assayed through day 56 post initial vaccination. 50, 25, and 12.5 ug doses gave similar titers for day 28. Some animals were followed past the study design point of 56 days to various time points up to 23 weeks post initial vaccine (due to continued potency of vaccine response). Data is summarized in Tables 2A, 2B, and 2C. It is noted that, although some of the Plate Sample ID numbers are overlapping, e.g., Tables 2A and 2B both have a row with a plate sample ID of 59, this is merely an artifact of the data collection process, such that rows with the same Plate Sample ID represent independently collected data points.

50 ug, 25 ug, 12.5 ug: These data show that relatively low dosages of the tested constructs, including 12.5 ug, still elicited sufficient immune stimulation and/or boosting. Such low amounts of protein per vaccine dose allow for an increased number of active doses to be produced per liter of cell culture. As production levels increase, not only do cost of goods go down, but the timeframe to produce large numbers of doses can be decreased in comparison to vaccine compositions requiring higher amounts of protein per dose to provide sufficient immune protection. This is particularly relevant to providing immune protection against any newly-arising SARS-COV-2 strains.

The assembly of four point-mutations into a single RBD polypeptide was undertaken prior to public disclosure of the Delta mutant isolated first in India. Vaccine LS2435 (SEQ ID NO: 169) contains mutations in four sites that reflect mutation that evolved from new variants identified from the UK, South Africa, Brazil and southern California. Convergent evolution of mutations in new variants lead this to be an attractive approach of stacking multiple mutations in one construct to represent multiple variants, such as seen in the Delta variant. Each RBD point mutation was determined to add virulency through either immune system avoidance or enhanced viral entry or production of higher viral loads (or a combination thereof). The identified mutations were combined to develop a vaccine that could address each mutation and immune epitope singly or in combination. When evolutionary boundaries are considered, it became clear that mutational stacking was likely to take place through either recombination of previous viral strains, or additional mutations stacked onto previous viral strains that enhanced virulence/transmission. Considering the mutation-stacked SARS COV-2 strains can evade the immune response as well as generate a more potent viral titer, having a vaccine that displays high levels of antibody and T-cell potency is a major advantage over previous COVID-19 vaccines which can provide less potent immune responses to these mutants. The SARS COV-2 mutant-containing vaccine LS2435 demonstrates highly potent immune stimulation and antibody production in Rhesus macaques. In fact, the levels of antibodies achieved with the mutant vaccine are similar to responses seen from the tested wild-type vaccine, i.e., LS2401.

TABLE 2A

| | | | | | Animal - | |
|---|---|---|---|---|---|---|
| Plate | | | | | Adjuvant & | Days post |
| Sample ID | Spike S1 | Spike S2 | Nucleocapsid | Dilution | Dosage | injection |
| 59 | 14 | 217 | 296 | 1:100 | BH56 - | 0 |
| | | | | | AS03 50 ug | |
| 60 | 19 | 21 | 418 | 1:1,000 | BH56 - | 0 |
| | | | | | AS03 50 ug | |
| 61 | 0 | 30 | 100 | 1:10,000 | BH56 - | 0 |
| | | | | | AS03 50 ug | |
| 62 | 44982 | 343 | 489 | 1:100 | BH56 - | 14 |
| | | | | | AS03 50 ug | |
| 63 | 31862 | 49 | 445 | 1:1,000 | BH56 - | 14 |
| | | | | | AS03 50 ug | |
| 64 | 3227 | 28 | 276 | 1:10,000 | BH56 - | 14 |
| | | | | | AS03 50 ug | |
| 65 | 47053 | 507 | 642 | 1:100 | BH56 - | 28 |
| | | | | | AS03 50 ug | |
| 66 | 35647 | 42 | 46 | 1:1,000 | BH56 - | 28 |
| | | | | | AS03 50 ug | |
| 67 | 9818 | 57 | 290 | 1:10,000 | BH56 - | 28 |
| | | | | | AS03 50 ug | |
| 68 | 34 | 116 | 508 | 1:100 | BC43 - | 0 |
| | | | | | Alum 50 ug | |
| 69 | 0 | 30 | 291 | 1:1,000 | BC43 - | 0 |
| | | | | | Alum 50 ug | |
| 70 | 22 | 52 | 1 | 1:10,000 | BC43 - | 0 |
| | | | | | Alum 50 ug | |
| 71 | 36334 | 67 | 208 | 1:100 | BC43 - | 14 |
| | | | | | Alum 50 ug | |
| 72 | 14375 | 720 | 553 | 1:1,000 | BC43 - | 14 |
| | | | | | Alum 50 ug | |
| 73 | 234 | 0 | 0 | 1:10,000 | BC43 - | 14 |
| | | | | | Alum 50 ug | |
| 74 | 46731 | 20 | 36 | 1:100 | BC43 - | 28 |
| | | | | | Alum 50 ug | |
| 75 | 39535 | 56 | 258 | 1:1,000 | BC43 - | 28 |
| | | | | | Alum 50 ug | |
| 76 | 5458 | 17 | 1 | 1:10,000 | BC43 - | 28 |
| | | | | | Alum 50 ug | |
| 77 | 0 | 97 | 318 | 1:100 | BH95 - | 0 |
| | | | | | Alum 50 ug | |
| 78 | 0 | 66 | 146 | 1:1,000 | BH95 - | 0 |
| | | | | | Alum 50 ug | |
| 79 | 1 | 12 | 23 | 1:10,000 | BH95 - | 0 |
| | | | | | Alum 50 ug | |
| 80 | 42119 | 63 | 259 | 1:100 | BH95 - | 14 |
| | | | | | Alum 50 ug | |
| 81 | 30356 | 59 | 644 | 1:1,000 | BH95 - | 14 |
| | | | | | Alum 50 ug | |
| 82 | 451 | 0 | 0 | 1:10,000 | BH95 - | 14 |
| | | | | | Alum 50 ug | |
| 83 | 49046 | 156 | 63 | 1:100 | BH95 - | 28 |
| | | | | | Alum 50 ug | |
| 84 | 40830 | 0 | 187 | 1:1,000 | BH95 - | 28 |
| | | | | | Alum 50 ug | |
| 85 | 7282 | 28 | 78 | 1:10,000 | BH95 - | 28 |
| | | | | | Alum 50 ug | |
| 86 | 40 | 55 | 557 | 1:100 | BG86 - | 0 |
| | | | | | AS03 50 ug | |
| 87 | 40 | 93 | 766 | 1:1,000 | BG86 - | 0 |
| | | | | | AS03 50 ug | |
| 88 | 0 | 27 | 229 | 1:10,000 | BG86 - | 0 |
| | | | | | AS03 50 ug | |
| 89 | 27694 | 144 | 618 | 1:100 | BG86 - | 14 |
| | | | | | AS03 50 ug | |
| 90 | 436 | 114 | 275 | 1:1,000 | BG86 - | 14 |
| | | | | | AS03 50 ug | |
| 91 | 243 | 23 | 91 | 1:10,000 | BG86 - | 14 |
| | | | | | AS03 50 ug | |
| 92 | 46656 | 85 | 618 | 1:100 | BG86 - | 28 |
| | | | | | AS03 50 ug | |
| 93 | 38813 | 84 | 527 | 1:1,000 | BG86 - | 28 |
| | | | | | AS03 50 ug | |
| 94 | 13019 | 61 | 65 | 1:10,000 | BG86 - | 28 |
| | | | | | AS03 50 ug | |
| Neg Cntl | 46 | 475 | 986 | | | |
| Pos Cntl | 44473 | 39802 | 31871 | | | |

TABLE 2B

| | | | | | Animal - | Time after |
|---|---|---|---|---|---|---|
| Plate | | | | | Adjuvant & | initial |
| Sample ID | Spike S1 | Spike S2 | Nucleocapsid | Dilution | Dosage | vaccination |
| 10 | 47883 | 0 | 96 | 1:10,000 | BC43 - | Day 42 |
| | | | | | Alum 50 ug | |
| 11 | 25917 | 0 | 0 | 1:100,000 | BC43 - | Day 42 |
| | | | | | Alum 50 ug | |
| 12 | 3597 | 14 | 0 | 1:1,000,000 | BC43 - | Day 42 |
| | | | | | Alum 50 ug | |
| 13 | 45232 | 0 | 60 | 1:10,000 | BC43 - | Day 56 |
| | | | | | Alum 50 ug | |
| 14 | 16743 | 0 | 0 | 1:100,000 | BC43 - | Day 56 |
| | | | | | Alum 50 ug | |
| 15 | 2099 | 0 | 0 | 1:1,000,000 | BC43 - | Day 56 |
| | | | | | Alum 50 ug | |
| 16 | 47513 | 156 | 1 | 1:10,000 | BH56 - | Day 42 |
| | | | | | AS03 50 ug | |
| 17 | 24243 | 31 | 21 | 1:100,000 | BH56 - | Day 42 |
| | | | | | AS03 50 ug | |
| 18 | 3976 | 11 | 0 | 1:1,000,000 | BH56 - | Day 42 |
| | | | | | AS03 50 ug | |
| 19 | 45909 | 0 | 31 | 1:10,000 | BH56 - | Day 56 |
| | | | | | AS03 50 ug | |
| 20 | 18140 | 26 | 5 | 1:100,000 | BH56 - | Day 56 |
| | | | | | AS03 50 ug | |
| 21 | 1184 | 2 | 0 | 1:1,000,000 | BH56 - | Day 56 |
| | | | | | AS03 50 ug | |
| 22 | 44095 | 53 | 0 | 1:10,000 | BH95 - | Day 42 |
| | | | | | Alum 50 ug | |
| 23 | 20940 | 26 | 0 | 1:100,000 | BH95 - | Day 42 |
| | | | | | Alum 50 ug | |
| 24 | 1845 | 2 | 28 | 1:1,000,000 | BH95 - | Day 42 |
| | | | | | Alum 50 ug | |
| 25 | 45464 | 67 | 177 | 1:10,000 | BH95 - | Day 56 |
| | | | | | Alum 50 ug | |
| 26 | 17409 | 21 | 5 | 1:100,000 | BH95 - | Day 56 |
| | | | | | Alum 50 ug | |
| 27 | 1281 | 22 | 0 | 1:1,000,000 | BH95 - | Day 56 |
| | | | | | Alum 50 ug | |
| 28 | 48319 | 71 | 0 | 1:10,000 | BC11- | Day 42 |
| | | | | | AS03 50 ug | |
| 29 | 30010 | 10 | 0 | 1:100,000 | BC11- | Day 42 |
| | | | | | AS03 50 ug | |
| 30 | 671 | 14 | 0 | 1:1,000,000 | BC11- | Day 42 |
| | | | | | AS03 50 ug | |
| 31 | 16796 | 4 | 0 | 1:10,000 | BG86 - | Single dose 8 |
| | | | | | AS03 50 ug | weeks |
| 32 | 1550 | 11 | 56 | 1:100,000 | BG86 - | Single dose 8 |
| | | | | | AS03 50 ug | weeks |
| 33 | 164 | 17 | 6 | 1:1,000,000 | BG86 - | Single dose 8 |
| | | | | | AS03 50 ug | weeks |
| 34 | 0 | 64 | 15 | 1:1,000 | BI37 - | Day 0 |
| | | | | | Alum 25 ug | |
| 35 | 2 | 5 | 0 | 1:10,000 | BI37 - | Day 0 |
| | | | | | Alum 25 ug | |
| 36 | 8 | 6 | 25 | 1:100,000 | BI37 - | Day 0 |
| | | | | | Alum 25 ug | |
| 37 | 40318 | 112 | 0 | 1:1,000 | BI37 - | Day 14 |
| | | | | | Alum 25 ug | |
| 38 | 4456 | 35 | 28 | 1:10,000 | BI37 - | Day 14 |
| | | | | | Alum 25 ug | |
| 39 | 655 | 35 | 17 | 1:100,000 | BI37 - | Day 14 |
| | | | | | Alum 25 ug | |
| 40 | 49137 | 127 | 31 | 1:1,000 | BI37 - | Day 28 |
| | | | | | Alum 25 ug | |
| 41 | 32611 | 29 | 2 | 1:10,000 | BI37 - | Day 28 |
| | | | | | Alum 25 ug | |
| 42 | 5526 | 34 | 0 | 1:100,000 | BI37 - | Day 28 |
| | | | | | Alum 25 ug | |
| 43 | 0 | 56 | 15 | 1:1,000 | BK78 - | Day 0 |
| | | | | | AS03 25 ug | |
| 44 | 36 | 46 | 27 | 1:10,000 | BK78 - | Day 0 |
| | | | | | AS03 25 ug | |
| 45 | 45 | 34 | 0 | 1:100,000 | BK78 - | Day 0 |
| | | | | | AS03 25 ug | |
| 46 | 26462 | 49 | 87 | 1:1,000 | BK78 - | Day 14 |
| | | | | | AS03 25 ug | |
| 47 | 965 | 122 | 29 | 1:10,000 | BK78 - | Day 14 |
| | | | | | AS03 25 ug | |
| 48 | 92 | 28 | 37 | 1:100,000 | BK78 - | Day 14 |
| | | | | | AS03 25 ug | |
| 49 | 36563 | 67 | 31 | 1:1,000 | BK78 - | Day 28 |
| | | | | | AS03 25 ug | |
| 50 | 6352 | 34 | 17 | 1:10,000 | BK78 - | Day 28 |
| | | | | | AS03 25 ug | |
| 51 | 917 | 34 | 60 | 1:100,000 | BK78 - | Day 28 |
| | | | | | AS03 25 ug | |
| 52 | 1 | 0 | 160 | 1:1,000 | BM52 - | Day 0 |
| | | | | | AS03 12.5 ug | |
| 53 | 15 | 64 | 60 | 1:10,000 | BM52 - | Day 0 |
| | | | | | AS03 12.5 ug | |
| 54 | 12 | 79 | 49 | 1:100,000 | BM52 - | Day 0 |
| | | | | | AS03 12.5 ug | |
| 55 | 17806 | 684 | 315 | 1:1,000 | BM52 - | Day 14 |
| | | | | | AS03 12.5 ug | |
| 56 | 380 | 147 | 35 | 1:10,000 | BM52 - | Day 14 |
| | | | | | AS03 12.5 ug | |
| 57 | 96 | 32 | 18 | 1:100,000 | BM52 - | Day 14 |
| | | | | | AS03 12.5 ug | |
| 58 | 39459 | 440 | 29 | 1:1,000 | BM52 - | Day 28 |
| | | | | | AS03 12.5 ug | |
| 59 | 4104 | 56 | 12 | 1:10,000 | BM52 - | Day 28 |
| | | | | | AS03 12.5 ug | |
| 60 | 467 | 13 | 26 | 1:100,000 | BM52 - | Day 28 |
| | | | | | AS03 12.5 ug | |
| 61 | 19 | 51 | 0 | 1:1,000 | BC31 - | Day 0 |
| | | | | | Alum 12.5 ug | |
| 62 | 0 | 0 | 2 | 1:10,000 | BC31 - | Day 0 |
| | | | | | Alum 12.5 ug | |
| 63 | 0 | 74 | 30 | 1:100,000 | BC31 - | Day 0 |
| | | | | | Alum 12.5 ug | |
| 64 | 9436 | 123 | 40 | 1:1,000 | BC31 - | Day 14 |
| | | | | | Alum 12.5 ug | |
| 65 | 426 | 27 | 0 | 1:10,000 | BC31 - | Day 14 |
| | | | | | Alum 12.5 ug | |
| 66 | 8 | 31 | 0 | 1:100,000 | BC31 - | Day 14 |
| | | | | | Alum 12.5 ug | |
| 67 | 36234 | 0 | 36 | 1:1,000 | BC31 - | Day 28 |
| | | | | | Alum 12.5 ug | |
| 68 | 7220 | 24 | 14 | 1:10,000 | BC31 - | Day 28 |
| | | | | | Alum 12.5 ug | |
| 69 | 160 | 9 | 8 | 1:100,000 | BC31 - | Day 28 |
| | | | | | Alum 12.5 ug | |
| Neg Cntl | 221 | 1837 | 90 | | | |
| Neg Cntl | 12 | 46 | 10 | | | |
| Pos Cntl | 49197 | 44161 | 45329 | | | |
| Pos Cntl | 47017 | 41224 | 46778 | | | |

TABLE 2C

| | | | | | | Days after | |
|---|---|---|---|---|---|---|---|
| Plate | | | | | | initial | Adjuvant |
| Sample ID | Spike S1 | Spike S2 | Nucleocapsid | Dilution | Animal | injection | & Dose |
| 1A | 15544 | 333 | 104 | 1:10,000 | BG86 | 84 | AS03 50 ug - |
| | | | | | | | single dose |
| 1B | 1751 | 277 | 255 | 1:100,000 | BG86 | 84 | AS03 50 ug - |
| | | | | | | | single dose |
| 1C | 481 | 276 | 123 | 1:1,000,000 | BG86 | 84 | AS03 50 ug - |
| | | | | | | | single dose |
| 2A | 35682 | 375 | 0 | 1:10,000 | BH56 | 84 | AS03 50 ug |
| 2B | 9450 | 313 | 259 | 1:100,000 | BH56 | 84 | AS03 50 ug |
| 2C | 1697 | 329 | 253 | 1:1,000,000 | BH56 | 84 | AS03 50 ug |
| 3A | 39567 | 327 | 368 | 1:10,000 | BC43 | 84 | Alum 50 ug |
| 3B | 9891 | 95 | 124 | 1:100,000 | BC43 | 84 | Alum 50 ug |
| 3C | 1824 | 272 | 0 | 1:1,000,000 | BC43 | 84 | Alum 50 ug |
| 4A | 37479 | 135 | 718 | 1:10,000 | BH95 | 84 | Alum 50 ug |
| 4B | 8790 | 161 | 143 | 1:100,000 | BH95 | 84 | Alum 50 ug |
| 4C | 1619 | 201 | 132 | 1:1,000,000 | BH95 | 84 | Alum 50 ug |
| 6A | 8417 | 127 | 454 | 1:10,000 | BC11 | 14 | AS03 50 ug |
| 6B | 849 | 179 | 71 | 1:100,000 | BC11 | 14 | AS03 50 ug |
| 6C | 265 | 381 | 273 | 1:1,000,000 | BC11 | 14 | AS03 50 ug |
| 7A | 13381 | 607 | 664 | 1:10,000 | BC11 | 28 | AS03 50 ug |
| 7B | 803 | 176 | 81 | 1:100,000 | BC11 | 28 | AS03 50 ug |
| 7C | 341 | 251 | 275 | 1:1,000,000 | BC11 | 28 | AS03 50 ug |
| 8A | 45422 | 341 | 285 | 1:10,000 | BC11 | 56 | AS03 50 ug |
| 8B | 18249 | 173 | 165 | 1:100,000 | BC11 | 56 | AS03 50 ug |
| 8C | 3752 | 213 | 357 | 1:1,000,000 | BC11 | 56 | AS03 50 ug |
| 9A | 42056 | 341 | 292 | 1:10,000 | BC11 | 84 | AS03 50 ug |
| 9B | 14271 | 316 | 163 | 1:100,000 | BC11 | 84 | AS03 50 ug |
| 9C | 2393 | 256 | 129 | 1:1,000,000 | BC11 | 84 | AS03 50 ug |
| 10A | 44288 | 237 | 150 | 1:10,000 | BI37 | 42 | Alum 25 ug |
| 10B | 19432 | 189 | 119 | 1:100,000 | BI37 | 42 | Alum 25 ug |
| 10C | 4354 | 378 | 231 | 1:1,000,000 | BI37 | 42 | Alum 25 ug |
| 11A | 43800 | 239 | 35 | 1:10,000 | BI37 | 56 | Alum 25 ug |
| 11B | 15355 | 138 | 75 | 1:100,000 | BI37 | 56 | Alum 25 ug |
| 11C | 2865 | 337 | 247 | 1:1,000,000 | BI37 | 56 | Alum 25 ug |
| 12A | 38022 | 305 | 18 | 1:10,000 | BI37 | 84 | Alum 25 ug |
| 12B | 11812 | 294 | 86 | 1:100,000 | BI37 | 84 | Alum 25 ug |
| 12C | 1948 | 213 | 243 | 1:1,000,000 | BI37 | 84 | Alum 25 ug |
| 13A | 46998 | 398 | 167 | 1:10,000 | BK78 | 42 | AS03 25 ug |
| 13B | 24787 | 267 | 116 | 1:100,000 | BK78 | 42 | AS03 25 ug |
| 13C | 6808 | 442 | 0 | 1:1,000,000 | BK78 | 42 | AS03 25 ug |
| 14A | 45244 | 441 | 0 | 1:10,000 | BK78 | 56 | AS03 25 ug |
| 14B | 18294 | 209 | 84 | 1:100,000 | BK78 | 56 | AS03 25 ug |
| 14C | 3830 | 484 | 256 | 1:1,000,000 | BK78 | 56 | AS03 25 ug |
| 15A | 41106 | 478 | 0 | 1:10,000 | BK78 | 84 | AS03 25 ug |
| 15B | 14414 | 275 | 83 | 1:100,000 | BK78 | 84 | AS03 25 ug |
| 15C | 3328 | 435 | 269 | 1:1,000,000 | BK78 | 84 | AS03 25 ug |
| 16A | 46111 | 524 | 779 | 1:10,000 | BM52 | 42 | AS03 12.5 ug |
| 16B | 24586 | 113 | 300 | 1:100,000 | BM52 | 42 | AS03 12.5 ug |
| 16C | 7160 | 223 | 156 | 1:1,000,000 | BM52 | 42 | AS03 12.5 ug |
| 17A | 41733 | 328 | 527 | 1:10,000 | BM52 | 56 | AS03 12.5 ug |
| 17B | 18140 | 284 | 157 | 1:100,000 | BM52 | 56 | AS03 12.5 ug |
| 17C | 3499 | 222 | 380 | 1:1,000,000 | BM52 | 56 | AS03 12.5 ug |
| 18A | 36263 | 995 | 242 | 1:10,000 | BM52 | 84 | AS03 12.5 ug |
| 18B | 11912 | 525 | 277 | 1:100,000 | BM52 | 84 | AS03 12.5 ug |
| 18C | 1679 | 190 | 301 | 1:1,000,000 | BM52 | 84 | AS03 12.5 ug |
| 19A | 43970 | 345 | 37 | 1:10,000 | BC31 | 42 | Alum 12.5 ug |
| 19B | 20155 | 255 | 25 | 1:100,000 | BC31 | 42 | Alum 12.5 ug |
| 19C | 3849 | 321 | 268 | 1:1,000,000 | BC31 | 42 | Alum 12.5 ug |
| 20A | 40779 | 264 | 0 | 1:10,000 | BC31 | 56 | Alum 12.5 ug |
| 20B | 18417 | 298 | 196 | 1:100,000 | BC31 | 56 | Alum 12.5 ug |
| 20C | 2169 | 261 | 24 | 1:1,000,000 | BC31 | 56 | Alum 12.5 ug |
| 21A | 32251 | 89 | 0 | 1:10,000 | BC31 | 84 | Alum 12.5 ug |
| 21B | 9977 | 314 | 197 | 1:100,000 | BC31 | 84 | Alum 12.5 ug |
| 21C | 1521 | 384 | 213 | 1:1,000,000 | BC31 | 84 | Alum 12.5 ug |
| 22A | 43 | 249 | 129 | 1:10,000 | BM21 | 0 | AS03 12.5 ug |
| 22B | 267 | 215 | 186 | 1:100,000 | BM21 | 0 | AS03 12.5 ug |
| 22C | 225 | 270 | 141 | 1:1,000,000 | BM21 | 0 | AS03 12.5 ug |
| 23A | 2694 | 331 | 96 | 1:10,000 | BM21 | 14 | AS03 12.5 ug |
| 23B | 621 | 202 | 190 | 1:100,000 | BM21 | 14 | AS03 12.5 ug |
| 23C | 297 | 339 | 225 | 1:1,000,000 | BM21 | 14 | AS03 12.5 ug |
| 24A | 5672 | 361 | 75 | 1:10,000 | BM21 | 28 | AS03 12.5 ug |
| 24B | 905 | 39 | 168 | 1:100,000 | BM21 | 28 | AS03 12.5 ug |
| 24C | 91 | 129 | 139 | 1:1,000,000 | BM21 | 28 | AS03 12.5 ug |
| 25A | 45613 | 555 | 118 | 1:10,000 | BM21 | 49 | AS03 12.5 ug |
| 25B | 23568 | 513 | 244 | 1:100,000 | BM21 | 49 | AS03 12.5 ug |
| 25C | 4134 | 293 | 248 | 1:1,000,000 | BM21 | 49 | AS03 12.5 ug |
| 26A | 39959 | 429 | 19 | 1:10,000 | BM21 | 63 | AS03 12.5 ug |
| 26B | 15663 | 309 | 161 | 1:100,000 | BM21 | 63 | AS03 12.5 ug |
| 26C | 2263 | 240 | 273 | 1:1,000,000 | BM21 | 63 | AS03 12.5 ug |
| 27A | 34745 | 371 | 47 | 1:10,000 | BM21 | 91 | AS03 12.5 ug |
| 27B | 9926 | 402 | 121 | 1:100,000 | BM21 | 91 | AS03 12.5 ug |
| 27C | 1197 | 299 | 380 | 1:1,000,000 | BM21 | 91 | AS03 12.5 ug |
| 28A | 879 | 302 | 80 | 1:10,000 | BD82 | 0 | Alum 12.5 ug |
| 28B | 538 | 470 | 120 | 1:100,000 | BD82 | 0 | Alum 12.5 ug |
| 28C | 591 | 135 | 129 | 1:1,000,000 | BD82 | 0 | Alum 12.5 ug |
| 29A | 3764 | 90 | 0 | 1:10,000 | BD82 | 14 | Alum 12.5 ug |
| 29B | 532 | 397 | 227 | 1:100,000 | BD82 | 14 | Alum 12.5 ug |
| 29C | 307 | 583 | 267 | 1:1,000,000 | BD82 | 14 | Alum 12.5 ug |
| 30A | 9816 | 337 | 84 | 1:10,000 | BD82 | 21 | Alum 12.5 ug |
| 30B | 2407 | 371 | 169 | 1:100,000 | BD82 | 21 | Alum 12.5 ug |
| 30C | 285 | 462 | 245 | 1:1,000,000 | BD82 | 21 | Alum 12.5 ug |
| 31A | 41086 | 344 | 128 | 1:10,000 | BD82 | 35 | Alum 12.5 ug |
| 31B | 19882 | 159 | 219 | 1:100,000 | BD82 | 35 | Alum 12.5 ug |
| 31C | 2912 | 333 | 73 | 1:1,000,000 | BD82 | 35 | Alum 12.5 ug |
| 32A | 36345 | 245 | 57 | 1:10,000 | BD82 | 49 | Alum 12.5 ug |
| 32B | 13460 | 469 | 264 | 1:100,000 | BD82 | 49 | Alum 12.5 ug |
| 32C | 2585 | 149 | 9 | 1:1,000,000 | BD82 | 49 | Alum 12.5 ug |
| 33A | 27977 | 400 | 185 | 1:10,000 | BD82 | 77 | Alum 12.5 ug |
| 33B | 7948 | 288 | 80 | 1:100,000 | BD82 | 77 | Alum 12.5 ug |
| 33C | 904 | 262 | 28 | 1:1,000,000 | BD82 | 77 | Alum 12.5 ug |
| 34A | 62 | 274 | 150 | 1:10,000 | BE83 | 0 | Alum 25 ug |
| 34B | 385 | 232 | 217 | 1:100,000 | BE83 | 0 | Alum 25 ug |
| 34C | 142 | 306 | 292 | 1:1,000,000 | BE83 | 0 | Alum 25 ug |
| 35A | 3232 | 69 | 94 | 1:10,000 | BE83 | 14 | Alum 25 ug |
| 35B | 804 | 343 | 34 | 1:100,000 | BE83 | 14 | Alum 25 ug |
| 35C | 129 | 222 | 161 | 1:1,000,000 | BE83 | 14 | Alum 25 ug |
| 36A | 8300 | 150 | 58 | 1:10,000 | BE83 | 21 | Alum 25 ug |
| 36B | 1700 | 295 | 153 | 1:100,000 | BE83 | 21 | Alum 25 ug |
| 36C | 266 | 285 | 111 | 1:1,000,000 | BE83 | 21 | Alum 25 ug |
| 37A | 38645 | 368 | 0 | 1:10,000 | BE83 | 35 | Alum 25 ug |
| 37B | 13433 | 320 | 151 | 1:100,000 | BE83 | 35 | Alum 25 ug |
| 37C | 3155 | 306 | 112 | 1:1,000,000 | BE83 | 35 | Alum 25 ug |
| 38A | 35954 | 375 | 67 | 1:10,000 | BE83 | 49 | Alum 25 ug |
| 38B | 14846 | 190 | 89 | 1:100,000 | BE83 | 49 | Alum 25 ug |
| 38C | 3553 | 339 | 246 | 1:1,000,000 | BE83 | 49 | Alum 25 ug |
| 39A | 23360 | 373 | 0 | 1:10,000 | BE83 | 77 | Alum 25 ug |
| 39B | 7836 | 72 | 31 | 1:100,000 | BE83 | 77 | Alum 25 ug |
| 40A | 12128 | 194 | 190 | 1:10,000 | BG86 | 105 | AS03 50 ug - |
| | | | | | | | single dose |
| 40B | 2292 | 161 | 159 | 1:100,000 | BG86 | 105 | AS03 50 ug - |
| | | | | | | | single dose |
| 40C | 261 | 187 | 191 | 1:1,000,000 | BG86 | 105 | AS03 50 ug - |
| | | | | | | | single dose |
| 41A | 10969 | 161 | 0 | 1:10,000 | BG86 | 139 | AS03 50 ug - |
| | | | | | | | single dose |
| 41B | 1875 | 219 | 181 | 1:100,000 | BG86 | 139 | AS03 50 ug - |
| | | | | | | | single dose |
| 41C | 992 | 837 | 705 | 1:1,000,000 | BG86 | 139 | AS03 50 ug- |
| | | | | | | | single dose |
| 42A | 176 | 57 | 120 | 1:10,000 | BG86 | MILK 1 | AS03 50 ug - |
| | | | | | | month post | single dose |
| | | | | | | birth | |
| 42B | 567 | 216 | 123 | 1:100,000 | BG86 | MILK 1 | AS03 50 ug - |
| | | | | | | month post | single dose |
| | | | | | | birth | |
| 42C | 217 | 283 | 153 | 1:1,000,000 | BG86 | MILK 1 | AS03 50 ug - |
| | | | | | | month post | single dose |
| | | | | | | birth | |
| 43A | 9055 | 140 | 149 | 1:10,000 | BP49 | Birth Serum | no vaccine |
| | | | | | Mother is | | |
| | | | | | BG86 | | |
| 43B | 1410 | 121 | 235 | 1:100,000 | BP49 | Birth Serum | no vaccine |
| | | | | | Mother is | | |
| | | | | | BG86 | | |
| 43C | 388 | 311 | 152 | 1:1,000,000 | BP49 | Birth Serum | no vaccine |
| | | | | | Mother is | | |
| | | | | | BG86 | | |
| 44A | 4525 | 35 | 0 | 1:10,000 | BP49 | 4 weeks post | no vaccine |
| | | | | | Mother is | birth serum | |
| | | | | | BG86 | | |
| 44B | 1259 | 269 | 40 | 1:100,000 | BP49 | 4 weeks post | no vaccine |
| | | | | | Mother is | birth serum | |
| | | | | | BG86 | | |
| 44C | 286 | 261 | 223 | 1:1,000,000 | BP49 | 4 weeks post | no vaccine |
| | | | | | Mother is | birth serum | |
| | | | | | BG86 | | |
| 45A | 23973 | 304 | 97 | 1:10,000 | BC43 | 162 | Alum 50 ug |
| 45B | 4896 | 272 | 161 | 1:100,000 | BC43 | 162 | Alum 50 ug |
| 45C | 649 | 212 | 208 | 1:1,000,000 | BC43 | 162 | Alum 50 ug |
| 46A | 32326 | 192 | 36 | 1:10,000 | BK78 | 142 | AS03 25 ug |
| 46B | 11039 | 336 | 26 | 1:100,000 | BK78 | 142 | AS03 25 ug |
| 46C | 1694 | 418 | 226 | 1:1,000,000 | BK78 | 142 | AS03 25 ug |
| 47A | 21598 | 206 | 0 | 1:10,000 | BM72 | 120 | AS03 25 ug |
| 47B | 5364 | 274 | 237 | 1:100,000 | BM72 | 120 | AS03 25 ug |
| 47C | 496 | 305 | 222 | 1:1,000,000 | BM72 | 120 | AS03 25 ug |
| 48A | 18031 | 166 | 168 | 1:10,000 | BH95 | 162 | Alum 50 ug |
| 48B | 3372 | 304 | 173 | 1:100,000 | BH95 | 162 | Alum 50 ug |
| 48C | 562 | 350 | 191 | 1:1,000,000 | BH95 | 162 | Alum 50 ug |
| 49A | 31501 | 102 | 147 | 1:10,000 | BC11 | 150 | AS03 50 ug |
| 49B | 8850 | 327 | 191 | 1:100,000 | BC11 | 150 | AS03 50 ug |
| 49C | 1716 | 447 | 257 | 1:1,000,000 | BC11 | 150 | AS03 50 ug |
| 50A | 38099 | 414 | 277 | 1:10,000 | BM52 | 142 | AS03 12.5 ug |
| 50B | 16407 | 288 | 305 | 1:100,000 | BM52 | 142 | AS03 12.5 ug |
| 50C | 3171 | 304 | 136 | 1:1,000,000 | BM52 | 142 | AS03 12.5 ug |
| 51A | 358 | 131 | 191 | 1:10,000 | BJ55 | 0 | None - |
| | | | | | | | Control |
| 51B | 443 | 363 | 357 | 1:100,000 | BJ55 | 0 | None - |
| | | | | | | | Control |
| 51C | 138 | 371 | 117 | 1:1,000,000 | BJ55 | 0 | None - |
| | | | | | | | Control |
| 52A | 19609 | 216 | 139 | 1:10,000 | BI37 | 150 | Alum 25 ug |
| 52B | 4058 | 261 | 214 | 1:100,000 | BI37 | 150 | Alum 25 ug |
| 52C | 465 | 148 | 32 | 1:1,000,000 | BI37 | 150 | Alum 25 ug |
| 53A | 31360 | 392 | 175 | 1:10,000 | BH56 | 169 | AS03 50 ug |
| 53B | 8082 | 202 | 146 | 1:100,000 | BH56 | 169 | AS03 50 ug |
| 53C | 1013 | 342 | 97 | 1:1,000,000 | BH56 | 169 | AS03 50 ug |
| 54A | 20996 | 282 | 110 | 1:10,000 | BE83 | 118 | Alum 25 ug |
| 54B | 4122 | 195 | 75 | 1:100,000 | BE83 | 118 | Alum 25 ug |
| 54C | 534 | 319 | 226 | 1:1,000,000 | BE83 | 118 | Alum 25 ug |
| 55A | 433 | 353 | 151 | 1:10,000 | BI98 | 0 | None - |
| | | | | | | | Control |
| 55B | 290 | 186 | 114 | 1:100,000 | BI98 | 0 | None - |
| | | | | | | | Control |
| 55C | 129 | 297 | 190 | 1:1,000,000 | BI98 | 0 | None - |
| | | | | | | | Control |
| 56A | 21633 | 245 | 129 | 1:10,000 | BI21 | 56 | Variant |
| | | | | | | | COVID |
| | | | | | | | Vaccine |
| | | | | | | | AS03 25 ug |
| 56B | 5705 | 570 | 216 | 1:100,000 | BI21 | 56 | Variant |
| | | | | | | | COVID |
| | | | | | | | Vaccine |
| | | | | | | | AS03 25 ug |
| 56C | 840 | 228 | 155 | 1:1,000,000 | BI21 | 56 | Variant |
| | | | | | | | COVID |
| | | | | | | | Vaccine |
| | | | | | | | AS03 25 ug |
| 57A | 10191 | 173 | 239 | 1:10,000 | BC31 | 148 | Alum 12.5 ug |
| 57B | 1927 | 340 | 170 | 1:100,000 | BC31 | 148 | Alum 12.5 ug |
| 57C | 217 | 480 | 353 | 1:1,000,000 | BC31 | 148 | Alum 12.5 ug |
| 58A | 25739 | 262 | 179 | 1:10,000 | BK44 | 56 | Variant |
| | | | | | | | COVID |
| | | | | | | | Vaccine |
| | | | | | | | AS03 25 ug |
| 58B | 4927 | 278 | 91 | 1:100,000 | BK44 | 56 | Variant |
| | | | | | | | COVID |
| | | | | | | | Vaccine |
| | | | | | | | AS03 25 ug |
| 58C | 1183 | 183 | 113 | 1:1,000,000 | BK44 | 56 | Variant |
| | | | | | | | COVID |
| | | | | | | | Vaccine |
| | | | | | | | AS03 25 ug |
| 59A | 20341 | 177 | 4 | 1:10,000 | BD82 | 118 | Alum 12.5 ug |
| 59B | 3719 | 264 | 238 | 1:100,000 | BD82 | 118 | Alum 12.5 ug |
| 59C | 410 | 377 | 318 | 1:1,000,000 | BD82 | 118 | Alum 12.5 ug |
| 60A | 38868 | 621 | 413 | 1:10,000 | BM52 | 148 | AS03 12.5 ug |
| 60B | 14327 | 303 | 250 | 1:100,000 | BM52 | 148 | AS03 12.5 ug |
| 60C | 1649 | 214 | 132 | 1:1,000,000 | BM52 | 148 | AS03 12.5 ug |
| 61A | 26273 | 155 | 0 | 1:10,000 | BM21 | 125 | AS03 12.5 ug |
| 61B | 5330 | 236 | 108 | 1:100,000 | BM21 | 125 | AS03 12.5 ug |
| 61C | 861 | 340 | 169 | 1:1,000,000 | BM21 | 125 | AS03 12.5 ug |
| 62A | 19650 | 161 | 0 | 1:10,000 | BI21 | 84 | Variant |
| | | | | | | | COVID |
| | | | | | | | Vaccine |
| | | | | | | | AS03 25 ug |
| 62B | 5896 | 307 | 69 | 1:100,000 | BI21 | 84 | Variant |
| | | | | | | | COVID |
| | | | | | | | Vaccine |
| | | | | | | | AS03 25 ug |
| 62C | 747 | 244 | 229 | 1:1,000,000 | BI21 | 84 | Variant |
| | | | | | | | COVID |
| | | | | | | | Vaccine |
| | | | | | | | AS03 25 ug |
| 63A | 20018 | 179 | 39 | 1:10,000 | BK44 | 84 | Variant |
| | | | | | | | COVID |
| | | | | | | | Vaccine |
| | | | | | | | AS03 25 ug |
| 63B | 3860 | 184 | 53 | 1:100,000 | BK44 | 84 | A Variant |
| | | | | | | | COVID |
| | | | | | | | Vaccine |
| | | | | | | | S03 25 ug |
| 63C | 614 | 201 | 54 | 1:1,000,000 | BK44 | 84 | Variant |
| | | | | | | | COVID |
| | | | | | | | Vaccine |
| | | | | | | | AS03 25 ug |
| 66A | 0 | 56 | 131 | 1:10,000 | BK44 | 0 | Variant |
| | | | | | | | COVID |
| | | | | | | | Vaccine |
| | | | | | | | AS03 25 ug |
| 66B | 144 | 450 | 345 | 1:100,000 | BK44 | 0 | Variant |
| | | | | | | | COVID |
| | | | | | | | Vaccine |
| | | | | | | | AS03 25 ug |
| 66C | 224 | 411 | 146 | 1:1,000,000 | BK44 | 0 | Variant |
| | | | | | | | COVID |
| | | | | | | | Vaccine |
| | | | | | | | AS03 25 ug |
| 67A | 1739 | 73 | 60 | 1:10,000 | BK44 | 28 | Variant |
| | | | | | | | COVID |
| | | | | | | | Vaccine |
| | | | | | | | AS03 25 ug |
| 67B | 415 | 306 | 202 | 1:100,000 | BK44 | 28 | Variant |
| | | | | | | | COVID |
| | | | | | | | Vaccine |
| | | | | | | | AS03 25 ug |
| 67C | 19 | 229 | 161 | 1:1,000,000 | BK44 | 28 | Variant |
| | | | | | | | COVID |
| | | | | | | | Vaccine |
| | | | | | | | AS03 25 ug |
| 68A | 25898 | 257 | 0 | 1:10,000 | BK44 | 56 | Variant |
| | | | | | | | COVID |
| | | | | | | | Vaccine |
| | | | | | | | AS03 25 ug |
| 68B | 7243 | 332 | 446 | 1:100,000 | BK44 | 56 | Variant |
| | | | | | | | COVID |
| | | | | | | | Vaccine |
| | | | | | | | AS03 25 ug |
| 68C | 1135 | 174 | 92 | 1:1,000,000 | BK44 | 56 | Variant |
| | | | | | | | COVID |
| | | | | | | | Vaccine |
| | | | | | | | AS03 25 ug |
| 69A | 11 | 215 | 103 | 1:10,000 | BI21 | 0 | Variant |
| | | | | | | | COVID |
| | | | | | | | Vaccine |
| | | | | | | | AS03 25 ug |
| 69B | 95 | 0 | 50 | 1:100,000 | BI21 | 0 | Variant |
| | | | | | | | COVID |
| | | | | | | | Vaccine |
| | | | | | | | AS03 25 ug |
| 69C | 152 | 165 | 102 | 1:1,000,000 | BI21 | 0 | Variant |
| | | | | | | | COVID |
| | | | | | | | Vaccine |
| | | | | | | | AS03 25 ug |
| 70A | 2679 | 232 | 120 | 1:10,000 | BI21 | 28 | Variant |
| | | | | | | | COVID |
| | | | | | | | Vaccine |
| | | | | | | | AS03 25 ug |
| 70B | 429 | 335 | 2 | 1:100,000 | BI21 | 28 | Variant |
| | | | | | | | COVID |
| | | | | | | | Vaccine |
| | | | | | | | AS03 25 ug |
| 70C | 327 | 1562 | 839 | 1:1,000,000 | BI21 | 28 | Variant |
| | | | | | | | COVID |
| | | | | | | | Vaccine |
| | | | | | | | AS03 25 ug |
| 71A | 22367 | 177 | 31 | 1:10,000 | BI21 | 56 | Variant |
| | | | | | | | COVID |
| | | | | | | | Vaccine |
| | | | | | | | AS03 25 ug |
| 71B | 5939 | 281 | 116 | 1:100,000 | BI21 | 56 | Variant |
| | | | | | | | COVID |
| | | | | | | | Vaccine |
| | | | | | | | AS03 25 ug |
| 71C | 841 | 299 | 142 | 1:1,000,000 | BI21 | 56 | Variant |
| | | | | | | | COVID |
| | | | | | | | Vaccine |
| | | | | | | | AS03 25 ug |
| 72A | 85 | 292 | 16 | 1:10,000 | BM72 | 0 | AS03 25 ug |
| 72B | 331 | 449 | 281 | 1:100,000 | BM72 | 0 | AS03 25 ug |
| 72C | 294 | 312 | 139 | 1:1,000,000 | BM72 | 0 | AS03 25 ug |
| 73A | 4015 | 229 | 162 | 1:10,000 | BM72 | 28 | AS03 25 ug |
| 73B | 764 | 251 | 262 | 1:100,000 | BM72 | 28 | AS03 25 ug |
| 73C | 476 | 499 | 165 | 1:1,000,000 | BM72 | 28 | AS03 25 ug |
| 74A | 35753 | 239 | 39 | 1:10,000 | BM72 | 56 | AS03 25 ug |
| 74B | 13297 | 277 | 123 | 1:100,000 | BM72 | 56 | AS03 25 ug |
| 74C | 2518 | 396 | 126 | 1:1,000,000 | BM72 | 56 | AS03 25 ug |

Example 3: Expression construct CDS inserts were made by DNA synthesis and cloned into a C-terminal linker-Fc (human IgG1 Fc) expression vector. Transfection grade plasmid was prepared and used for transient transfection of ExpiCHO or CHO-S cells using the Mirus Bio CHOgro medium and transfection system. Transfection cultures were on the 25 mL scale. After 7-days of post-transfection culturing, protein was affinity purified from the cell medium using Protein A chromatography, washed extensively with PBS (pH 7.2), and eluted with low-pH sodium acetate elution followed by rapid neutralization with TRIS/HCl buffer, pH7.2. Eluted protein was analyzed by spectrophotometric absorbance of 280 nm light. Particularly, the constructs analyzed were CTD-short_g (Wuhan) (SEQ ID NO: 35), and CTD-short_i (Wuhan) (SEQ ID NO: 39), both of which were grown in CHO-S cells. CTD-short_i (BA.4/5) (SEQ ID NO: 713); CTD-short_i (BQ.1.1) (SEQ ID NO: 705); CTD-short_i (BA.2.75) (SEQ ID NO: 714); CTD-short_i (BA.2.75.2) (SEQ ID NO: 715); CTD-short_i (BA.4.6) (SEQ ID NO: 716); and CTD-short_i (XBB) (SEQ ID NO: 709), were all grown in ExpiCHO cells.

TABLE 3
Selected Sequences

| SEQ | | |
|---|---|---|
| ID | | |
| NO: | Description | Sequence |
| 705 | BQ.1.1 | NITNLCPFDEVFNATTFASVYAWNRKRISNCVADYSVLYNFAPFFAFKC |
| | fragment 1 | YGVSPTKLNDLCFTNVYADSFVIRGNEVSQIAPGQTGNIADYNYKLPDD |
| | (AA) | FTGCVIAWNSNKLDSTVGGNYNYRYRLFRKSKLKPFERDISTEIYQAGN |
| | | KPCNGVAGVNCYFPLQSYGFRPTYGVGHQPYRVVVLSFELLHAPATVC |
| | | GPKKSTNLVKNK |
| 706 | BQ.1.1 | NITNLCPFDEVFNATTFASVYAWNRKRISNCVADYSVLYNFAPFFAFKC |
| | fragment 2 | YGVSPTKLNDLCFTNVYADSFVIRGNEVSQIAPGQTGNIADYNYKLPDD |
| | (AA) | FTGCVIAWNSNKLDSTVGGNYNYRYRLFRKSKLKPFERDISTEIYQAGN |
| | | KPCNGVAGVNCYFPLQSYGFRPTYGVGHQPYRVVVLSFELLHAPATVC |
| | | GPKKSTNLV |
| 707 | BQ.1.1 | ITNLCPFDEVFNATTFASVYAWNRKRISNCVADYSVLYNFAPFFAFKCY |
| | fragment 3 | GVSPTKLNDLCFTNVYADSFVIRGNEVSQIAPGQTGNIADYNYKLPDDF |
| | (AA) | TGCVIAWNSNKLDSTVGGNYNYRYRLFRKSKLKPFERDISTEIYQAGNK |
| | | PCNGVAGVNCYFPLQSYGFRPTYGVGHQPYRVVVLSFELLHAPATVCG |
| | | PKKSTNLVKNK |
| 708 | BQ.1.1 | NITNLCPFDEVFNATTFASVYAWNRKRISNCVADYSVLYNFAPFFAFKC |
| | fragment 4 | YGVSPTKLNDLCFTNVYADSFVIRGNEVSQIAPGQTGNIADYNYKLPDD |
| | (AA) | FTGCVIAWNSNKLDSTVGGNYNYRYRLFRKSKLKPFERDISTEIYQAGN |
| | | KPCNGVAGVNCYFPLQSYGFRPTYGVGHQPYRVVVLSFELLHAPATVC |
| | | GPKKSTNLV |
| 709 | XBB | NITNLCPFHEVFNATTFASVYAWNRKRISNCVADYSVLYNFAPFFAFKC |
| | fragment 1 | YGVSPTKLNDLCFTNVYADSFVIRGNEVSQIAPGQTGNIADYNYKLPDD |
| | (AA) | FTGCVIAWNSNKLDSKPSGNYNYRYRLFRKSKLKPFERDISTEIYQAGN |
| | | KPCNGVAGSNCYSPLQSYGFRPTYGVGHQPYRVVVLSFELLHAPATVC |
| | | GPKKSTNLVKNK |
| 710 | XBB | NITNLCPFHEVFNATTFASVYAWNRKRISNCVADYSVLYNFAPFFAFKC |
| | fragment 2 | YGVSPTKLNDLCFTNVYADSFVIRGNEVSQIAPGQTGNIADYNYKLPDD |
| | (AA) | FTGCVIAWNSNKLDSKPSGNYNYRYRLFRKSKLKPFERDISTEIYQAGN |
| | | KPCNGVAGSNCYSPLQSYGFRPTYGVGHQPYRVVVLSFELLHAPATVC |
| | | GPKKSTNLV |
| 711 | XBB | ITNLCPFHEVFNATTFASVYAWNRKRISNCVADYSVLYNFAPFFAFKCY |
| | fragment 3 | GVSPTKLNDLCFTNVYADSFVIRGNEVSQIAPGQTGNIADYNYKLPDDF |
| | (AA) | TGCVIAWNSNKLDSKPSGNYNYRYRLFRKSKLKPFERDISTEIYQAGNK |
| | | PCNGVAGSNCYSPLQSYGFRPTYGVGHQPYRVVVLSFELLHAPATVCG |
| | | PKKSTNLVKNK |
| 712 | XBB | ITNLCPFHEVFNATTFASVYAWNRKRISNCVADYSVLYNFAPFFAFKCY |
| | fragment 4 | GVSPTKLNDLCFTNVYADSFVIRGNEVSQIAPGQTGNIADYNYKLPDDF |
| | (AA) | TGCVIAWNSNKLDSKPSGNYNYRYRLFRKSKLKPFERDISTEIYQAGNK |
| | | PCNGVAGSNCYSPLQSYGFRPTYGVGHQPYRVVVLSFELLHAPATVCG |
| | | PKKSTNLV |
| 713 | BA.4/5 | NITNLCPFDEVFNATRFASVYAWNRKRISNCVADYSVLYNFAPFFAFKC |
| | fragment | YGVSPTKLNDLCFTNVYADSFVIRGNEVSQIAPGQTGNIADYNYKLPDD |
| | (AA) | FTGCVIAWNSNKLDSKVGGNYNYRYRLFRKSNLKPFERDISTEIYQAGN |
| | | KPCNGVAGVNCYFPLQSYGFRPTYGVGHQPYRVVVLSFELLHAPATVC |
| | | GPKKSTNLVKNK |
| 714 | BA.2.75 | NITNLCPFHEVFNATRFASVYAWNRKRISNCVADYSVLYNFAPFFAFKC |
| | fragment | YGVSPTKLNDLCFTNVYADSFVIRGNEVSQIAPGQTGNIADYNYKLPDD |
| | (AA) | FTGCVIAWNSNKLDSKVSGNYNYLYRLFRKSKLKPFERDISTEIYQAGN |
| | | KPCNGVAGFNCYFPLQSYGFRPTYGVGHQPYRVVVLSFELLHAPATVC |
| | | GPKKSTNLVKNK |
| 715 | BA 2.75.2 | NITNLCPFHEVFNATTFASVYAWNRKRISNCVADYSVLYNFAPFFAFKC |
| | fragment | YGVSPTKLNDLCFTNVYADSFVIRGNEVSQIAPGQTGNIADYNYKLPDD |
| | (AA) | FTGCVIAWNSNKLDSKVSGNYNYLYRLFRKSKLKPFERDISTEIYQAGN |
| | | KPCNGVAGSNCYFPLQSYGFRPTYGVGHQPYRVVVLSFELLHAPATVC |
| | | GPKKSTNLVKNK |
| 716 | BA 4.6 | NITNLCPFDEVFNATTFASVYAWNRKRISNCVADYSVLYNFAPFFAFKC |
| | fragment | YGVSPTKLNDLCFTNVYADSFVIRGNEVSQIAPGQTGNIADYNYKLPDD |
| | (AA) | FTGCVIAWNSNKLDSKVGGNYNYRYRLFRKSNLKPFERDISTEIYQAGN |
| | | KPCNGVAGVNCYFPLQSYGFRPTYGVGHQPYRVVVLSFELLHAPATVC |
| | | GPKKSTNLVKNK |
| 717 | BQ.1.1 | AACATAACCAATCTGTGCCCCTTCGACGAGGTATTTAATGCAACTAC |
| | fragment 1 | ATTCGCTAGCGTCTACGCTTGGAATCGGAAACGCATATCTAACTGTG |
| | (Nucl.) | TAGCAGATTATTCCGTACTTTATAACTTCGCTCCCTTTTTCGCTTTCA |
| | | AATGTTACGGAGTGAGCCCTACAAAATTGAACGATCTCTGCTTCACC |
| | | AACGTCTATGCAGACAGTTTTGTTATCCGTGGCAATGAAGTGAGCCA |
| | | GATTGCACCCGGTCAAACTGGGAACATTGCCGACTATAACTACAAA |
| | | CTTCCCGACGACTTCACAGGGTGTGTAATCGCTTGGAACAGCAATAA |
| | | ACTCGACTCTACCGTTGGAGGAAACTACAATTATCGCTACCGCCTTT |
| | | TCCGGAAGTCTAAACTCAAGCCCTTTGAGCGGGACATTAGCACTGA |
| | | GATATATCAGGCAGGTAACAAACCATGCAATGGAGTAGCTGGTGTA |
| | | AACTGCTATTTCCCACTTCAGTCATACGGATTTAGACCCACATACGG |
| | | TGTTGGACACCAACCATATCGTGTGGTTGTGCTCAGTTTTGAACTCC |
| | | TTCATGCCCCTGCCACCGTGTGTGGGCCTAAAAAGTCCACCAACTTG |
| | | GTCAAGAATAAA |
| 718 | BQ.1.1 | AACATAACCAATCTGTGCCCCTTCGACGAGGTATTTAATGCAACTAC |
| | fragment 2 | ATTCGCTAGCGTCTACGCTTGGAATCGGAAACGCATATCTAACTGTG |
| | (Nucl.) | TAGCAGATTATTCCGTACTTTATAACTTCGCTCCCTTTTTCGCTTTCA |
| | | AATGTTACGGAGTGAGCCCTACAAAATTGAACGATCTCTGCTTCACC |
| | | AACGTCTATGCAGACAGTTTTGTTATCCGTGGCAATGAAGTGAGCCA |
| | | GATTGCACCCGGTCAAACTGGGAACATTGCCGACTATAACTACAAA |
| | | CTTCCCGACGACTTCACAGGGTGTGTAATCGCTTGGAACAGCAATAA |
| | | ACTCGACTCTACCGTTGGAGGAAACTACAATTATCGCTACCGCCTTT |
| | | TCCGGAAGTCTAAACTCAAGCCCTTTGAGCGGGACATTAGCACTGA |
| | | GATATATCAGGCAGGTAACAAACCATGCAATGGAGTAGCTGGTGTA |
| | | AACTGCTATTTCCCACTTCAGTCATACGGATTTAGACCCACATACGG |
| | | TGTTGGACACCAACCATATCGTGTGGTTGTGCTCAGTTTTGAACTCC |
| | | TTCATGCCCCTGCCACCGTGTGTGGGCCTAAAAAGTCCACCAACTTG |
| | | GTC |
| 719 | BQ.1.1 | ATAACCAATCTGTGCCCCTTCGACGAGGTATTTAATGCAACTACATT |
| | fragment 3 | CGCTAGCGTCTACGCTTGGAATCGGAAACGCATATCTAACTGTGTAG |
| | (Nucl.) | CAGATTATTCCGTACTTTATAACTTCGCTCCCTTTTTCGCTTTCAAAT |
| | | GTTACGGAGTGAGCCCTACAAAATTGAACGATCTCTGCTTCACCAAC |
| | | GTCTATGCAGACAGTTTTGTTATCCGTGGCAATGAAGTGAGCCAGAT |
| | | TGCACCCGGTCAAACTGGGAACATTGCCGACTATAACTACAAACTTC |
| | | CCGACGACTTCACAGGGTGTGTAATCGCTTGGAACAGCAATAAACT |
| | | CGACTCTACCGTTGGAGGAAACTACAATTATCGCTACCGCCTTTTCC |
| | | GGAAGTCTAAACTCAAGCCCTTTGAGCGGGACATTAGCACTGAGAT |
| | | ATATCAGGCAGGTAACAAACCATGCAATGGAGTAGCTGGTGTAAAC |
| | | TGCTATTTCCCACTTCAGTCATACGGATTTAGACCCACATACGGTGT |
| | | TGGACACCAACCATATCGTGTGGTTGTGCTCAGTTTTGAACTCCTTC |
| | | ATGCCCCTGCCACCGTGTGTGGGCCTAAAAAGTCCACCAACTTGGTC |
| | | AAGAATAAA |
| 720 | BQ.1.1 | ATAACCAATCTGTGCCCCTTCGACGAGGTATTTAATGCAACTACATT |
| | fragment 4 | CGCTAGCGTCTACGCTTGGAATCGGAAACGCATATCTAACTGTGTAG |
| | (Nucl.) | CAGATTATTCCGTACTTTATAACTTCGCTCCCTTTTTCGCTTTCAAAT |
| | | GTTACGGAGTGAGCCCTACAAAATTGAACGATCTCTGCTTCACCAAC |
| | | GTCTATGCAGACAGTTTTGTTATCCGTGGCAATGAAGTGAGCCAGAT |
| | | TGCACCCGGTCAAACTGGGAACATTGCCGACTATAACTACAAACTTC |
| | | CCGACGACTTCACAGGGTGTGTAATCGCTTGGAACAGCAATAAACT |
| | | CGACTCTACCGTTGGAGGAAACTACAATTATCGCTACCGCCTTTTCC |
| | | GGAAGTCTAAACTCAAGCCCTTTGAGCGGGACATTAGCACTGAGAT |
| | | ATATCAGGCAGGTAACAAACCATGCAATGGAGTAGCTGGTGTAAAC |
| | | TGCTATTTCCCACTTCAGTCATACGGATTTAGACCCACATACGGTGT |
| | | TGGACACCAACCATATCGTGTGGTTGTGCTCAGTTTTGAACTCCTTC |
| | | ATGCCCCTGCCACCGTGTGTGGGCCTAAAAAGTCCACCAACTTGGTC |
| 721 | XBB | AACATTACTAATTTGTGCCCCTTCCACGAAGTCTTTAATGCCACCAC |
| | fragment 1 | TTTTGCATCCGTCTACGCATGGAACCGTAAGCGTATAAGTAACTGCG |
| | (Nucl.) | TAGCCGATTATTCTGTTTTGTATAACTTCGCCCCCTTCTTCGCATTCA |
| | | AGTGTTATGGCGTCAGCCCAACCAAGCTTAATGACCTCTGTTTTACA |
| | | AACGTCTACGCTGATAGCTTCGTGATACGTGGTAATGAAGTAAGTCA |
| | | AATAGCCCCTGGCCAGACTGGTAACATTGCAGACTACAATTATAAA |
| | | CTTCCAGATGACTTTACTGGGTGTGTAATCGCTTGGAATAGTAATAA |
| | | GCTCGACTCTAAGCCCTCCGGGAATTACAACTACCGATATCGGCTTT |
| | | TCAGAAAGTCTAAGCTCAAACCATTTGAGCGCGACATAAGCACAGA |
| | | GATATATCAAGCAGGCAATAAGCCTTGTAACGGCGTAGCTGGTAGT |
| | | AATTGTTACTCCCCCCTGCAAAGTTATGGGTTCAGGCCAACTTATGG |
| | | GGTGGGGCACCAGCCCTACAGAGTTGTCGTACTGAGCTTTGAGCTGC |
| | | TCCATGCTCCAGCTACAGTGTGTGGACCCAAGAAATCCACTAACTTG |
| | | GTTAAAAATAAG |
| 722 | XBB | AACATTACTAATTTGTGCCCCTTCCACGAAGTCTTTAATGCCACCAC |
| | fragment 2 | TTTTGCATCCGTCTACGCATGGAACCGTAAGCGTATAAGTAACTGCG |
| | (Nucl.) | TAGCCGATTATTCTGTTTTGTATAACTTCGCCCCCTTCTTCGCATTCA |
| | | AGTGTTATGGCGTCAGCCCAACCAAGCTTAATGACCTCTGTTTTACA |
| | | AACGTCTACGCTGATAGCTTCGTGATACGTGGTAATGAAGTAAGTCA |
| | | AATAGCCCCTGGCCAGACTGGTAACATTGCAGACTACAATTATAAA |
| | | CTTCCAGATGACTTTACTGGGTGTGTAATCGCTTGGAATAGTAATAA |
| | | GCTCGACTCTAAGCCCTCCGGGAATTACAACTACCGATATCGGCTTT |
| | | TCAGAAAGTCTAAGCTCAAACCATTTGAGCGCGACATAAGCACAGA |
| | | GATATATCAAGCAGGCAATAAGCCTTGTAACGGCGTAGCTGGTAGT |
| | | AATTGTTACTCCCCCCTGCAAAGTTATGGGTTCAGGCCAACTTATGG |
| | | GGTGGGGCACCAGCCCTACAGAGTTGTCGTACTGAGCTTTGAGCTGC |
| | | TCCATGCTCCAGCTACAGTGTGTGGACCCAAGAAATCCACTAACTTG |
| | | GTT |
| 723 | XBB | ATTACTAATTTGTGCCCCTTCCACGAAGTCTTTAATGCCACCACTTTT |
| | fragment 3 | GCATCCGTCTACGCATGGAACCGTAAGCGTATAAGTAACTGCGTAG |
| | (Nucl.) | CCGATTATTCTGTTTTGTATAACTTCGCCCCCTTCTTCGCATTCAAGT |
| | | GTTATGGCGTCAGCCCAACCAAGCTTAATGACCTCTGTTTTACAAAC |
| | | GTCTACGCTGATAGCTTCGTGATACGTGGTAATGAAGTAAGTCAAAT |
| | | AGCCCCTGGCCAGACTGGTAACATTGCAGACTACAATTATAAACTTC |
| | | CAGATGACTTTACTGGGTGTGTAATCGCTTGGAATAGTAATAAGCTC |
| | | GACTCTAAGCCCTCCGGGAATTACAACTACCGATATCGGCTTTTCAG |
| | | AAAGTCTAAGCTCAAACCATTTGAGCGCGACATAAGCACAGAGATA |
| | | TATCAAGCAGGCAATAAGCCTTGTAACGGCGTAGCTGGTAGTAATT |
| | | GTTACTCCCCCCTGCAAAGTTATGGGTTCAGGCCAACTTATGGGGTG |
| | | GGGCACCAGCCCTACAGAGTTGTCGTACTGAGCTTTGAGCTGCTCCA |
| | | TGCTCCAGCTACAGTGTGTGGACCCAAGAAATCCACTAACTTGGTTA |
| | | AAAATAAG |
| 724 | XBB | ATTACTAATTTGTGCCCCTTCCACGAAGTCTTTAATGCCACCACTTTT |
| | fragment 4 | GCATCCGTCTACGCATGGAACCGTAAGCGTATAAGTAACTGCGTAG |
| | (Nucl.) | CCGATTATTCTGTTTTGTATAACTTCGCCCCCTTCTTCGCATTCAAGT |
| | | GTTATGGCGTCAGCCCAACCAAGCTTAATGACCTCTGTTTTACAAAC |
| | | GTCTACGCTGATAGCTTCGTGATACGTGGTAATGAAGTAAGTCAAAT |
| | | AGCCCCTGGCCAGACTGGTAACATTGCAGACTACAATTATAAACTTC |
| | | CAGATGACTTTACTGGGTGTGTAATCGCTTGGAATAGTAATAAGCTC |
| | | GACTCTAAGCCCTCCGGGAATTACAACTACCGATATCGGCTTTTCAG |
| | | AAAGTCTAAGCTCAAACCATTTGAGCGCGACATAAGCACAGAGATA |
| | | TATCAAGCAGGCAATAAGCCTTGTAACGGCGTAGCTGGTAGTAATT |
| | | GTTACTCCCCCCTGCAAAGTTATGGGTTCAGGCCAACTTATGGGGTG |
| | | GGGCACCAGCCCTACAGAGTTGTCGTACTGAGCTTTGAGCTGCTCCA |
| | | TGCTCCAGCTACAGTGTGTGGACCCAAGAAATCCACTAACTTGGTT |
| 725 | BA.4/5 | AACATCACTAATTTGTGTCCCTTTGACGAGGTATTCAACGCCACTCG |
| | fragment | CTTTGCTTCTGTCTATGCCTGGAACCGAAAAAGAATTTCTAATTGCG |
| | (Nucl.) | TCGCTGACTACTCCGTGCTCTACAACTTCGCACCTTTCTTCGCTTTCA |
| | | AGTGTTACGGTGTTTCCCCCACTAAACTTAATGATCTGTGCTTTACTA |
| | | ACGTGTACGCAGACTCATTCGTTATTCGGGGTAACGAGGTTTCCCAA |
| | | ATCGCTCCCGGTCAAACCGGAAACATCGCTGATTACAACTATAAGTT |
| | | GCCTGATGACTTTACTGGGTGTGTCATCGCATGGAATTCCAACAAAC |
| | | TGGATAGTAAAGTTGGAGGTAACTACAATTACCGATACAGGCTTTTC |
| | | CGTAAATCCAATCTGAAGCCCTTCGAGCGCGATATTAGTACTGAAAT |
| | | ATATCAGGCTGGTAATAAACCTTGCAATGGGGTGGCTGGAGTAAAC |
| | | TGCTACTTTCCTCTTCAGAGTTACGGGTTCAGGCCCACCTATGGAGT |
| | | CGGACATCAACCATACCGTGTGGTTGTACTGTCATTTGAACTCCTTC |
| | | ACGCTCCCGCTACCGTGTGCGGCCCAAAGAAAAGTACAAACTTGGT |
| | | TAAAAACAAG |
| 726 | BA.2.75 | AATATAACTAACCTTTGCCCATTTCATGAAGTTTTCAATGCCACTAG |
| | fragment | ATTTGCATCCGTATATGCATGGAACCGTAAACGAATTAGTAATTGCG |
| | (Nucl.) | TCGCAGACTATAGTGTTCTTTACAATTTCGCTCCCTTTTTCGCCTTCA |
| | | AGTGTTATGGGGTCTCTCCTACTAAACTCAATGACCTGTGCTTTACC |
| | | AATGTGTACGCCGATTCCTTCGTTATACGAGGGAATGAGGTAAGTCA |
| | | AATAGCCCCTGGACAAACCGGCAACATTGCTGATTACAATTACAAA |
| | | CTGCCTGACGATTTCACCGGTTGTGTAATAGCCTGGAATTCCAATAA |
| | | ACTGGATTCTAAAGTATCAGGCAACTACAACTATCTTTACAGGTTGT |
| | | TTAGAAAAAGCAAGCTGAAGCCCTTCGAGCGCGATATTTCAACAGA |
| | | GATATATCAAGCAGGAAATAAACCATGCAACGGCGTCGCCGGCTTC |
| | | AATTGCTACTTCCCTTTGCAAAGTTATGGTTTTCGACCTACATACGGT |
| | | GTCGGTCATCAGCCCTACCGCGTGGTCGTCCTGTCCTTTGAACTTCTT |
| | | CACGCCCCAGCCACCGTATGTGGTCCCAAGAAGTCAACTAATCTCGT |
| | | CAAAAATAAA |
| 727 | BA 2.75.2 | AATATCACTAATTTGTGTCCCTTCCATGAGGTTTTCAATGCTACAAC |
| | fragment | ATTCGCCAGCGTCTACGCCTGGAACCGTAAGCGAATTTCAAACTGCG |
| | (Nucl.) | TTGCAGACTATAGCGTGCTCTACAATTTTGCACCCTTTTTCGCCTTCA |
| | | AGTGTTACGGTGTGAGCCCTACTAAGTTGAATGACCTCTGTTTTACA |
| | | AATGTGTACGCTGACTCATTCGTGATACGTGGTAACGAAGTGAGTCA |
| | | AATAGCCCCTGGGCAAACAGGTAACATCGCTGACTATAACTACAAG |
| | | TTGCCCGACGATTTTACAGGGTGTGTTATAGCATGGAACAGTAACAA |
| | | GCTCGATTCAAAGGTGAGCGGAAATTACAACTACCTCTATCGACTCT |
| | | TTAGGAAATCCAAGCTCAAGCCATTCGAGAGAGATATATCAACCGA |
| | | AATTTACCAGGCTGGTAACAAGCCATGCAATGGGGTCGCAGGATCC |
| | | AACTGTTATTTCCCATTGCAGTCATACGGCTTCAGGCCCACTTATGG |
| | | GGTAGGTCATCAACCCTACCGGGTCGTCGTCCTTTCTTTTGAACTTCT |
| | | GCACGCACCAGCCACCGTATGCGGCCCCAAGAAGAGTACTAACCTG |
| | | GTGAAGAACAAA |
| 728 | BA.4.6 | AATATTACCAACCTGTGCCCCTTCGATGAGGTTTTCAACGCCACAAC |
| | fragment | TTTTGCAAGTGTTTACGCCTGGAACCGAAAGCGTATAAGCAATTGTG |
| | (Nucl.) | TTGCAGATTATAGTGTGCTCTATAACTTCGCTCCCTTCTTTGCTTTTA |
| | | AATGCTACGGAGTCTCCCCTACAAAGCTGAACGACCTTTGCTTCACT |
| | | AATGTCTACGCCGATAGCTTTGTTATCAGAGGAAATGAAGTATCACA |
| | | GATCGCACCAGGTCAGACAGGCAACATCGCAGACTATAACTATAAA |
| | | CTTCCCGACGATTTTACTGGCTGTGTCATTGCTTGGAACTCAAATAA |
| | | ACTCGACTCAAAGGTGGGAGGAAATTACAACTATCGTTATAGGCTG |
| | | TTCAGGAAAAGTAATCTCAAGCCATTCGAGCGAGACATTAGTACAG |
| | | AGATTTACCAGGCTGGAAACAAACCTTGCAATGGTGTCGCAGGCGT |
| | | CAACTGCTACTTCCCATTGCAGAGCTACGGCTTCAGGCCAACATACG |
| | | GTGTGGGACACCAGCCTTACCGAGTTGTGGTCCTGTCCTTTGAACTT |
| | | CTCCACGCACCTGCTACCGTCTGTGGACCCAAAAAAAGCACCAATCT |
| | | TGTAAAGAATAAA |

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A recombinant polypeptide comprising at least one immunogenic fragment of Severe Acute Respiratory Syndrome Coronavirus 2 (SARS-COV-2) spike glycoprotein and an antibody Fc region.

2. The recombinant polypeptide of claim 1, wherein the at least one fragment of the SARS-COV-2 spike glycoprotein comprises the N-terminal domain of the S1 subunit, the C-terminal domain of the S1 subunit, or both.

3. The recombinant polypeptide of claim 1, wherein the at least one fragment of the SARS-COV-2 spike glycoprotein comprises an amino acid sequence with at least 93% sequence identity to an amino acid sequence selected from the group consisting of SEQ ID NO: 245 (CTD_short_h)₂), SEQ ID NO: 249 (LS2435 Single domain_(GAMMA-Plus)), SEQ ID NO: 253 (BETA_CTD_short_i), SEQ ID NO: 257 (GAMMA_CTD_short_i), SEQ ID NO: 261 (GAMMA_CTD_short_i), SEQ ID NO: 265 (DELTA-Plus_CTD_short_i), SEQ ID NO: 269 (MU_CTD_short_i), SEQ ID NO: 273 (A.30_CTD_short_i), SEQ ID NO: 277 (DELTA-Plus+MU_CTD_short_i), SEQ ID NO: 281 (DELTA-Plus+A.30_CTD_short_i), SEQ ID NO: 285 (BETA_CTD_short_i)₂), SEQ ID NO: 289 ((GAMMA_CTD_short_i)₂), SEQ ID NO: 293 ((DELTA_CTD_short_i)₂), SEQ ID NO: 297 ((DELTA-Plus_CTD_short_i)₂), SEQ ID NO: 301 ((MU_CTD_short_i)₂), SEQ ID NO: 305 ((A.30_CTD_short_i)₂), SEQ ID NO: 309 ((DELTA-Plus+MU_CTD_short_i)₂), SEQ ID NO: 313 ((DELTA-Plus+A.30_CTD_short_i)₂), SEQ ID NO: 317 (Omicron_Boost_CTD_short_i), SEQ ID NO: 321 ((Omicron_Boost_CTD_short_i)₂), SEQ ID NO: 325 (Omicron_CTD_short_i), SEQ ID NO: 329 ((Omicron_CTD_short_i)₂), SEQ ID NO: 333 (OΔ+μ_1_CTD_short_i), SEQ ID NO: 337 ((θΔ+μ_1_CTD_short_i)₂), SEQ ID NO: 341 (OΔ+μ_2_CTD_short_i), SEQ ID NO: 345 ((OΔ+ρ_2_CTD_short_i)₂), SEQ ID NO: 349 (OΔ+μ_3_CTD_short_i), SEQ ID NO: 353 ((OΔ+μ_3_CTD_short_i)₂), SEQ ID NO: 357 (OΔ+μ_4_CTD_short_i), and SEQ ID NO: 361 ((OΔ+μ_4_CTD_short_i)₂), SEQ ID NO: 365 (CTD-gCd01), SEQ ID NO: 366 (CTD-gCd02), SEQ ID NO: 367 (CTD-gCd03), SEQ ID NO: 368 (CTD-gCd04), SEQ ID NO: 369 (CTD-gCd05), SEQ ID NO: 370 (CTD-gCd06), SEQ ID NO: 371 (CTD-gCd07), SEQ ID NO: 372 (CTD-gCd08), SEQ ID NO: 373 (CTD-gCd09), SEQ ID NO: 374 (CTD-gCd10), SEQ ID NO: 375 (CTD-gCd11), SEQ ID NO: 376 (CTD-gCd12), SEQ ID NO: 377 (CTD-gCd13), SEQ ID NO: 378 (CTD-gCd14), SEQ ID NO: 379 (CTD-gCd15), SEQ ID NO: 380 (CTD-gCd16), SEQ ID NO: 381 (CTD-gCd17), SEQ ID NO: 382 (CTD-gCd18), SEQ ID NO: 383 (CTD-gCd19), SEQ ID NO: 384 (CTD-gCd20), SEQ ID NO: 385 (CTD-gCd21), SEQ ID NO: 386 (CTD-gCd22), SEQ ID NO: 387 (CTD-gCd23), SEQ ID NO: 388 (CTD-gCd24), SEQ ID NO: 389 (CTD-gCd25), SEQ ID NO: 390 (CTD-gCd26), SEQ ID NO: 391 (CTD-gCd27), SEQ ID NO: 392 (CTD-gCd28), SEQ ID NO: 393 (CTD-gCd29), SEQ ID NO: 394 (CTD-gCd30), SEQ ID NO: 395 (CTD-gCd31), SEQ ID NO: 396 (CTD-gCd32), SEQ ID NO: 397 (CTD-gCd33), SEQ ID NO: 398 (CTD-gCd34), SEQ ID NO: 399 (CTD-gCd35), SEQ ID NO: 400 (CTD-gCd36), SEQ ID NO: 401 (CTD-gCd37), SEQ ID NO: 402 (CTD-gCd38), SEQ ID NO: 403 (CTD-gN01), SEQ ID NO: 404 (CTD-gN02), SEQ ID NO: 405 (CTD-gN03), SEQ ID NO: 406 (CTD-gN04), SEQ ID NO: 407 (CTD-gN05), SEQ ID NO: 408 (CTD-gN06), SEQ ID NO: 409 (CTD-gN07), SEQ ID NO: 410 (CTD-gN08), SEQ ID NO: 411 (CTD-gN09), SEQ ID NO: 412 (CTD-gN10), SEQ ID NO: 413 (CTD-gN11), SEQ ID NO: 414 (CTD-gN12), SEQ ID NO: 415 (CTD-gN13) SEQ ID NO: 416 (CTD-gN14), SEQ ID NO: 417 (CTD-gN15), SEQ ID NO: 418 (CTD-gN16), SEQ ID NO: 419 (CTD-gN17), SEQ ID NO: 420 (CTD-gN18), SEQ ID NO: 421 (CTD-gN19), SEQ ID NO: 422 (CTD-gN20), SEQ ID NO: 423 (CTD-gN21), SEQ ID NO: 424 (CTD-gN22), SEQ ID NO: 425 (CTD-gN23), SEQ ID NO: 426 (CTD-gN24), SEQ ID NO: 427 (CTD-gN25), SEQ ID NO: 428 (CTD-gN26), SEQ ID NO: 429 (CTD-gN27), SEQ ID NO: 430 (CTD-gN28), SEQ ID NO: 431 (CTD-gN29), SEQ ID NO: 432 (CTD-gN30), SEQ ID NO: 433 (CTD-gN31), SEQ ID NO: 434 (CTD-gN32), SEQ ID NO: 435 (CTD-gN33), SEQ ID NO: 436 (CTD-gN34), SEQ ID NO: 437 (CTD-gN35), SEQ ID NO: 438 (CTD-gN36), SEQ ID NO: 439 (CTD-gN37), SEQ ID NO: 440 (CTD-gN38), SEQ ID NO: 441 (CTD-gN39), SEQ ID NO: 442 (CTD-gN40), SEQ ID NO: 443 (CTD-gN41), SEQ ID NO: 444 (CTD-gN42), SEQ ID NO: 445 (CTD-gN43), SEQ ID NO: 446 (CTD-gN44), SEQ ID NO: 447 (CTD-gN45), SEQ ID NO: 448 (CTD-gN46), SEQ ID NO: 449 (CTD-gN47), and SEQ ID NOs: 705-716.

4. The recombinant polypeptide of claim 1, wherein the polypeptide comprises an amino acid sequence selected from the group consisting of SEQ ID NOS: 245, 249, 253, 257, 261, 265, 269, 273, 277, 281, 285, 289, 293, 297, 301, 305, 309, 313, 317, 321, 325, 329, 333, 337, 341, 345, 349, 353, 357, 361 365-449, and 705-716.

5. The recombinant polypeptide of claim 1, wherein the polypeptide comprises at least two immunogenic fragments.

6. The recombinant polypeptide of claim 5, wherein each of the at least two immunogenic fragments comprises an amino acid sequence with at least 93%, sequence identity to an amino acid sequence selected from the group consisting of SEQ ID NO: 245 (CTD_short_h) 2), SEQ ID NO: 249 (LS2435 Single domain_(GAMMA-Plus)), SEQ ID NO: 253 (BETA_CTD_short_i), SEQ ID NO: 257 (GAMMA_CTD_short_i), SEQ ID NO: 261 (GAMMA_CTD_short_i), SEQ ID NO: 265 (DELTA-Plus_CTD_short_i), SEQ ID NO: 269 (MU_CTD_short_i), SEQ ID NO: 273 (A,30_CTD_short_i), SEQ ID NO: 277 (DELTA-Plus+MU_CTD_short_i), SEQ ID NO: 281 (DELTA-Plus+A.30_CTD_short_i), SEQ ID NO: 285 (BETA_CTD_short_i)₂), SEQ ID NO: 289 ((GAMMA_CTD_short_i)₂), SEQ ID NO: 293 ((DELTA_CTD_short_i)₂), SEQ ID NO: 297 ((DELTA-Plus_CTD_short_i)₂), SEQ ID NO: 301 ((MU_CTD_short_i)₂), SEQ ID NO: 305 ((A.30_CTD_short_i)₂), SEQ ID NO: 309 ((DELTA-Plus+MU_CTD_short_i)₂), SEQ ID NO: 313 ((DELTA-Plus+A.30_CTD_short_i)₂), SEQ ID NO: 317 (Omicron_Boost_CTD_short_i), SEQ ID NO: 321 ((Omicron_Boost_CTD_short_i)₂), SEQ ID NO: 325 (Omicron_CTD_short_i), SEQ ID NO: 329 ((Omicron_CTD_short_i)₂), SEQ ID NO: 333 (OΔ+μ_1_CTD_short_i), SEQ ID NO: 337 ((OΔ+μ_1_CTD_short_i)₂), SEQ ID NO: 341 (OΔ+μ_2_CTD_short_i), SEQ ID NO: 345 ((OΔ+μ_2_CTD_short_i)₂), SEQ ID NO: 349 (OΔ+μ_3_CTD_short_i), SEQ ID NO: 353 ((OΔ+μ_3_CTD_short_i)₂), SEQ ID NO: 357 (OΔ+μ_4_CTD_short_i), SEQ ID NO: 361 ((OΔ+μ_4_CTD_short_i)₂), SEQ ID NO: 365 (CTD-gCd01), SEQ ID NO: 366 (CTD-gCd02), SEQ ID NO: 367 (CTD-gCd03), SEQ ID NO: 368 (CTD-gCd04), SEQ ID NO: 369 (CTD-gCd05), SEQ ID NO: 370 (CTD-gCd06), SEQ ID NO: 371 (CTD-gCd07), SEQ ID NO: 372 (CTD-gCd08), SEQ ID NO: 373 (CTD-gCd09), SEQ ID NO: 374 (CTD-gCd10), SEQ ID NO: 375 (CTD-gCd11), SEQ ID NO: 376 (CTD-gCd12), SEQ ID NO: 377 (CTD-gCd13), SEQ ID NO: 378 (CTD-gCd14), SEQ ID NO: 379 (CTD-gCd15), SEQ ID NO: 380 (CTD-gCd16), SEQ ID NO: 381 (CTD-gCd17), SEQ ID NO: 382 (CTD-gCd18), SEQ ID NO: 383 (CTD-gCd19), SEQ ID NO: 384 (CTD-gCd20), SEQ ID NO: 385 (CTD-gCd21), SEQ ID NO: 386 (CTD-gCd22), SEQ ID NO: 387 (CTD-gCd23), SEQ ID NO: 388 (CTD-gCd24), SEQ ID NO: 389 (CTD-gCd25), SEQ ID NO: 390 (CTD-gCd26), SEQ ID NO: 391 (CTD-gCd27), SEQ ID NO: 392 (CTD-gCd28), SEQ ID NO: 393 (CTD-gCd29), SEQ ID NO: 394 (CTD-gCd30), SEQ ID NO: 395 (CTD-gCd31), SEQ ID NO: 396 (CTD-gCd32), SEQ ID NO: 397 (CTD-gCd33), SEQ ID NO: 398 (CTD-gCd34), SEQ ID NO: 399 (CTD-gCd35), SEQ ID NO: 400 (CTD-gCd36), SEQ ID NO: 401 (CTD-gCd37), SEQ ID NO: 402 (CTD-gCd38), SEQ ID NO: 403 (CTD-gN01), SEQ ID NO: 404 (CTD-gN02), SEQ ID NO: 405 (CTD-gN03), SEQ ID NO: 406 (CTD-gN04), SEQ ID NO: 407 (CTD-gN05), SEQ ID NO: 408 (CTD-gN06), SEQ ID NO: 409 (CTD-gN07), SEQ ID NO: 410 (CTD-gN08), SEQ ID NO: 411 (CTD-gN09), SEQ ID NO: 412 (CTD-gN10), SEQ ID NO: 413 (CTD-gN11), SEQ ID NO: 414 (CTD-gN12), SEQ ID NO: 415 (CTD-gN13) SEQ ID NO: 416 (CTD-gN14), SEQ ID NO: 417 (CTD-gN15), SEQ ID NO: 418 (CTD-gN16), SEQ ID NO: 419 (CTD-gN17), SEQ ID NO: 420 (CTD-gN18), SEQ ID NO: 421 (CTD-gN19), SEQ ID NO: 422 (CTD-gN20), SEQ ID NO: 423 (CTD-gN21), SEQ ID NO: 424 (CTD-gN22), SEQ ID NO: 425 (CTD-gN23), SEQ ID NO: 426 (CTD-gN24), SEQ ID NO: 427 (CTD-gN25), SEQ ID NO: 428 (CTD-gN26), SEQ ID NO: 429 (CTD-gN27), SEQ ID NO: 430 (CTD-gN28), SEQ ID NO: 431 (CTD-gN29), SEQ ID NO: 432 (CTD-gN30), SEQ ID NO: 433 (CTD-gN31), SEQ ID NO: 434 (CTD-gN32), SEQ ID NO: 435 (CTD-gN33), SEQ ID NO: 436 (CTD-gN34), SEQ ID NO: 437 (CTD-gN35), SEQ ID NO: 438 (CTD-gN36), SEQ ID NO: 439 (CTD-gN37), SEQ ID NO: 440 (CTD-gN38), SEQ ID NO: 441 (CTD-gN39), SEQ ID NO: 442 (CTD-gN40), SEQ ID NO: 443 (CTD-gN41), SEQ ID NO: 444 (CTD-gN42), SEQ ID NO: 445 (CTD-gN43), SEQ ID NO: 446 (CTD-gN44), SEQ ID NO: 447 (CTD-gN45), SEQ ID NO: 448 (CTD-gN46), SEQ ID NO: 449 (CTD-gN47), and SEQ ID NOs: 705-716.

7. The recombinant polypeptide of claim 5, wherein each of the at least two immunogenic fragments comprises an amino acid sequence selected from the group consisting of SEQ ID NOS: 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365-449, and 705-716.

8. The recombinant polypeptide of claim 5, wherein each immunogenic fragment of the at least two immunogenic fragments comprises the same amino acid sequence.

9. The recombinant polypeptide of claim 5, wherein each immunogenic fragment of the at least two immunogenic fragments comprises a different amino acid sequence from the other immunogenic fragments.

10. The recombinant polypeptide of claim 1, wherein the polypeptide comprises two, three, four, or five immunogenic fragments.

11. The recombinant polypeptide of claim 5, wherein the at least two immunogenic fragments are connected to each other via a linker.

12. The recombinant polypeptide of claim 11, wherein the linker is a polypeptide comprising an amino acid sequence of 1-35 residues, wherein each residue is independently serine or glycine.

13. The recombinant polypeptide of claim 1, wherein the at least one immunogenic fragment of the SARS-COV-2 spike glycoprotein is connected to the antibody Fc region via a linker.

14. The recombinant polypeptide of claim 13, wherein the linker comprises an amino acid sequence selected from the group consisting of SEQ ID NO: 65 (Fc1), SEQ ID NO: 67 (Fc1-TEV), SEQ ID NO: 69 (Fc1-Rv3C), SEQ ID NO: 193 (Short), SEQ ID NO: 195 (Medium), and SEQ ID NO: 197 (Long).

15. The recombinant polypeptide of claim 1, wherein the antibody Fc region is from a human IgG1 antibody or derived therefrom.

16. The recombinant polypeptide of claim 15, wherein the antibody Fc region comprises the amino acid sequence of SEQ ID NO: 71.

17. The recombinant polypeptide of claim 1, wherein the polypeptide comprises an amino acid sequence with at least 93%, sequence identity to an amino acid sequence selected from the group consisting of SEQ ID NO: 247 (CTD_short_h) 2-Fc), SEQ ID NO: 251 (LS2435 Single domain_(GAMMA-Plus)-Fc), SEQ ID NO: 255 (BETA_CTD_short_i-Fc), SEQ ID NO: 259 (GAMMA_CTD_short_i-Fc), SEQ ID NO: 263 (GAMMA_CTD_short_i-Fc), SEQ ID NO: 267 (DELTA-Plus_CTD_short_i-Fc), SEQ ID NO: 271 (MU_CTD_short_i-Fc), SEQ ID NO: 275 (A.30_CTD_short_i-Fc), SEQ ID NO: 279 (DELTA-Plus+MU_CTD_short_i-Fc), SEQ ID NO: 283 (DELTA-Plus+A.30_CTD_short i-Fc), SEQ ID NO: 287 (BETA_CTD_short_i)₂-Fc), SEQ ID NO: 291 ((GAMMA_CTD_short_i)₂-Fc), SEQ ID NO: 295 ((DELTA_CTD_short_i)₂-Fc), SEQ ID NO: 299 ((DELTA-Plus_CTD_short_i)₂-Fc), SEQ ID NO: 303 ((MU_CTD_short_i)₂-Fc), SEQ ID NO: 307 ((A.30_CTD_short_i)₂-Fc), SEQ ID NO: 311 ((DELTA-Plus+MU_CTD_short_i)₂-Fc), SEQ ID NO: 315 ((DELTA-Plus+A.30_CTD_short_i)₂-Fc), SEQ ID NO: 319 (Omicron_Boost_CTD_short_i-Fc), SEQ ID NO: 323 ((Omicron_Boost_CTD_short_i)₂-Fc), SEQ ID NO: 327 (Omicron_CTD_short_i-Fc), SEQ ID NO: 331 ((Omicron_CTD_short_i)₂-Fc), SEQ ID NO: 335 (OΔ+μ_1_CTD_short_i-Fc), SEQ ID NO: 339 (OΔ+μ_1_CTD_short_i)₂-Fc), SEQ ID NO: 343 (OA+μ _2_CTD_short_i-Fc), SEQ ID NO: 347 ((OΔ+μ_2_CTD_short_i)₂-Fc), SEQ ID NO: 351 (OΔ+μ_3_CTD_short_i-Fc), SEQ ID NO: 355 ((OΔ+μ_3_CTD_short_i)₂-Fc), SEQ ID NO: 359 (OΔ+μ_4_CTD_short_i-Fc), SEQ ID NO: 363 ((OΔ+μ_4_CTD_short_i)₂-Fc) SEQ ID NO: 535 (CTD-gCd01-Fc), SEQ ID NO: 536 (CTD-gCd02-Fc), SEQ ID NO: 537 (CTD-gCd03-Fc), SEQ ID NO: 538 (CTD-gCd04-Fc), SEQ ID NO: 539 (CTD-gCd05-Fc), SEQ ID NO: 540 (CTD-gCd06-Fc), SEQ ID NO: 541 (CTD-gCd07-Fc), SEQ ID NO: 542 (CTD-gCd08-Fc), SEQ ID NO: 543 (CTD-gCd09-Fc), SEQ ID NO: 544 (CTD-gCd10-Fc), SEQ ID NO: 545 (CTD-gCd11-Fc), SEQ ID NO: 546 (CTD-gCd12-Fc), SEQ ID NO: 547 (CTD-gCd13-Fc), SEQ ID NO: 548 (CTD-gCd14-Fc), SEQ ID NO: 549 (CTD-gCd15-Fc), SEQ ID NO: 550 (CTD-gCd16-Fc), SEQ ID NO: 551 (CTD-gCd17-Fc), SEQ ID NO: 552 (CTD-gCd18-Fc), SEQ ID NO: 553 (CTD-gCd19-Fc), SEQ ID NO: 554 (CTD-gCd20-Fc), SEQ ID NO: 555 (CTD-gCd21-Fc), SEQ ID NO: 556 (CTD-gCd22-Fc), SEQ ID NO: 557 (CTD-gCd23-Fc), SEQ ID NO: 558 (CTD-gCd24-Fc), SEQ ID NO: 559 (CTD-gCd25-Fc), SEQ ID NO: 560 (CTD-gCd26-Fc), SEQ ID NO: 561 (CTD-gCd27-Fc), SEQ ID NO: 562 (CTD-gCd28-Fc), SEQ ID NO: 563 (CTD-gCd29-Fc), SEQ ID NO: 564 (CTD-gCd30-Fc), SEQ ID NO: 565 (CTD-gCd31-Fc), SEQ ID NO: 566 (CTD-gCd32-Fc), SEQ ID NO: 567 (CTD-gCd33-Fc), SEQ ID NO: 568 (CTD-gCd34-Fc), SEQ ID NO: 569 (CTD-gCd35-Fc), SEQ ID NO: 570 (CTD-gCd36-Fc), SEQ ID NO: 571 (CTD-gCd37-Fc), SEQ ID NO: 572 (CTD-gCd38-Fc), SEQ ID NO: 573 (CTD-gN01-Fc), SEQ ID NO: 574 (CTD-gN02), SEQ ID NO: 575 (CTD-gN03-Fc), SEQ ID NO: 576 (CTD-gN04-Fc), SEQ ID NO: 577 (CTD-gN05-Fc), SEQ ID NO: 578 (CTD-gN06-Fc), SEQ ID NO: 579 (CTD-gN07-Fc), SEQ ID NO: 580 (CTD-gN08-Fc), SEQ ID NO: 581 (CTD-gN09-Fc), SEQ ID NO: 582 (CTD-gN10-Fc), SEQ ID NO: 583 (CTD-gN11-Fc), SEQ ID NO: 584 (CTD-gN12-Fc), SEQ ID NO: 585 (CTD-gN13-Fc) SEQ ID NO: 586 (CTD-gN14-Fc), SEQ ID NO: 587 (CTD-gN15-Fc), SEQ ID NO: 588 (CTD-gN16-Fc), SEQ ID NO: 589 (CTD-gN17-Fc), SEQ ID NO: 590 (CTD-gN18-Fc), SEQ ID NO: 591 (CTD-gN19-Fc), SEQ ID NO: 592 (CTD-gN20-Fc), SEQ ID NO: 593 (CTD-gN21-Fc), SEQ ID NO: 594 (CTD-gN22-Fc), SEQ ID NO: 595 (CTD-gN23-Fc), SEQ ID NO: 596 (CTD-gN24-Fc), SEQ ID NO: 597 (CTD-gN25-Fc), SEQ ID NO: 598 (CTD-gN26-Fc), SEQ ID NO: 599 (CTD-gN27-Fc), SEQ ID NO: 600 (CTD-gN28-Fc), SEQ ID NO: 601 (CTD-gN29-Fc), SEQ ID NO: 602 (CTD-gN30-Fc), SEQ ID NO: 603 (CTD-gN31-Fc), SEQ ID NO: 604 (CTD-gN32-Fc), SEQ ID NO: 605 (CTD-gN33-Fc), SEQ ID NO: 606 (CTD-gN34-Fc), SEQ ID NO: 607 (CTD-gN35-Fc), SEQ ID NO: 608 (CTD-gN36-Fc), SEQ ID NO: 609 (CTD-gN37-Fc), SEQ ID NO: 610 (CTD-gN38-Fc), SEQ ID NO: 611 (CTD-gN39-Fc), SEQ ID NO: 612 (CTD-gN40-Fc), SEQ ID NO: 613 (CTD-gN41-Fc), SEQ ID NO: 614 (CTD-gN42-Fc), SEQ ID NO: 615 (CTD-gN43-Fc), SEQ ID NO: 616 (CTD-gN44-Fc), SEQ ID NO: 617 (CTD-gN45-Fc), SEQ ID NO: 618 (CTD-gN46-Fc), SEQ ID NO: 619 (CTD-gN47), and SEQ ID NOs: 705-716.

18. The recombinant polypeptide of claim 17, wherein the polypeptide comprises an amino acid sequence selected from the group consisting of SEQ ID NOS: 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 535-619, and 705-716.

19. A recombinant polynucleotide encoding the recombinant polypeptide of claim 1.

20. The recombinant polynucleotide of claim 19, wherein the polynucleotide comprises a nucleic acid sequence with at least 93%, sequence identity to a nucleic acid sequence selected from the group consisting of SEQ ID NO: 248 (CTD_short_h) 2-Fc), SEQ ID NO: 252 (LS2435 Single domain_(GAMMA-Plus)-Fc), SEQ ID NO: 256 (BETA_CTD_short_i-Fc), SEQ ID NO: 260 (GAMMA_CTD_short_i-Fc), SEQ ID NO: 264 (GAMMA_CTD_short_i-Fc), SEQ ID NO: 268 (DELTA-Plus_CTD_short_i-Fc), SEQ ID NO: 272 (MU_CTD_short_i-Fc), SEQ ID NO: 276 (A.30_CTD_short_i-Fc), SEQ ID NO: 280 (DELTA-Plus+MU_CTD_short_i-Fc), SEQ ID NO: 284 (DELTA-Plus+A.30_CTD_short_i-Fc), SEQ ID NO: 288 (BETA_CTD_short_i)₂-Fc), SEQ ID NO: 292 ((GAMMA_CTD_short_i)₂-Fc), SEQ ID NO: 296 ((DELTA_CTD_short_i)₂-Fc), SEQ ID NO: 300 ((DELTA-Plus_CTD_short_i)₂-Fc), SEQ ID NO: 304 ((MU_CTD_short_i)₂-Fc), SEQ ID NO: 308 ((A.30_CTD_short_i)₂-Fc), SEQ ID NO: 312 ((DELTA-Plus+MU_CTD_short_i)₂-Fc), SEQ ID NO: 316 ((DELTA-Plus+A.30_CTD_short_i)₂-Fc), SEQ ID NO: 320 (Omicron_Boost_CTD_short_i-Fc), SEQ ID NO: 324 ((Omicron_Boost_CTD_short_i)₂-Fc), SEQ ID NO: 328 (Omicron_CTD_short_i-Fc), SEQ ID NO: 332 ((Omicron_CTD_short_i)₂-Fc), SEQ ID NO: 336 (OΔ+μ_1_CTD_short_i-Fc), SEQ ID NO: 340 ((OΔ+μ_1_CTD_short_i)₂-Fc), SEQ ID NO: 344 (OΔ+μ_2_CTD_short_i-Fc), SEQ ID NO: 348 ((OA+μ_2_CTD_short_i)₂-Fc), SEQ ID NO: 352 (OΔ+μ_3_CTD_short_i-Fc), SEQ ID NO: 356 ((OΔ+μ_3_CTD_short_i)₂-Fc), SEQ ID NO: 360 (OΔ+μ_4_CTD_short_i-Fc), SEQ ID NO: 364 ((OΔ+μ_4_CTD_short_i)₂-Fc), SEQ ID NO: 620 (CTD-gCd01-Fc), SEQ ID NO: 621 (CTD-gCd02-Fc), SEQ ID NO: 622 (CTD-gCd03-Fc), SEQ ID NO: 623 (CTD-gCd04-Fc), SEQ ID NO: 624 (CTD-gCd05-Fc), SEQ ID NO: 625 (CTD-gCd06-Fc), SEQ ID NO: 626 (CTD-gCd07-Fc), SEQ ID NO: 627 (CTD-gCd08-Fc), SEQ ID NO: 628 (CTD-gCd09-Fc), SEQ ID NO: 629 (CTD-gCd10-Fc), SEQ ID NO: 630 (CTD-gCd11-Fc), SEQ ID NO: 631 (CTD-gCd12-Fc), SEQ ID NO: 632 (CTD-gCd13-Fc), SEQ ID NO: 633 (CTD-gCd14-Fc), SEQ ID NO: 634 (CTD-gCd15-Fc), SEQ ID NO: 635 (CTD-gCd16-Fc), SEQ ID NO: 636 (CTD-gCd17-Fc), SEQ ID NO: 637 (CTD-gCd18-Fc), SEQ ID NO: 638 (CTD-gCd19-Fc), SEQ ID NO: 639 (CTD-gCd20-Fc), SEQ ID NO: 640 (CTD-gCd21-Fc), SEQ ID NO: 641 (CTD-gCd22-Fc), SEQ ID NO: 642 (CTD-gCd23-Fc), SEQ ID NO: 643 (CTD-gCd24-Fc), SEQ ID NO: 644 (CTD-gCd25-Fc), SEQ ID NO: 645 (CTD-gCd26-Fc), SEQ ID NO: 646 (CTD-gCd27-Fc), SEQ ID NO: 647 (CTD-gCd28-Fc), SEQ ID NO: 648 (CTD-gCd29-Fc), SEQ ID NO: 649 (CTD-gCd30-Fc), SEQ ID NO: 650 (CTD-gCd31-Fc), SEQ ID NO: 651 (CTD-gCd32-Fc), SEQ ID NO: 652 (CTD-gCd33-Fc), SEQ ID NO: 653 (CTD-gCd34-Fc), SEQ ID NO: 654 (CTD-gCd35-Fc), SEQ ID NO: 655 (CTD-gCd36-Fc), SEQ ID NO: 656 (CTD-gCd37-Fc), SEQ ID NO: 657 (CTD-gCd38-Fc), SEQ ID NO: 658 (CTD-gN01-Fc), SEQ ID NO: 659 (CTD-gN02), SEQ ID NO: 660 (CTD-gN03-Fc), SEQ ID NO: 661 (CTD-gN04-Fc), SEQ ID NO: 662 (CTD-gN05-Fc), SEQ ID NO: 663 (CTD-gN06-Fc), SEQ ID NO: 664 (CTD-gN07-Fc), SEQ ID NO: 665 (CTD-gN08-Fc), SEQ ID NO: 666 (CTD-gN09-Fc), SEQ ID NO: 667 (CTD-gN10-Fc), SEQ ID NO: 668 (CTD-gN11-Fc), SEQ ID NO: 669 (CTD-gN12-Fc), SEQ ID NO: 670 (CTD-gN13-Fc) SEQ ID NO: 671 (CTD-gN14-Fc), SEQ ID NO: 672 (CTD-gN15-Fc), SEQ ID NO: 673 (CTD-gN16-Fc), SEQ ID NO: 674 (CTD-gN17-Fc), SEQ ID NO: 675 (CTD-gN18-Fc), SEQ ID NO: 676 (CTD-gN19-Fc), SEQ ID NO: 677 (CTD-gN20-Fc), SEQ ID NO: 678 (CTD-gN21-Fc), SEQ ID NO: 679 (CTD-gN22-Fc), SEQ ID NO: 680 (CTD-gN23-Fc), SEQ ID NO: 681 (CTD-gN24-Fc), SEQ ID NO: 682 (CTD-gN25-Fc), SEQ ID NO: 683 (CTD-gN26-Fc), SEQ ID NO: 684 (CTD-gN27-Fc), SEQ ID NO: 685 (CTD-gN28-Fc), SEQ ID NO: 686 (CTD-gN29-Fc), SEQ ID NO: 687 (CTD-gN30-Fc), SEQ ID NO: 688 (CTD-gN31-Fc), SEQ ID NO: 689 (CTD-gN32-Fc), SEQ ID NO: 690 (CTD-gN33-Fc), SEQ ID NO: 691 (CTD-gN34-Fc), SEQ ID NO: 692 (CTD-gN35-Fc), SEQ ID NO: 693 (CTD-gN36-Fc), SEQ ID NO: 694 (CTD-gN37-Fc), SEQ ID NO: 695 (CTD-gN38-Fc), SEQ ID NO: 696 (CTD-gN39-Fc), SEQ ID NO: 697 (CTD-gN40-Fc), SEQ ID NO: 698 (CTD-gN41-Fc), SEQ ID NO: 699 (CTD-gN42-Fc), SEQ ID NO: 700 (CTD-gN43-Fc), SEQ ID NO: 701 (CTD-gN44-Fc), SEQ ID NO: 702 (CTD-gN45-Fc), SEQ ID NO: 703 (CTD-gN46-Fc), SEQ ID NO: 704 (CTD-gN47), and SEQ ID NOs: 717-728.

21. The recombinant polynucleotide of claim 20, wherein the polynucleotide comprises a nucleic acid sequence selected from the group consisting of SEQ ID NO: 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 620-704, and 717-728.

22. The recombinant polynucleotide of claim 19, wherein the nucleic acid sequence has been codon optimized.

23. A pharmaceutical composition comprising the recombinant polypeptide of claim 1, and a pharmaceutically acceptable carrier.

24. The pharmaceutical composition of claim 23, wherein the pharmaceutical composition further comprises at least one adjuvant, and
wherein the at least one adjuvant is selected from the group consisting of alum adjuvants, emulsion adjuvants, and pattern recognition receptor agonist adjuvants, or
wherein the at least one adjuvant is AS03, MF59, CpG, Squalene Emulsion, Alum, aluminum hydroxide gels, calcium phosphate hydroxide, paraffin oil, cytokines (IL-1, IL-2, IL-12), killed bacterial products such as Bordetella and Mycobacterium bacteria, bacterial toxoids, squalene and DL-α-tocopherol emulsions, aluminum phosphate gels, saponins, cyclic dinucleotides, and TLR agonists, and any combination thereof, or
wherein the at least one adjuvant is a squalene-oil-in-water emulsion adjuvant.

25-27. (canceled)

28. A vector comprising the recombinant polynucleotide of claim 19.

29. An isolated cell comprising the polypeptide encoded by the recombinant polynucleotide of claim 19.

30. A method for preventing, inhibiting, reducing, eliminating,
protecting, or delaying the onset of an infection or an infectious clinical condition caused by a beta coronavirus in a subject comprising administering to the subject the recombinant polypeptide of claim 1.

31. A method for inducing an immune response against a beta coronavirus in a subject comprising administering to the subject the recombinant polypeptide of claim 1.

32. The method of claim 30, wherein the recombinant polypeptide is administered by oral, parenteral, subcutaneous, intravenous, intramuscular, intranasal, intrapulmonary, intraarterial, intrathecal, or interperitoneal administration.

33. The method of claim 30, wherein the coronavirus is selected from the group consisting of SARS-COV-2, SARS-COV, and MERS-COV.

34. The method of claim 30, wherein the subject is a mammal.

35-36. (canceled)
